(19) United States
(12) Patent Application Publication
(10) Pub. No.: US 2014/0155420 A1
(43) Pub. Date: Jun. 5, 2014

(54) INHIBITORS OF EYA2

(71) Applicants: Rui Zhao, Denver, CO (US); Heide L. Ford, Denver, CO (US); Noel Southall, Potomac, MD (US); Erika Englund, Washington, DC (US); Samarjit Patnaik, Gaithersburg, MD (US); Marc Ferrer, Potomac, MD (US); Wei Zheng, Potomac, MD (US); Seameen Dehdashti, Poolesville, MD (US); Juan Marugan, Gaithersburg, MD (US)

(72) Inventors: Rui Zhao, Denver, CO (US); Heide L. Ford, Denver, CO (US); Noel Southall, Potomac, MD (US); Erika Englund, Washington, DC (US); Samarjit Patnaik, Gaithersburg, MD (US); Marc Ferrer, Potomac, MD (US); Wei Zheng, Potomac, MD (US); Seameen Dehdashti, Poolesville, MD (US); Juan Marugan, Gaithersburg, MD (US)

(73) Assignees: The Regents of the University of Colorado, a body corporate, Denver, CO (US); Department of Health and Human Services, Rockville, MD (US); The Government of the United States of America as represented by the Secretary of the

(21) Appl. No.: 13/978,820

(22) PCT Filed: Jan. 10, 2012
(86) PCT No.: PCT/US12/20805

§ 371 Date: Feb. 5, 2014

Related U.S. Application Data
(60) Provisional application No. 61/431,306, filed on Jan. 10, 2011.

(51) Int. Cl. A61K 31/443; A61K 31/4174; A61K 31/277; A61K 31/4709; A61K 31/255; A61K 31/155

(52) U.S. Cl. CPC A61K 31/443; A61K 31/255; A61K 31/155; A61K 31/277; A61K 31/4709; A61K 31/4174
USPC 514/274; 514/336; 514/518; 514/637; 514/471; 514/351; 514/369; 514/367; 514/522; 514/313; 514/399

(57) ABSTRACT

The invention provides small molecule inhibitors of EYA2 phosphatase activity and EYA2 binding to Six1. These inhibitors are proposed for use in methods of treating cancer in a subject, such as those involving Six1 and/or EYA2 disregulation. In some embodiments, the invention further provides for the administration of a second cancer therapy to the subject.

### PRIORITY INFORMATION

The present application claims benefit of priority to U.S. Provisional Application Ser. No. 61/431,306, filed Jan. 10, 2011, the entire contents of which are hereby incorporated by reference.

### STATEMENT OF GOVERNMENT SUPPORT

This invention was made with government support under Grant No. CA095277 awarded by the National Institutes of Health, and grant numbers W81XWH-09-0252 and W81XWH-09-1-0253 awarded by the Army Medical Research Material and Command. The government has certain rights in the invention.

### BACKGROUND OF THE INVENTION

1. Field of the Invention

The present invention relates generally to the fields of oncology, molecular biology, and medicine. More particularly, the invention relates to the use of small molecule inhibitors of Eya2 phophatase activity and Six1 binding, including in the context of cancer treatments.

2. Description of Related Art

The Eya proteins are mammalian homologues of the Drosophila Eyes Absent genes and were first identified as essential co-activators of members of the Six family of transcription factors, including Six1. The Six1 homeoprotein is essential for the development of many organs, including the muscle, kidney, olfactory epithelium, and inner ear (Ozaki et al., 2004; Li et al., 2003; Grifone et al., 2005; Ikeda et al., 2007; Zheng et al., 2003; Xu et al., 2003; Laclef et al., 2003a; Laclef et al., 2003b). It is typically down-regulated after organ development is complete, and its expression level is low or absent in most adult tissues (Ford et al., 1998). However, Six1 is over-expressed in numerous cancers, including breast, ovarian, cervical, and hepatocellular carcinomas, as well as rhabdomyosarcomas, Wilms' tumors, and leukemias (Ford et al., 1998; Behbakht et al., 2007; Ng et al., 2006; Wan et al., 2008; Li et al., 2002; Yu et al., 2004; Xu-Dong et al., 2009; Wang et al., 2011). Importantly, Six1 expression has been linked to transformation, tumor growth, and metastasis in multiple tumor types, including breast cancer (Yu et al., 2004; McCoy et al., 2009; Coletta et al., 2004; Yu et al., 2006; Ng et al., 2010; Coletta et al., 2008; Micalizzi et al., 2009). Furthermore, experimentally lowering Six1 levels significantly decreases cancer cell proliferation (Coletta et al., 2004) and metastasis (Yu et al., 2004; Ng et al., 2010) in different cancer models.

Given that Six1 does not have an intrinsic activation or repression domain, it requires co-activators such as the Eya family of proteins to mediate its transcriptional activity, both in normal development (Li et al., 2003; Zhang et al., 2004) and in various disease processes (Zhang et al., 2004; Abdelhak et al., 1997; Ruf et al., 2004). Eya proteins have been linked to many types of cancer in which Six1 is over-expressed (Behbakht et al., 2007; Li et al., 2002; Zhang et al., 2005; Farabaugh et al., 2011; Pandey et al., 2010). Over-expression of Eya2 and Six1 have been independently associated with increased proliferation and shortened overall survival of advanced ovarian cancer patients (Behbakht et al., 2007; Zhang et al., 2005; Zheng et al., 2003). Examination of the Wang and Van de Vijver public breast cancer microarray datasets (Wang et al., 2005; van de Vijver et al., 2002) demonstrated that over-expression of Six1 and Eya2 together significantly predict shortened time to relapse and metastasis (p=0.004, p=0.01 respectively) and shortened survival (p=0.003), whereas each gene individually does not (Farabaugh et al., 2011). Indeed, this correlation holds true when examining cooperativity between Six1 and other Eya family members: Over-expression of Eya1 together with Six1 also significantly correlates with adverse clinical outcomes (Farabaugh et al., 2011). Thus, it is likely that all Eya family members can cooperate with Six1 to confer poor prognosis in breast cancer. Like Six1, most Eya family members are expressed in developing tissues, but not in most normal adult tissues (Abdelhak et al., 1997; Borsani et al., 1999; Zimmerman et al., 1997). To conclusively prove that Eya cooperates with Six1 in inducing pro-tumorigenic and metastatic phenotypes, the inventors knocked down Eya2 in Six1-over-expressing MCF7 cells and demonstrated that the loss of Eya2 in the context of Six1 over-expression inhibits the ability of Six1 to induce TGF-β signaling, epithelial-mesenchymal transition (EMT), and tumor initiating cell (TIC) characteristics, properties that are associated with Six1-induced tumorigenesis and metastasis (Farabaugh et al., 2011). These data provide strong support that Six1 and Eya2 cooperate to induce tumorigenic and metastatic properties.

The Eya proteins have a C-terminal Eya Domain (ED) (Rayapureddi et al., 2003) that contains signature motifs of the haloacid dehalogenase (HAD) hydrolases, a diverse collection of enzymes including phosphatases (Li et al., 2003; Rayapureddi et al., 2003; Tootle et al., 2003). Eya proteins and other HAD family of phosphatases use an Asp as their active site residue instead of the more commonly used Cys in cellular phosphatases (Tonks et al., 2006). A few other HAD phosphatases (for example, Scp1 and Chronophin) target proteins, however, most HAD phosphatases do not have protein phosphatase activity (Rayapureddi et al., 2003). All other known HAD protein phosphatases are Ser/Thr phosphatases (such as Scp1), while the Eya domain of Eya targets phosphorylated Tyr (Krishnan et al., 2009). Recent evidence demonstrates that mouse Eya proteins can utilize their intrinsic phosphatase activity to switch the Six1 transcriptional complex from a repressor to an activator complex for some Six1-induced genes (Li et al., 2003), although the mechanism of this switch remains unclear. In Drosophila, while the phosphatase activity of Eya is not globally required for the ability of Six1 to induce transcription, it is required to induce transcription of a subset of genes (Jemc and Rebay, 2007). The Eya proteins therefore represent the first transcription factor with intrinsic phosphatase activity that modulates transcriptional complexes (Li et al., 2003; Rayapureddi et al., 2003; Tootle et al., 2003). Importantly, using two different breast cancer cell lines and both over-expression and knockdown systems, and examining multiple different Eyas, Hegde and colleagues demonstrated that Eya proteins, and in particular their Tyr phosphatase activity, are critical for transformation, migration, invasion, and metastasis of breast cancer cells (Pandey et al., 2010). Although the specific mechanism by which Eya's phosphatase activity induces the metastatic phenotype is controversial, the fact that only joint over-expression of Six1 and Eya correlates with adverse clinical outcomes (Farabaugh et al., 2011), argues that Eya acts together with Six1 in mediating metastasis.

In addition to its role in Six-mediated transcription, Eya proteins have recently been shown to play a role in DNA repair. In response to DNA damage, Eya proteins dephosphorylate a phospho-Tyr on histone variant H2AX, and this dephosphorylation is critical for directing cells to the repair instead of apoptotic pathway (Krishnan et al., 2009; Cook et al., 2009). Thus, knockdown of Eya proteins leads to a significant increase in apoptotic cells in response to hypoxia or ionizing radiation (Krishnan et al., 2009; Cook et al., 2009). Currently, about half of all people with cancer are treated with radiation therapy, either alone or with other cancer treatment, to kill cancer cells and reduce tumor burden. Selectively sensitizing tumor tissue by engaging the apoptotic program of a cell is of great interest to the field of radiation oncology (Bernier et al., 2004). It is foreseeable that inhibitors of Eya's phosphatase activity may greatly increase the efficiency of radiation therapy, or of any DNA damaging related therapy (many cancer therapies use a combination of both), in cancers that are known to express Eya, including breast cancers (Farabaugh et al., 2011), Wilms' tumor (Li et al., 2002), ovarian carcinomas (Zhang et al., 2005). Thus, identification of such inhibitors is highly desirable.

### SUMMARY OF THE INVENTION

Thus, in accordance with the present invention, there is provided a method of treating cancer in a subject comprising administering to said subject an effective amount of compound that inhibits EYA2 phosphatase activity, said compound having formula:

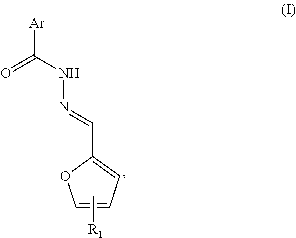

wherein:

Ar is aryl_{(C≦12)}, heteroaryl_{(C≦12)}, or a substituted version of any of these groups;

R₁ is:
hydrogen, hydroxy, amino or mercapto; oralkyl_{(C≦12)}, aralkyl_{(C≦12)}, heteroaralkyl_{(C≦12)}, acyl_{(C≦12)}, alkoxy_{(C≦12)}, aryloxy_{(C≦12)}, aralkoxy_{(C≦12)}, —CH₂-aralkoxy_{(C≦12)}, heteroaryloxy_{(C≦12)}, heteroaralkoxy_{(C≦12)}, acyloxy_{(C≦12)}, alkylamino_{(C≦12)}, dialkylamino_{(C≦12)}, arylamino_{(C≦12)}, aralkylamino_{(C≦12)}, heteroarylamino_{(C≦12)}, heteroaralkylamino_{(C≦12)}, amido_{(C≦12)}, alkylthio_{(C≦12)}, arylthio_{(C≦12)}, aralkylthio_{(C≦12)}, heteroarylthio_{(C≦12)}, heteroaralkylthio_{(C≦12)}, acylthio_{(C≦12)}, or a substituted version of any of these groups;
   or a pharmaceutically acceptable salt or tautomer thereof. In one embodiment, Ar may be aryl_{(C≦12)} or substituted aryl_{(C≦12)}. More particularly, Ar may be aryl_{(C≦8)}, such as phenyl or methylphenyl. Ar may alternatively be substituted aryl_{(C≦8)}, such as flurophenyl, chlorophenyl, bromophenyl or nitrophenyl. Ar may also be heteroaryl_{(C≦12)} or substituted heteroaryl_{(C≦12)}. In particular, the compound may be:

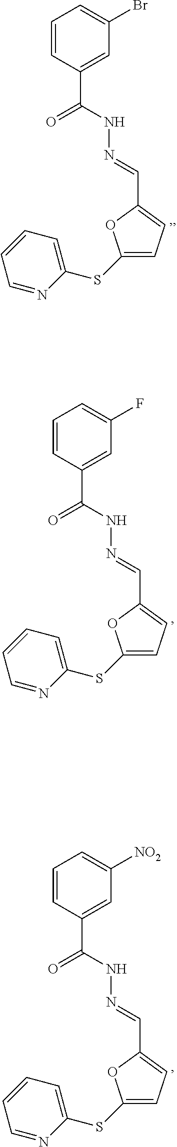 [SMILES: O=C(N/N=C/c1ccc(Sc2ccccn2)o1)c1cccc(Br)c1 ; O=C(N/N=C/c1ccc(Sc2ccccn2)o1)c1cccc(F)c1 ; O=C(N/N=C/c1ccc(Sc2ccccn2)o1)c1cccc([N+](=O)[O-])c1]

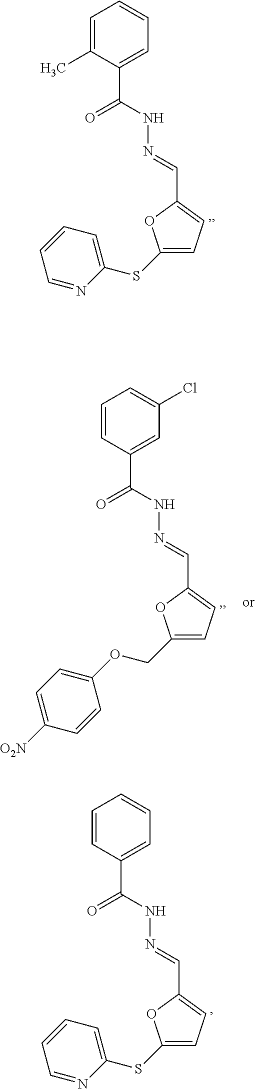 [SMILES: Cc1ccccc1C(=O)N/N=C/c1ccc(Sc2ccccn2)o1 ; O=C(N/N=C/c1ccc(COc2ccc([N+](=O)[O-])cc2)o1)c1cccc(Cl)c1 ; O=C(N/N=C/c1ccc(Sc2ccccn2)o1)c1ccccc1]

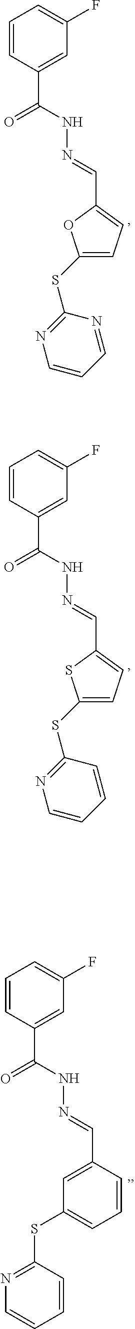 [SMILES: O=C(N/N=C/c1ccc(Sc2ncccn2)o1)c1cccc(F)c1 ; O=C(N/N=C/c1ccc(Sc2ccccn2)s1)c1cccc(F)c1 ; O=C(N/N=C/c1cccc(Sc2ccccn2)c1)c1cccc(F)c1]

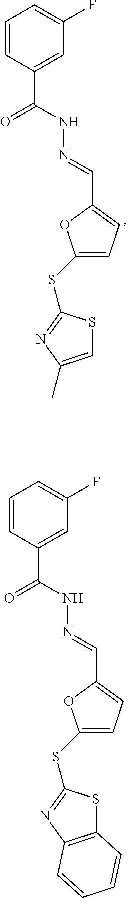 [SMILES: Cc1csc(Sc2ccc(/C=N/NC(=O)c3cccc(F)c3)o2)n1 ; O=C(N/N=C/c1ccc(Sc2nc3ccccc3s2)o1)c1cccc(F)c1]

or a pharmaceutically acceptable salt or tautomer thereof.

In another embodiment, there is provided a method of treating cancer in a subject comprising administering to said subject an effective amount of compound that inhibits EYA2 phosphatase activity, said compound having formula:

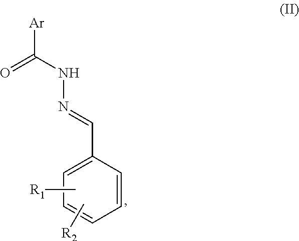

wherein:

Ar is aryl_{(C≦12)}, heteroaryl_{(C≦12)}, or a substituted version of any of these groups;

R₁ and R₂ are each independently:
hydrogen, hydroxy, amino, mercapto, cyano or nitro; or alkyl_{(C≦12)}, aralkyl_{(C≦12)}, heteroaralkyl_{(C≦12)}, acyl_{(C≦12)}, alkoxy_{(C≦12)}, aryloxy_{(C≦12)}, aralkoxy_{(C≦12)}, —CH₂-aralkoxy_{(C≦12)}, heteroaryloxy_{(C≦12)}, heteroaralkoxy_{(C≦12)}, acyloxy_{(C≦12)}, alkylamino_{(C≦12)}, dialkylamino_{(C≦12)}, arylamino_{(C≦12)}, aralkyl-amino_{(C≦12)}, heteroarylamino_{(C≦12)}, heteroaralkylamino_{(C≦12)}, amido_{(C≦12)}, alkylthio_{(C≦12)}, arylthio_{(C≦12)}, aralkylthio_{(C≦12)}, heteroarylthio_{(C≦12)}, heteroaralkylthio_{(C≦12)}, acylthio_{(C≦12)}, or a substituted version of any of these groups;
   or a pharmaceutically acceptable salt or tautomer thereof. Ar may be aryl_{(C≦12)} or substituted aryl_{(C≦12)}, more particularly Ar may be aryl_{(C≦8)}, such as phenyl. Ar may also be substituted aryl_{(C≦8)}, such as fluorophenyl. Ar can also be heteroaryl_{(C≦12)} or substituted heteroaryl_{(C≦12)}, such as substituted heteroaryl_{(C≦8)}, and more particularly 1-N-methyl,4-chloropyrazol-3-yl. In particular, the compound may be:

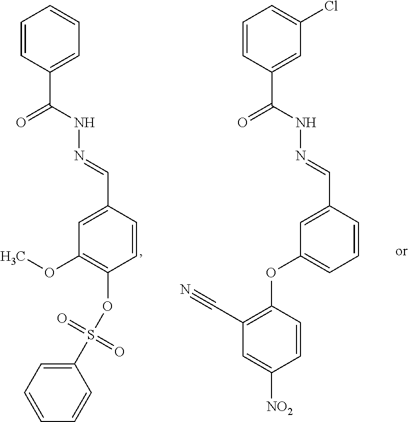 [SMILES: COc1cc(/C=N/NC(=O)c2ccccc2)ccc1OS(=O)(=O)c1ccccc1 ; N#Cc1cc([N+](=O)[O-])ccc1Oc1cccc(/C=N/NC(=O)c2cccc(Cl)c2)c1]

or a pharmaceutically acceptable salt or tautomer thereof.

In yet another embodiment, there is provided a method of treating cancer in a subject comprising administering to said subject an effective amount of compound that inhibits EYA2 phosphatase activity, said compound selected from the following:

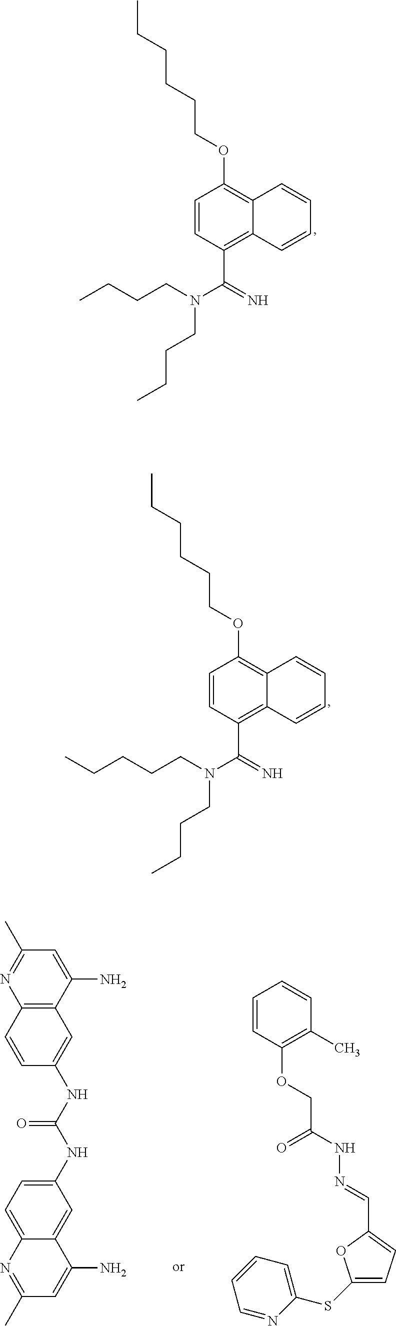 [SMILES: CCCCCCOc1ccc(C(=N)N(CCCC)CCCC)c2ccccc12 ; CCCCCCOc1ccc(C(=N)N(CCCC)CCCCC)c2ccccc12 ; Cc1cc(N)c2cc(NC(=O)Nc3ccc4nc(C)cc(N)c4c3)ccc2n1 ; "or" ; Cc1ccccc1OCC(=O)N/N=C/c1ccc(Sc2ccccn2)o1]

or a pharmaceutically acceptable salt or tautomer of any of the above formulas.

In still yet another embodiment, there is provided a method of treating cancer in a subject comprising administering to said subject an effective amount of compound that inhibits EYA2 phosphatase activity, said compound selected from the following:

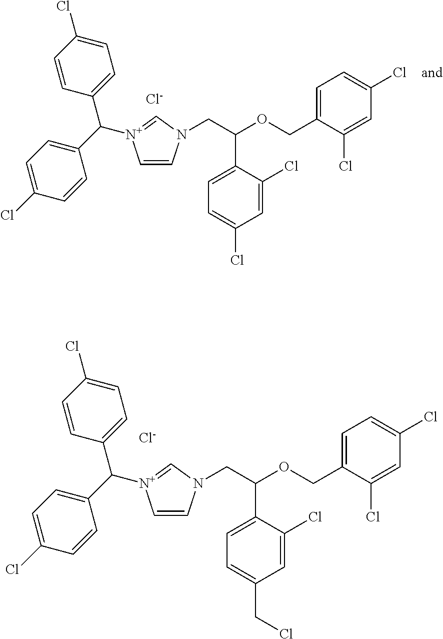 [SMILES: Clc1ccc(C(c2ccc(Cl)cc2)[n+]2ccn(CC(OCc3ccc(Cl)cc3Cl)c3ccc(Cl)cc3Cl)c2)cc1 ; [Cl-] ; "and" ; ClCc1ccc(C(Cn2cc[n+](C(c3ccc(Cl)cc3)c3ccc(Cl)cc3)c2)OCc2ccc(Cl)cc2Cl)c(Cl)c1 ; [Cl-]]

or a salt thereof having either of the above cations combined with a different pharmaceutically acceptable anion.

In accordance with any of the embodiments above, the compound may be administered topically, intravenously, intradermally, intraarterially, intraperitoneally, intralesionally, intracranially, intraarticularly, intraprostaticaly, intrapleurally, intratracheally, intraocularly, intranasally, intravitreally, intravaginally, intrarectally, intramuscularly, subcutaneously, subconjunctivally, intravesicularly, mucosally, intrapericardially, intraumbilically, orally, intratumorally, by inhalation, by injection, by infusion, by continuous infusion, by localized perfusion bathing target cells directly, via a catheter, or via a lavage. The compound may be administered more than once. The compound may further be administered in combination with a second cancer therapy, such as radiotherapy, immunotherapy, chemotherapy, hormonal therapy or gene therapy. The cancer may be breast cancer, lung cancer, prostate cancer, ovarian cancer, brain cancer, liver cancer, cervical cancer, colorectal cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia, and in particular, a cancer that overexpress EYA2.

In a further embodiment, there is provided a method of inhibiting EYA2 phosphatase activity in a cell, said compound having the formula:

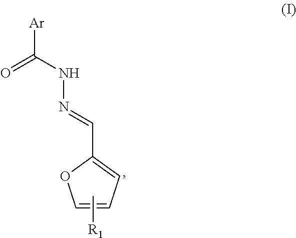

wherein:

Ar is aryl_{(C≦12)}, heteroaryl_{(C≦12)}, or a substituted version of any of these groups;

R₁ is:
hydrogen, hydroxy, amino or mercapto; or alkyl_{(C≦12)}, aralkyl_{(C≦12)}, heteroaralkyl_{(C≦12)}, acyl_{(C≦12)}, alkoxy_{(C≦12)}, aryloxy_{(C≦12)}, aralkoxy_{(C≦12)}, —CH₂-aralkoxy_{(C≦12)}, heteroaryloxy_{(C≦12)}, heteroaralkoxy_{(C≦12)}, acyloxy_{(C≦12)}, alkylamino_{(C≦12)}, dialkylamino_{(C≦12)}, arylamino_{(C≦12)}, aralkylamino_{(C≦12)}, heteroarylamino_{(C≦12)}, heteroaralkylamino_{(C≦12)}, amido_{(C≦12)}, alkylthio_{(C≦12)}, arylthio_{(C≦12)}, aralkylthio_{(C≦12)}, heteroarylthio_{(C≦12)}, heteroaralkylthio_{(C≦12)}, acylthio_{(C≦12)}, or a substituted version of any of these groups;
   or a pharmaceutically acceptable salt or tautomer thereof. Ar may be aryl_{(C≦12)} or substituted aryl_{(C≦12)}, such as aryl_{(C≦8)}, and in particular phenyl or methylphenyl. Ar may also be substituted aryl_{(C≦8)}, such as flurophenyl, chlorophenyl, bromophenyl or nitrophenyl. Ar can also be heteroaryl_{(C≦12)} or substituted heteroaryl_{(C≦12)}. In particular, the compound may be:

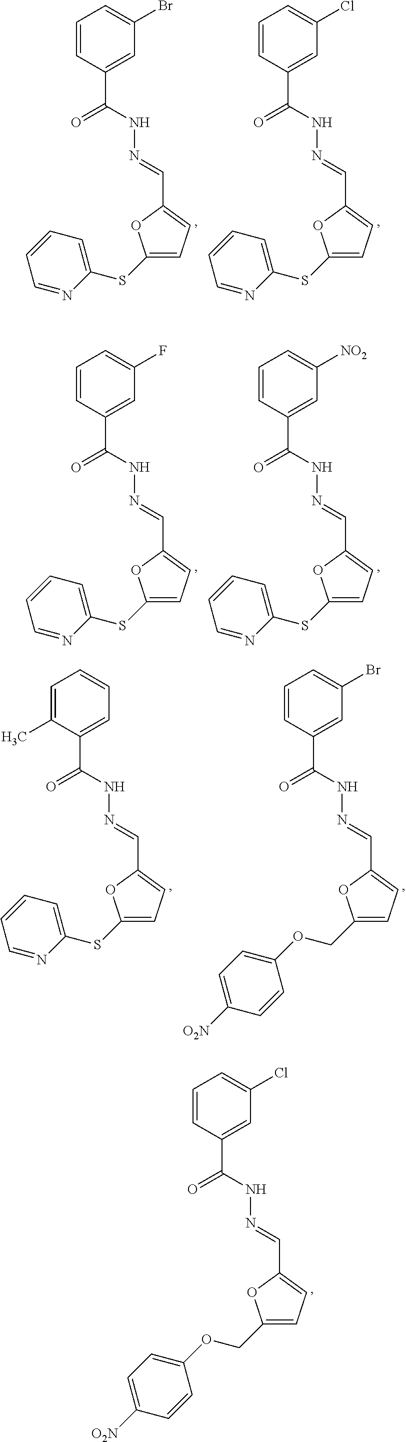

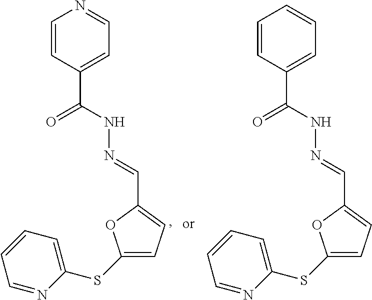 [SMILES: O=C(N/N=C/c1ccc(Sc2ccccn2)o1)c1ccncc1 ; "," ; "or" ; O=C(N/N=C/c1ccc(Sc2ccccn2)o1)c1ccccc1]

or a pharmaceutically acceptable salt or tautomer thereof. The cell may overexpress Eya2.

In yet a further embodiment, there is provided a method of inhibiting EYA2 phosphatase activity in a cell, said compound having the formula:

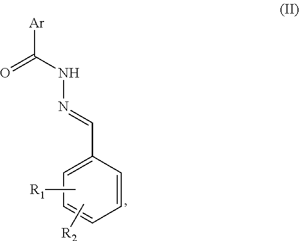

wherein:

Ar is aryl_{(C≦12)}, heteroaryl_{(C≦12)}, or a substituted version of any of these groups;

R₁ and R₂ are each independently:
hydrogen, hydroxy, amino, mercapto, cyano or nitro; or alkyl_{(C≦12)}, aralkyl_{(C≦12)}, heteroaralkyl_{(C≦12)}, acyl_{(C≦12)}, alkoxy_{(C≦12)}, aryloxy_{(C≦12)}, aralkoxy_{(C≦12)}, heteroaryloxy_{(C≦12)}, heteroaralkoxy_{(C≦12)}, acyloxy_{(C≦12)}, alkylamino_{(C≦12)}, dialkylamino_{(C≦12)}, arylamino_{(C≦12)}, aralkylamino_{(C≦12)}, heteroarylamino_{(C≦12)}, heteroaralkylamino_{(C≦12)}, amido_{(C≦12)}, aryl-sulfonyloxy_{(C≦12)}, or a substituted version of any of these groups;
   or a pharmaceutically acceptable salt or tautomer thereof. Ar may be aryl_{(C≦12)} or substituted aryl_{(C≦12)}, such as aryl_{(C≦8)}, and in particular phenyl. Ar may also be substituted aryl_{(C≦8)}, such as chlorophenyl. Ar may further be heteroaryl_{(C≦12)} or substituted heteroaryl_{(C≦12)}, such as substituted heteroaryl_{(C≦8)}, including 1-N-methyl,4-chloropyrazol-3-yl. In particular, the compound is:

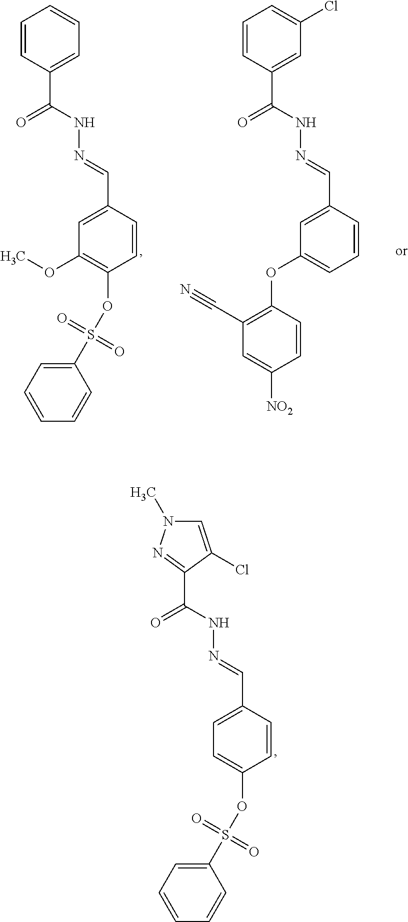 [SMILES: COc1cc(/C=N/NC(=O)c2ccccc2)ccc1OS(=O)(=O)c1ccccc1 ; N#Cc1cc([N+](=O)[O-])ccc1Oc1cccc(/C=N/NC(=O)c2cccc(Cl)c2)c1 ; Cn1cc(Cl)c(C(=O)N/N=C/c2ccc(OS(=O)(=O)c3ccccc3)cc2)n1]

or a pharmaceutically acceptable salt or tautomer thereof. The cell may overexpress Eya2.

Also provided is a method of inhibiting EYA2 phosphatase activity in a cell, said compound selected from the following:

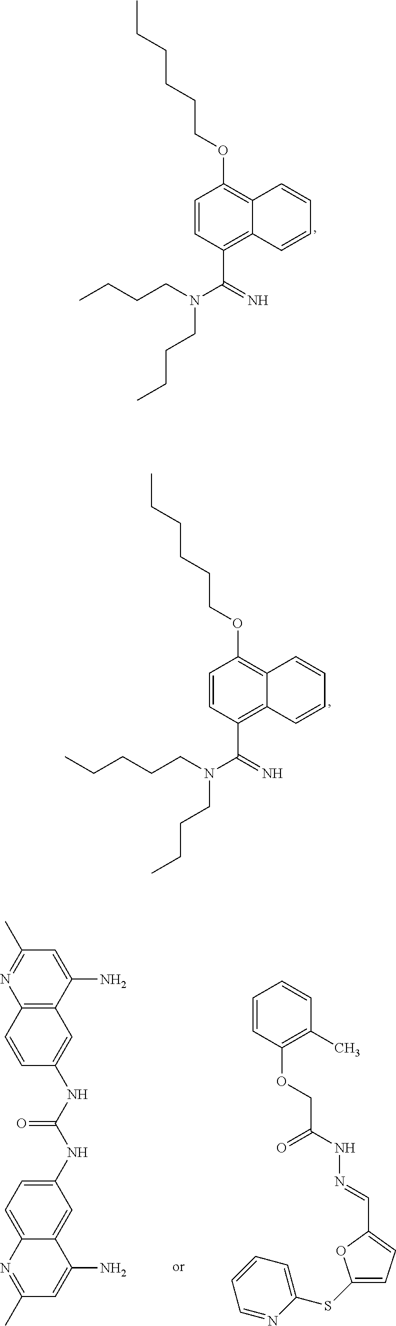 [SMILES: CCCCCCOc1ccc(C(=N)N(CCCC)CCCC)c2ccccc12 ; CCCCCCOc1ccc(C(=N)N(CCCC)CCCCC)c2ccccc12 ; Cc1cc(N)c2cc(NC(=O)Nc3ccc4nc(C)cc(N)c4c3)ccc2n1 ; "or" ; Cc1ccccc1OCC(=O)N/N=C/c1ccc(Sc2ccccn2)o1]

or a pharmaceutically acceptable salt or tautomer of any of the above formulas. The cell may overexpress Eya2.

Another embodiment comprises a method of inhibiting EYA2 phosphatase activity in a cell, said compound selected from the following:

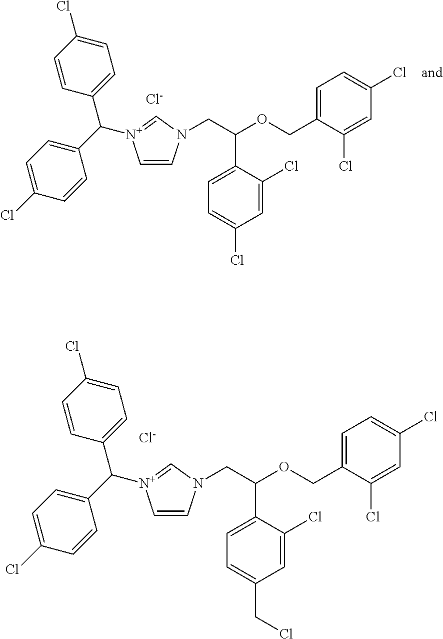 [SMILES: Clc1ccc(C(c2ccc(Cl)cc2)[n+]2ccn(CC(OCc3ccc(Cl)cc3Cl)c3ccc(Cl)cc3Cl)c2)cc1 ; [Cl-] ; "and" ; ClCc1ccc(C(Cn2cc[n+](C(c3ccc(Cl)cc3)c3ccc(Cl)cc3)c2)OCc2ccc(Cl)cc2Cl)c(Cl)c1 ; [Cl-]]

or a salt thereof having either of the above cations combined with a different pharmaceutically acceptable anion. The cell may overexpress Eya2.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these figures in combination with the detailed description of specific embodiments presented herein.

FIGS. 1A-B. Eya2 ED purified from E. coli is an active phosphatase. (FIG. 1A) Enzyme kinetics of Eya2 ED phosphatase activity using the small molecule substrate OMFP. RFU: relative fluorescence unit. (FIG. 1B) EDTA effectively inhibits Eya2 phosphatase activity with an IC₅₀ of 65 μM and can be used as a positive control for inhibition.

FIG. 2. Common phosphatase inhibitors do not significantly inhibit Eya2 ED. Compounds showing inhibition display high IC₅₀ values: 11.3 mM for Na₂MnO₄, 8.2 mM for β-glycerophosphate, 6.6 mM for NaF, and 1.8 mM for Na₃VO₄. Okadaic acid, L-phenylalanine, cyclosporine A, 1,10-phenanthroline, and phenylarsine oxide do not show inhibition at concentrations tested.

FIGS. 3A-E. HTS assay optimization for the Eya2 phosphatase assay using OMFP as a substrate. (FIG. 3A) Scattered plot of a DMSO plate test of the Eya2 phosphatase assay in a 1536-well plate. The wells in column 2 contain 1 mM EGTA that was used as a positive control. All other wells contain DMSO as the negative (no inhibition) control. (FIG. 3B) Z′ and signal/background (S/B) values of a pilot screen using the LOPAC library. (FIG. 3C) Pilot screening revealed three compounds that inhibit Eya2 phosphatase activity. (FIG. 3D) Chemical structures of the three compounds in C. (FIG. 3E) Two pilot screen compounds inhibit Eya2 phosphate activity using pH2AX as a substrate.

FIGS. 4A-D. A new series of Eya2 ED phosphatase inhibitors were identified in the HTS that inhibit Eya2's phosphatase activity. (FIG. 4A) Structures of compounds that demonstrate inhibitory activity in the large scale HTS. (FIG. 4B) Activity of this series was confirmed in 12-point dose response curves. IC₅₀ values of each compound are also listed. (FIG. 4C) Structures of four additional commercially available compounds that Identification of Eya2 phosphatase inhibitors have the same N-arylidenebenzohydrazide core. (FIG. 4D) The four compounds tested display varying levels of inhibition towards Eya2 ED.

FIG. 5. A secondary, malachite green based phosphatase assay using a phospho-H2AX peptide as the substrate confirmed the inhibition of Eya2's phosphatase activity by the best compounds of this class.

FIGS. 6A-F. Identified lead compounds specifically inhibit the Eya2 Tyr phosphatase activity, inhibit Six1-mediated transcriptional activity, but do not significantly inhibit other cellular phosphatases, including the Mg2+-dependent phosphatase PPM1A (FIG. 6A), protein tyrosine phosphatase 1B (PTP1B) (FIG. 6B), and HAD family member Scp1 (FIG. 6C) in an OMFP-based phosphatase assay. FIG. 7. Identified lead compounds inhibit. (FIG. 6D) Six1 and Eya2 together (but not individually) induce transcriptional activation of the MEF3 promoter. (FIG. 6E) Phosphatase-dead Eya2 mutant (D274N) has significantly reduced ability to induce transcriptional activation of the MEF3 promoter. (FIG. 6F) Compounds that effectively inhibit the Eya2 phosphatase in the OMFP assay (MLS000544460 and MLS000585814) also inhibit Six1/Eya2-mediated transcriptional activation of the MEF3 promoter, while a compound that does not significantly inhibit Eya2's phosphatase activity in the OMFP assay (NCGC00241224) does not significantly inhibit Six1/Eya2 mediated transcription. “**” represents p<0.01 and “ns” stands for statistically non-significant. Statistical significance was determined using ANOVA with Dunnett's multiple comparisons on the log transformed ratios of transcriptional activity in the presence of drug versus vehicle control.

FIG. 7. Increasing concentrations of EDTA is required to completely inhibit the Eya2 phosphatase activity with increasing Mg²⁺ concentrations.

FIG. 8. Structures and IC₅₀s of the lead compounds identified from the primary HTS screen.

FIGS. 9A-C. The effect of EDTA and Mg²⁺ on the ED protein thermostability. (FIG. 9A) Increasing EDTA concentrations decrease the protein melting temperatures (Tₘ). (FIG. 9B) Increasing concentrations of Mg²⁺ increase Tₘs. (FIG. 9C) Increasing EDTA concentrations reduce Tₘ without extra Mg²⁺ added to the ED protein sample but have no significant effect in the presence of 5 μM Mg²⁺.

FIGS. 10A-D. The effect of compounds on the Tₘ of ED. (FIG. 10A) Increasing concentrations of compounds generally decrease the Tₘ of ED in the presence of 5 μM Mg²⁺/No EDTA. ΔTₘ indicates the difference between Tₘ at a certain compound concentration and Tₘ for the apo protein (i.e., no compound present). (FIG. 10B) Increasing concentrations of compounds generally increase the Tₘ of ED in the absence of 5 mM EDTA/No extra Mg²⁺. (FIG. 10C) The Tₘ decrease (destabilization) caused by the compounds in the presence of 5 μM Mg²⁺ correlates with the Tₘ increase (stabilization) caused by the compounds in the presence of 5 mM EDTA. (FIG. 10D) The Tₘ increase caused by the compounds in the presence of 5 mM EDTA correlates with their IC₅₀s obtained in the primary screen.

FIGS. 11A-F. The Eva inhibitors have a weak binding affinity to Mg²⁺. (FIG. 11A) Structures of the three most active compounds with potential Mg²⁺ coordination sites shown in red. (FIG. 11B) UV absorption spectrum of compound MLS000544460 in the presence of Mg²⁺. λₘₐₓ shifted from 322 nm to 334 nm with increasing concentrations of Mg²⁺. (FIG. 11C) The λₘₐₓ shift observed in (FIG. 11B) is quantified as a function of Mg²⁺ concentration. (FIG. 11D) The UV absorption spectrum of MLS000544460 in the presence of Na⁺. λₘₐₓ remained at 323 nm when titrated with Na⁺. (FIG. 11E) UV absorption spectrum of the low activity analog MLS000585814 in the presence of Mg²⁺. λₘₐₓ shifted from 316 nm (no Mg²⁺) to 327 nm (50 mM Mg²⁺). (FIG. 11F) The λₘₐₓ shift observed in (FIG. 11E) is quantified as a function of Mg²⁺ concentration.

FIG. 12. ¹H NMR spectra of MLS000544460 in CD₃CN in the absence of any metal ions, in the presence of Mg²⁺, and in the presence of Na⁺. The hydrazide proton peak is indicated with a box.

FIG. 13. The activity of lead compounds in dilution assays. 1 nM of enzyme was incubated for 70 (1 hour) or 190 (3 hours) minutes with several concentrations of compound MLS000544460 (0.4, 4 and 40 μM) prior to the addition of OMFP. For the 100× conditions, 100 nM of enzyme was incubated without inhibitor (no compound column) or with 40 μM of compound for 10 minutes and then the reaction was diluted 100 times, followed by additional incubation for 1 hour or 3 hours prior to the addition of OMFP. The fluorescent signal was measure 20 minutes after substrate addition.

FIG. 14. Enzymatic kinetic experiments indicate that the compounds are mixed-mode inhibitors.

FIG. 15. Proposed binding model of inhibitor MLS000544460 in the active site of Eya2.

### DESCRIPTION OF ILLUSTRATIVE EMBODIMENTS

Cancer shares many common properties with normal development. During normal development of an organ, genes are activated to stimulate the proliferation and survival of progenitor cells, as well as to stimulate migration, invasion, and neovascularization. These genes are usually down-regulated once organ development is completed. In cancer, the same genes are often re-activated, stimulating inappropriate proliferation, survival, migration, invasion, and neovascularization. The homeobox gene and transcription factor Six1 plays a critical role in the development of numerous organs through its ability to increase proliferation and decrease apoptosis, leading to an expansion of progenitor cell populations (Kawakami et al., 2000; Xu et al., 2003; Zheng et al., 2003; Laclef et al., 2003a; Laclef et al., 2003b; Ozaki et al., 2004). Six1 expression is undetectable or low in normal adult breast tissue, but it is over-expressed in 50% primary breast tumors and 90% metastatic lesions (Coletta et al., 2004; Reichenberger et al., 2005). Six1 over-expression is linked to enhanced cellular proliferation, transformation, increased tumor volume, epithelial to mesenchymal transition, and metastasis in breast cancer (Coletta et al., 2004; Coletta et al., 2008). Further, epithelial-to-mesenchymal transition (EMT) is heavily associated with metastasis. The relocalization of Ecad and β-catenin is associated with EMT.

Data shows that overexpression of Six1 can transform immortalized, but otherwise normal, mammary epithelial cells, leading to highly aggressive tumors in vivo. Furthermore, data in which Six1 was overexpressed specifically in the mammary gland show that Six1 induces hyperplasia and tumor formation in vivo. Together, these data indicated that Six1 is involved in tumor initiation. The higher percentage of Six1 over-expression in metastatic breast lesions indicates that it may also play an important role in breast tumor metastasis. Examination of more than 130 breast cancers demonstrates that Six1 overexpression correlates significantly with positive lymph node status (p<0.05). Six1 overexpression in tumorigenic, but non-metastatic MCF7 cells leads to both lymphatic and bone metastasis in a mouse orthotopic breast cancer model. Six1 expression leads to expansion of CD24+/CD29^{hi} mammary stem/progenitor cells in transgenic mice, and to the expansion of CD44+/CD24lo cancer stem cells in MCF7 mammary carcinoma cells. These studies indicate that Six1 is a powerful oncogene that can not only induce tumorigenesis, but can also cause metastasis.

Furthermore, Six1 is overexpressed in both ovarian carcinoma and hepatacellular carcinoma, and its expression correlates with worsened survival in both cancer types (Ng et al., 2006). Six1 is also overexpressed in rhabdomyosarcomas where its expression correlates with advanced tumor stage and where it is shown to be critical for metastasis (Li et al., 2002; Khan et al., 1999; Yu et al., 2004). Six1 is amplified in a small percentage (about 5%) of human breast cancers and is overexpressed in Wilms' Tumor (Li et al., 2002). These data suggest that Six1 plays a role in the progression of many tumor types. Because Six1 is overexpressed in multiple cancers, and because it is an embryonic gene whose expression is absent in most differentiated adult tissues, it is an ideal drug target whose inactivation will inhibit tumor cell proliferation, survival, and metastasis with limited side effects. Importantly, RNA interference against Six1 decreases cancer cell proliferation and metastases in several different models of cancer. The inventors identified several miRNAs that target the 3′ UTR of the Six1 gene and down-regulate Six1 expression. They also identified miRNAs that correlate with alterations in Six1 expression, and thus can be used to identify Six1-involved cancers, as well as prove diagnostic of cancer in their own right.

Although it has traditionally been difficult to identify specific phosphatase inhibitors, the fact that Eya proteins belong to the HAD family of protein phosphatases, that use an Asp instead of the more commonly used Cys as their active site residue, provides for a unique opportunity to potentially identify specific Eya phosphatase inhibitors. Here the inventors report here the identification and characterization of a previously unknown chemical series that specifically inhibit the Eya2 phosphatase. These compounds also inhibit Six1-mediated transcription in a MEF3-promoter luciferase assay, suggesting that the compounds can enter the cell and can inhibit Six1/Eya2 transcriptional activity. These novel compounds can be used by the research community as chemical probes to further study the function of Eya's phosphatase activity and its role in Six1-mediated breast tumorigenesis and metastasis. There is also the exciting possibility of developing this series of compounds into potential anti-cancer drugs in the future.

### I. DEFINITIONS

When used in the context of a chemical group, “hydrogen” means —H; “hydroxy” means —OH; “oxo” means=O; “halo” means independently —F, —Cl, —Br or —I; “amino” means —NH₂ (see below for definitions of groups containing the term amino, e.g., alkylamino); “hydroxyamino” means —NHOH; “nitro” means —NO₂; imino means=NH (see below for definitions of groups containing the term imino, e.g., alkylimino); “cyano” means —CN; “azido” means —N₃; in a monovalent context “phosphate” means —OP(O)(OH)₂ or a deprotonated form thereof; in a divalent context “phosphate” means —OP(O(OH)O— or a deprotonated form thereof; “mercapto” means —SH; “thio” means=S; “thioether” means —S—; “sulfonamido” means —NHS(O)₂— (see below for definitions of groups containing the term sulfonamido, e.g., alkylsulfonamido); “sulfonyl” means —S(O)₂— (see below for definitions of groups containing the term sulfonyl, e.g., alkylsulfonyl); “sulfinyl” means —S(O)— (see below for definitions of groups containing the term sulfinyl, e.g., alkylsulfinyl); and “silyl” means —SiH₃ (see below for definitions of group(s) containing the term silyl, e.g., alkylsilyl).

The symbol “-” means a single bond, “=” means a double bond, and “≡” means triple bond. The symbol “- - - -” represents an optional bond, which if present is either single or double. The symbol “ [symbol] ” represents a single bond or a double bond. The symbol “ [symbol] ”, when drawn perpendicularly across a bond indicates a point of attachment of the group. It is noted that the point of attachment is typically only identified in this manner for larger groups in order to assist the reader in rapidly and unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the conformation is unknown (e.g., either R or 5), the geometry is unknown (e.g., either E or Z) or the compound is present as mixture of conformation or geometries (e.g., a 50%/50% mixture).

When a group “R” is depicted as a “floating group” on a ring system, for example, in the formula:

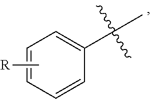

then R may replace any hydrogen atom attached to any of the ring atoms, including a depicted, implied, or expressly defined hydrogen, so long as a stable structure is formed.

When a group “R” is depicted as a “floating group” on a fused ring system, as for example in the formula:

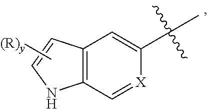

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

When y is 2 and “(R)_{y}” is depicted as a floating group on a ring system having one or more ring atoms having two replaceable hydrogens, e.g., a saturated ring carbon, as for example in the formula:

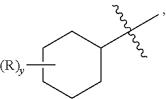

then each of the two R groups can reside on the same or a different ring atom. For example, when R is methyl and both R groups are attached to the same ring atom, a geminal dimethyl group results. Where specifically provided for, two R groups may be taken together to form a divalent group, such as one of the divalent groups further defined below. When such a divalent group is attached to the same ring atom, a spirocyclic ring structure will result.

When the point of attachment is depicted as “floating”, for example, in the formula:

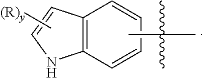

then the point of attachment may replace any replaceable hydrogen atom on any of the ring atoms of either of the fused rings unless specified otherwise.

In the case of a double-bonded R group (e.g., oxo, imino, thio, alkylidene, etc.), any pair of implicit or explicit hydrogen atoms attached to one ring atom can be replaced by the R group. This concept is exemplified below:

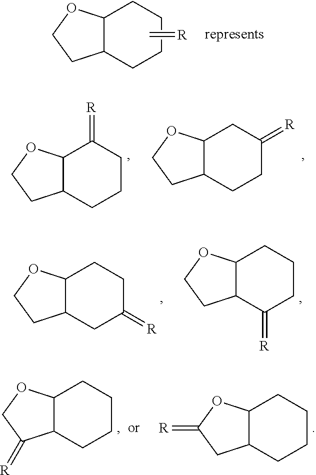

For the groups and classes below, the following parenthetical subscripts further define the group/class as follows: “(Cn)” defines the exact number (n) of carbon atoms in the group/class. “(C≦n)” defines the maximum number (n) of carbon atoms that can be in the group/class, with the minimum number as small as possible for the group in question, e.g., it is understood that the minimum number of carbon atoms in the group “alkenyl_{(C≦8)}” or the class “alkene_{(C≦8)}” is two. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3 to 10 carbon atoms). (Cn-n′) defines both the minimum (n) and maximum number (n′) of carbon atoms in the group. Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3 to 10 carbon atoms)).

The term “alkyl” when used without the “substituted” modifier refers to a non-aromatic monovalent group with a saturated carbon atom as the point of attachment, a linear or branched, cyclo, cyclic or acyclic structure, no carbon-carbon double or triple bonds, and no atoms other than carbon and hydrogen. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “substituted alkyl” refers to a non-aromatic monovalent group with a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Cl, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂C(O)CH₃, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃.

The term “alkanediyl” when used without the “substituted” modifier refers to a non-aromatic divalent group, wherein the alkanediyl group is attached with two σ-bonds, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, —CH₂CH₂CH₂—, and Error! Objects cannot be created from editing field codes., are non-limiting examples of alkanediyl groups. The term “substituted alkanediyl” refers to a non-aromatic monovalent group, wherein the alkynediyl group is attached with two σ-bonds, with one or two saturated carbon atom(s) as the point(s) of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkanediyl groups: —CH(F)—, —CF₂—, —CH(Cl)—, —CH(OH)—, —CH(OCH₃)—, and —CH₂CH(Cl)—.

The term “alkane” when used without the “substituted” modifier refers to a non-aromatic hydrocarbon consisting only of saturated carbon atoms and hydrogen and having a linear or branched, cyclo, cyclic or acyclic structure. Thus, as used herein cycloalkane is a subset of alkane. The compounds CH₄ (methane), CH₃CH₃ (ethane), CH₃CH₂CH₃ (propane), (CH₂)₃ (cyclopropane), CH₃CH₂CH₂CH₃ (n-butane), and CH₃CH(CH₃)CH₃ (isobutane), are non-limiting examples of alkanes. A “substituted alkane” differs from an alkane in that it also comprises at least one atom independently selected from the group consisting of N, O, F, Cl, Br, I, Si, P, and S. The following compounds are non-limiting examples of substituted alkanes: CH₃OH, CH₃Cl, nitromethane, CF₄, CH₃OCH₃ and CH₃CH₂NH₂.

The term “alkenyl” when used without the “substituted” modifier refers to a monovalent group with a nonaromatic carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “substituted alkenyl” refers to a monovalent group with a nonaromatic carbon atom as the point of attachment, at least one nonaromatic carbon-carbon double bond, no carbon-carbon triple bonds, a linear or branched, cyclo, cyclic or acyclic structure, and at least one atom independently selected from the group consisting of N, O, F, Cl, Br, I, Si, P, and S. The groups, —CH═CHF, —CH═CHCl and —CH═CHBr, are non-limiting examples of substituted alkenyl groups.

The term “alkenediyl” when used without the “substituted” modifier refers to a non-aromatic divalent group, wherein the alkenediyl group is attached with two σ-bonds, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—, —CH═CHCH₂—, and

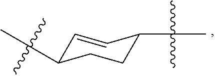 [SMILES: *C1C=CC(*)CC1]

are non-limiting examples of alkenediyl groups. The term “substituted alkenediyl” refers to a non-aromatic divalent group, wherein the alkenediyl group is attached with two σ-bonds, with two carbon atoms as points of attachment, a linear or branched, cyclo, cyclic or acyclic structure, at least one nonaromatic carbon-carbon double bond, no carbon-carbon triple bonds, and at least one atom independently selected from the group consisting of N, O, F, Cl, Br, I, Si, P, and S. The following groups are non-limiting examples of substituted alkenediyl groups: —CF═CH—, —C(OH)═CH—, and —CH₂CH═C(Cl)—.

The term “alkene” when used without the “substituted” modifier refers to a non-aromatic hydrocarbon having at least one carbon-carbon double bond and a linear or branched, cyclo, cyclic or acyclic structure. Thus, as used herein, cycloalkene is a subset of alkene. The compounds C₂H₄ (ethylene), CH₃CH═CH₂ (propene) and cylcohexene are non-limiting examples of alkenes. A “substituted alkene” differs from an alkene in that it also comprises at least one atom independently selected from the group consisting of N, O, F, Cl, Br, I, Si, P, and S.

The term “alkynyl” when used without the “substituted” modifier refers to a monovalent group with a nonaromatic carbon atom as the point of attachment, a linear or branched, cyclo, cyclic or acyclic structure, at least one carbon-carbon triple bond, and no atoms other than carbon and hydrogen. The groups, —CCH, —CCCH₃, —CCC₆H₅ and —CH₂CCCH₃, are non-limiting examples of alkynyl groups. The term “substituted alkynyl” refers to a monovalent group with a nonaromatic carbon atom as the point of attachment and at least one carbon-carbon triple bond, a linear or branched, cyclo, cyclic or acyclic structure, and at least one atom independently selected from the group consisting of N, O, F, Cl, Br, I, Si, P, and S. The group, —C≡CSi(CH₃)₃, is a non-limiting example of a substituted alkynyl group.

The term “alkynediyl” when used without the “substituted” modifier refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and no atoms other than carbon and hydrogen. The groups, —C≡C—, —C≡CCH₂—, and —C≡CCH(CH₃)— are non-limiting examples of alkynediyl groups. The term “substituted alkynediyl” refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and at least one atom independently selected from the group consisting of N, O, F, Cl, Br, I, Si, P, and S. The groups —C≡CCFH— and —C≡CHCH(Cl)— are non-limiting examples of substituted alkynediyl groups.

The term “alkyne” when used without the “substituted” modifier refers to a non-aromatic hydrocarbon having at least one carbon-carbon triple bond and a linear or branched, cyclo, cyclic or acyclic structure. Thus, as used herein, cycloalkene is a subset of alkene. The compounds C₂H₂ (acetylene), CH₃C≡CH (propene) and cylcooctyne are non-limiting examples of alkenes. A “substituted alkene” differs from an alkene in that it also comprises at least one atom independently selected from the group consisting of N, O, F, Cl, Br, I, Si, P, and S.

The term “aryl” when used without the “substituted” modifier refers to a monovalent group with an aromatic carbon atom as the point of attachment, said carbon atom forming part of one or more six-membered aromatic ring structure(s) wherein the ring atoms are all carbon, and wherein the monovalent group consists of no atoms other than carbon and hydrogen. Non-limiting examples of aryl groups include phenyl (Ph), methylphenyl, (dimethyl)phenyl, —C₆H₄CH₂CH₃ (ethylphenyl), —C₆H₄CH₂CH₂CH₃ (propylphenyl), —C₆H₄CH(CH₃)₂, —C₆H₄CH(CH₂)₂, —C₆H₃(CH₃)CH₂CH₃ (methylethylphenyl), —C₆H₄CH═CH₂ (vinylphenyl), —C₆H₄CH═CHCH₃, —C₆H₄CCH, —C₆H₄CCCH₃, naphthyl, and the monovalent group derived from biphenyl. The term “substituted aryl” refers to a monovalent group with an aromatic carbon atom as the point of attachment, said carbon atom forming part of one or more six-membered aromatic ring structure(s) wherein the ring atoms are all carbon, and wherein the monovalent group further has at least one atom independently selected from the group consisting of N, O, F, Cl, Br, I, Si, P, and S. Non-limiting examples of substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, and —C₆H₄CON(CH₃)₂.

The term “arenediyl” when used without the “substituted” modifier refers to a divalent group, wherein the arenediyl group is attached with two σ-bonds, with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. Non-limiting examples of arenediyl groups include:

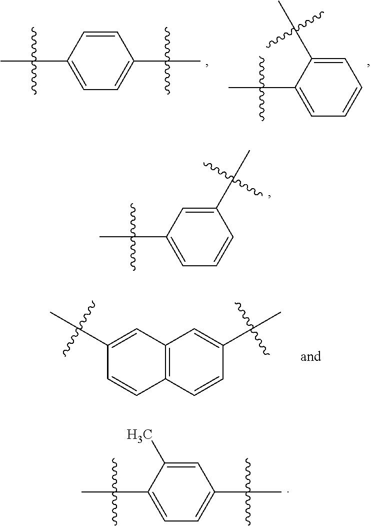

The term “substituted arenediyl” refers to a divalent group, wherein the arenediyl group is attached with two σ-bonds, with two aromatic carbon atoms as points of attachment, said carbon atoms forming part of one or more six-membered aromatic rings structure(s), wherein the ring atoms are carbon, and wherein the divalent group further has at least one atom independently selected from the group consisting of N, O, F, Cl, Br, I, Si, P, and S.

The term “arene” when used without the “substituted” modifier refers to an hydrocarbon having at least one six-membered aromatic ring. One or more alkyl, alkenyl or alkynyl groups may be optionally attached to this ring. Also this ring may optionally be fused with other rings, including non-aromatic rings. Benzene, toluene, naphthalene, and biphenyl are non-limiting examples of arenes. A “substituted arene” differs from an arene in that it also comprises at least one atom independently selected from the group consisting of N, O, F, Cl, Br, I, Si, P, and S. Phenol and nitrobenzene are non-limiting examples of substituted arenes.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn), 1-phenyl-ethyl, 2-phenyl-ethyl, indenyl and 2,3-dihydro-indenyl, provided that indenyl and 2,3-dihydro-indenyl are only examples of aralkyl in so far as the point of attachment in each case is one of the saturated carbon atoms. When the term “aralkyl” is used with the “substituted” modifier, either one or both the alkanediyl and the aryl is substituted. Non-limiting examples of substituted aralkyls are: (3-chlorophenyl)-methyl, 2-oxo-2-phenyl-ethyl (phenylcarbonylmethyl), 2-chloro-2-phenyl-ethyl, chromanyl where the point of attachment is one of the saturated carbon atoms, and tetrahydroquinolinyl where the point of attachment is one of the saturated atoms.

The term “heteroaryl” when used without the “substituted” modifier refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group consists of no atoms other than carbon, hydrogen, aromatic nitrogen, aromatic oxygen and aromatic sulfur. Non-limiting examples of aryl groups include acridinyl, furanyl, imidazoimidazolyl, imidazopyrazolyl, imidazopyridinyl, imidazopyrimidinyl, indolyl, indazolinyl, methylpyridyl, oxazolyl, phenylimidazolyl, pyridyl, pyrrolyl, pyrimidyl, pyrazinyl, quinolyl, quinazolyl, quinoxalinyl, tetrahydroquinolinyl, thienyl, triazinyl, pyrrolopyridinyl, pyrrolopyrimidinyl, pyrrolopyrazinyl, pyrrolotriazinyl, pyrroloimidazolyl, chromenyl (where the point of attachment is one of the aromatic atoms), and chromanyl (where the point of attachment is one of the aromatic atoms). The term “substituted heteroaryl” refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group further has at least one atom independently selected from the group consisting of non-aromatic nitrogen, non-aromatic oxygen, non aromatic sulfur F, Cl, Br, I, Si, and P.

The term “heteroarenediyl” when used without the “substituted” modifier refers to a divalent group, wherein the heteroarenediyl group is attached with two σ-bonds, with an aromatic carbon atom or nitrogen atom as the point of attachment, said carbon atom or nitrogen atom forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. Non-limiting examples of heteroarenediyl groups include:

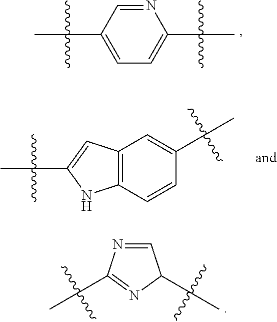

The term “substituted heteroarenediyl” refers to a divalent group, wherein the heteroarenediyl group is attached with two σ-bonds, with an aromatic carbon atom or nitrogen atom as points of attachment, said carbon atom or nitrogen atom forming part of one or more six-membered aromatic ring structure(s), wherein at least one of the ring atoms is nitrogen, oxygen or sulfur, and wherein the divalent group further has at least one atom independently selected from the group consisting of non-aromatic nitrogen, non-aromatic oxygen, non aromatic sulfur F, Cl, Br, I, Si, and P.

The term “heteroaralkyl” when used without the “substituted” modifier refers to the monovalent group -alkanediyl-heteroaryl, in which the terms alkanediyl and heteroaryl are each used in a manner consistent with the definitions provided above. Non-limiting examples of aralkyls are: pyridylmethyl, and thienylmethyl. When the term “heteroaralkyl” is used with the “substituted” modifier, either one or both the alkanediyl and the heteroaryl is substituted.

The term “acyl” when used without the “substituted” modifier refers to a monovalent group with a carbon atom of a carbonyl group as the point of attachment, further having a linear or branched, cyclo, cyclic or acyclic structure, further having no additional atoms that are not carbon or hydrogen, beyond the oxygen atom of the carbonyl group. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)C₆H₄CH₂CH₃, —COC₆H₃(CH₃)₂, and —C(O)CH₂C₆H₅, are non-limiting examples of acyl groups. The term “acyl” therefore encompasses, but is not limited to groups sometimes referred to as “alkyl carbonyl” and “aryl carbonyl” groups. The term “substituted acyl” refers to a monovalent group with a carbon atom of a carbonyl group as the point of attachment, further having a linear or branched, cyclo, cyclic or acyclic structure, further having at least one atom, in addition to the oxygen of the carbonyl group, independently selected from the group consisting of N, O, F, Cl, Br, I, Si, P, and S. The groups, —C(O)CH₂CF₃, —CO₂H (carboxyl), —CO₂CH₃ (methylcarboxyl), —CO₂CH₂CH₃, —CO₂CH₂CH₂CH₃, —CO₂C₆H₅, —CO₂CH(CH₃)₂, —CO₂CH(CH₂)₂, —C(O)NH₂ (carbamoyl), —C(O)NHCH₃, —C(O)NHCH₂CH₃, —CONHCH(CH₃)₂, —CONHCH(CH₂)₂, —CON(CH₃)₂, —CONHCH₂CF₃, —CO-pyridyl, —CO-imidazoyl, and —C(O)N₃, are non-limiting examples of substituted acyl groups. The term “substituted acyl” encompasses, but is not limited to, “heteroaryl carbonyl” groups.

The term “alkylidene” when used without the “substituted” modifier refers to the divalent group ═CRR′, wherein the alkylidene group is attached with one σ-bond and one π-bond, in which R and R′ are independently hydrogen, alkyl, or R and R′ are taken together to represent alkanediyl. Non-limiting examples of alkylidene groups include: ═CH₂, ═CH(CH₂CH₃), and ═C(CH₃)₂. The term “substituted alkylidene” refers to the group═CRR′, wherein the alkylidene group is attached with one σ-bond and one π-bond, in which R and R′ are independently hydrogen, alkyl, substituted alkyl, or R and R′ are taken together to represent a substituted alkanediyl, provided that either one of R and R′ is a substituted alkyl or R and R′ are taken together to represent a substituted alkanediyl.

The term “alkoxy” when used without the “substituted” modifier refers to the group —OR, in which R is an alkyl, as that term is defined above. Non-limiting examples of alkoxy groups include: —OCH₃, —OCH₂CH₃, —OCH₂CH₂CH₃, —OCH(CH₃)₂, —OCH(CH₂)₂, —O-cyclopentyl, and —O-cyclohexyl. The term “substituted alkoxy” refers to the group —OR, in which R is a substituted alkyl, as that term is defined above. For example, —OCH₂CF₃ is a substituted alkoxy group.

The term “alcohol” when used without the “substituted” modifier corresponds to an alkane, as defined above, wherein at least one of the hydrogen atoms has been replaced with a hydroxy group. Alcohols have a linear or branched, cyclo, cyclic or acyclic structure. The compounds methanol, ethanol and cyclohexanol are non-limiting examples of alcohols. A “substituted alkane” differs from an alcohol in that it also comprises at least one atom independently selected from the group consisting of N, F, Cl, Br, I, Si, P, and S.

Similarly, the terms “alkenyloxy”, “alkynyloxy”, “aryloxy”, “aralkoxy”, “heteroaryloxy”, “heteroaralkoxy” and “acyloxy”, when used without the “substituted” modifier, refers to groups, defined as —OR, in which R is alkenyl, alkynyl, aryl, aralkyl, heteroaryl, heteroaralkyl and acyl, respectively, as those terms are defined above. When any of the terms alkenyloxy, alkynyloxy, aryloxy, aralkyloxy and acyloxy is modified by “substituted,” it refers to the group —OR, in which R is substituted alkenyl, alkynyl, aryl, aralkyl, heteroaryl, heteroaralkyl and acyl, respectively.
The term “alkylamino” when used without the “substituted” modifier refers to the group —NHR, in which R is an alkyl, as that term is defined above. Non-limiting examples of alkylamino groups include: —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —NH-cyclopentyl, and —NH-cyclohexyl. The term “substituted alkylamino” refers to the group —NHR, in which R is a substituted alkyl, as that term is defined above. For example, —NHCH₂CF₃ is a substituted alkylamino group.

The term “dialkylamino” when used without the “substituted” modifier refers to the group —NRR′, in which R and R′ can be the same or different alkyl groups, or R and R′ can be taken together to represent an alkanediyl having two or more saturated carbon atoms, at least two of which are attached to the nitrogen atom. Non-limiting examples of dialkylamino groups include: —NHC(CH₃)₃, —N(CH₃)CH₂CH₃, —N(CH₂CH₃)₂, N-pyrrolidinyl, and N-piperidinyl. The term “substituted dialkylamino” refers to the group —NRR′, in which R and R′ can be the same or different substituted alkyl groups, one of R or R′ is an alkyl and the other is a substituted alkyl, or R and R′ can be taken together to represent a substituted alkanediyl with two or more saturated carbon atoms, at least two of which are attached to the nitrogen atom.

The terms “alkoxyamino”, “alkenylamino”, “alkynylamino”, “arylamino”, “aralkylamino”, “heteroarylamino”, “heteroaralkylamino”, and “alkylsulfonylamino” when used without the “substituted” modifier, refers to groups, defined as —NHR, in which R is alkoxy, alkenyl, alkynyl, aryl, aralkyl, heteroaryl, heteroaralkyl and alkylsulfonyl, respectively, as those terms are defined above. A non-limiting example of an arylamino group is —NHC₆H₅. When any of the terms alkoxyamino, alkenylamino, alkynylamino, arylamino, aralkylamino, heteroarylamino, heteroaralkylamino and alkylsulfonylamino is modified by “substituted,” it refers to the group —NHR, in which R is substituted alkoxy, alkenyl, alkynyl, aryl, aralkyl, heteroaryl, heteroaralkyl and alkylsulfonyl, respectively.

The term “amido” (acylamino), when used without the “substituted” modifier, refers to the group —NHR, in which R is acyl, as that term is defined above. A non-limiting example of an acylamino group is —NHC(O)CH₃. When the term amido is used with the “substituted” modifier, it refers to groups, defined as —NHR, in which R is substituted acyl, as that term is defined above. The groups —NHC(O)OCH₃ and —NHC(O)NHCH₃ are non-limiting examples of substituted amido groups.

The term “alkylimino” when used without the “substituted” modifier refers to the group=NR, wherein the alkylimino group is attached with one σ-bond and one π-bond, in which R is an alkyl, as that term is defined above. Non-limiting examples of alkylimino groups include: =NCH₃, =NCH₂CH₃ and =N-cyclohexyl. The term “substituted alkylimino” refers to the group=NR, wherein the alkylimino group is attached with one σ-bond and one π-bond, in which R is a substituted alkyl, as that term is defined above. For example, =NCH₂CF₃ is a substituted alkylimino group.

Similarly, the terms “alkenylimino”, “alkynylimino”, “arylimino”, “aralkylimino”, “heteroarylimino”, “heteroaralkylimino” and “acylimino”, when used without the “substituted” modifier, refers to groups, defined as =NR, wherein the alkylimino group is attached with one σ-bond and one π-bond, in which R is alkenyl, alkynyl, aryl, aralkyl, heteroaryl, heteroaralkyl and acyl, respectively, as those terms are defined above. When any of the terms alkenylimino, alkynylimino, arylimino, aralkylimino and acylimino is modified by “substituted,” it refers to the group=NR, wherein the alkylimino group is attached with one σ-bond and one π-bond, in which R is substituted alkenyl, alkynyl, aryl, aralkyl, heteroaryl, heteroaralkyl and acyl, respectively.

The term “fluoroalkyl” when used without the “substituted” modifier refers to an alkyl, as that term is defined above, in which one or more fluorines have been substituted for hydrogens. The groups, —CH₂F, —CF₂H, —CF₃, and —CH₂CF₃ are non-limiting examples of fluoroalkyl groups. The term “substituted fluoroalkyl” refers to a non-aromatic monovalent group with a saturated carbon atom as the point of attachment, a linear or branched, cyclo, cyclic or acyclic structure, at least one fluorine atom, no carbon-carbon double or triple bonds, and at least one atom independently selected from the group consisting of N, O, Cl, Br, I, Si, P, and S. The following group is a non-limiting example of a substituted fluoroalkyl: —CFHOH.

The term “alkylphosphate” when used without the “substituted” modifier refers to the group —OP(O)(OH)(OR), in which R is an alkyl, as that term is defined above. Non-limiting examples of alkylphosphate groups include: —OP(O)(OH)(OMe) and —OP(O)(OH)(OEt). The term “substituted alkylphosphate” refers to the group —OP(O)(OH)(OR), in which R is a substituted alkyl, as that term is defined above.

The term “dialkylphosphate” when used without the “substituted” modifier refers to the group —OP(O)(OR)(OR′), in which R and R′ can be the same or different alkyl groups, or R and R′ can be taken together to represent an alkanediyl having two or more saturated carbon atoms, at least two of which are attached via the oxygen atoms to the phosphorus atom. Non-limiting examples of dialkylphosphate groups include: —OP(O)(OMe)₂, —OP(O)(OEt)(OMe) and —OP(O)(OEt)₂. The term “substituted dialkylphosphate” refers to the group —OP(O)(OR)(OR′), in which R and R′ can be the same or different substituted alkyl groups, one of R or R′ is an alkyl and the other is a substituted alkyl, or R and R′ can be taken together to represent a substituted alkanediyl with two or more saturated carbon atoms, at least two of which are attached via the oxygen atoms to the phosphorous.

The term “alkylthio” when used without the “substituted” modifier refers to the group —SR, in which R is an alkyl, as that term is defined above. Non-limiting examples of alkylthio groups include: —SCH₃, —SCH₂CH₃, —SCH₂CH₂CH₃, —SCH(CH₃)₂, —SCH(CH₂)₂, —S-cyclopentyl, and —S-cyclohexyl. The term “substituted alkylthio” refers to the group —SR, in which R is a substituted alkyl, as that term is defined above. For example, —SCH₂CF₃ is a substituted alkylthio group.

Similarly, the terms “alkenylthio”, “alkynylthio”, “arylthio”, “aralkylthio”, “heteroarylthio”, “heteroaralkylthio”, and “acylthio”, when used without the “substituted” modifier, refers to groups, defined as —SR, in which R is alkenyl, alkynyl, aryl, aralkyl, heteroaryl, heteroaralkyl and acyl, respectively, as those terms are defined above. When any of the terms alkenylthio, alkynylthio, arylthio, aralkylthio, heteroarylthio, heteroaralkylthio, and acylthio is modified by “substituted,” it refers to the group —SR, in which R is substituted alkenyl, alkynyl, aryl, aralkyl, heteroaryl, heteroaralkyl and acyl, respectively.

The term “thioacyl” when used without the “substituted” modifier refers to a monovalent group with a carbon atom of a thiocarbonyl group as the point of attachment, further having a linear or branched, cyclo, cyclic or acyclic structure, further having no additional atoms that are not carbon or hydrogen, beyond the sulfur atom of the carbonyl group. The groups, —CHS, —C(S)CH₃, —C(S)CH₂CH₃, —C(S)CH₂CH₂CH₃, —C(S)CH(CH₃)₂, —C(S)CH(CH₂)₂, —C(S)C₆H₅, —C(S)C₆H₄CH₃, —C(S)C₆H₄CH₂CH₃, —C(S)C₆H₃(CH₃)₂, and —C(S)CH₂C₆H₅, are non-limiting examples of thioacyl groups. The term “thioacyl” therefore encompasses, but is not limited to, groups sometimes referred to as “alkyl thiocarbonyl” and “aryl thiocarbonyl” groups. The term “substituted thioacyl” refers to a radical with a carbon atom as the point of attachment, the carbon atom being part of a thiocarbonyl group, further having a linear or branched, cyclo, cyclic or acyclic structure, further having at least one atom, in addition to the sulfur atom of the carbonyl group, independently selected from the group consisting of N, O, F, Cl, Br, I, Si, P, and S. The groups, —C(S)CH₂CF₃, —C(S)O₂H, —C(S)OCH₃, —C(S)OCH₂CH₃, —C(S)OCH₂CH₂CH₃, —C(S)OC₆H₅, —C(S)OCH(CH₃)₂, —C(S)OCH(CH₂)₂, —C(S)NH₂, and —C(S)NHCH₃, are non-limiting examples of substituted thioacyl groups. The term “substituted thioacyl” encompasses, but is not limited to, “heteroaryl thiocarbonyl” groups.

The term “alkylsulfonyl” when used without the “substituted” modifier refers to the group —S(O)₂R, in which R is an alkyl, as that term is defined above. Non-limiting examples of alkylsulfonyl groups include: —S(O)₂CH₃, —S(O)₂CH₂CH₃, —S(O)₂CH₂CH₂CH₃, —S(O)₂CH(CH₃)₂, —S(O)₂CH(CH₂)₂, —S(O)₂-cyclopentyl, and —S(O)₂-cyclohexyl. The term “substituted alkylsulfonyl” refers to the group —S(O)₂R, in which R is a substituted alkyl, as that term is defined above. For example, —S(O)₂CH₂CF₃ is a substituted alkylsulfonyl group.

Similarly, the terms “alkenylsulfonyl”, “alkynylsulfonyl”, “arylsulfonyl”, “aralkylsulfonyl”, “heteroarylsulfonyl”, and “heteroaralkylsulfonyl” when used without the “substituted” modifier, refers to groups, defined as —S(O)₂R, in which R is alkenyl, alkynyl, aryl, aralkyl, heteroaryl, and heteroaralkyl, respectively, as those terms are defined above. When any of the terms alkenylsulfonyl, alkynylsulfonyl, arylsulfonyl, aralkylsulfonyl, heteroarylsulfonyl, and heteroaralkylsulfonyl is modified by “substituted,” it refers to the group —S(O)₂R, in which R is substituted alkenyl, alkynyl, aryl, aralkyl, heteroaryl and heteroaralkyl, respectively.

The term “alkylsulfinyl” when used without the “substituted” modifier refers to the group —S(O)R, in which R is an alkyl, as that term is defined above. Non-limiting examples of alkylsulfinyl groups include: —S(O)CH₃, —S(O)CH₂CH₃, —S(O)CH₂CH₂CH₃, —S(O)CH(CH₃)₂, —S(O)CH(CH₂)₂, —S(O)— cyclopentyl, and —S(O)— cyclohexyl. The term “substituted alkylsulfinyl” refers to the group —S(O)R, in which R is a substituted alkyl, as that term is defined above. For example, —S(O)CH₂CF₃ is a substituted alkylsulfinyl group.

Similarly, the terms “alkenylsulfinyl”, “alkynylsulfinyl”, “arylsulfinyl”, “aralkylsulfinyl”, “heteroarylsulfinyl”, and “heteroaralkylsulfinyl” when used without the “substituted” modifier, refers to groups, defined as —S(O)R, in which R is alkenyl, alkynyl, aryl, aralkyl, heteroaryl, and heteroaralkyl, respectively, as those terms are defined above. When any of the terms alkenylsulfinyl, alkynylsulfinyl, arylsulfinyl, aralkylsulfinyl, heteroarylsulfinyl, and heteroaralkylsulfinyl is modified by “substituted,” it refers to the group —S(O)R, in which R is substituted alkenyl, alkynyl, aryl, aralkyl, heteroaryl and heteroaralkyl, respectively.

The term “alkylammonium” when used without the “substituted” modifier refers to a group, defined as —NH₂R⁺, —NHRR′⁺, or —NRR′R″⁺, in which R, R′ and R″ are the same or different alkyl groups, or any combination of two of R, R′ and R″ can be taken together to represent an alkanediyl. Non-limiting examples of alkylammonium cation groups include: —NH₂(CH₃)⁺, —NH₂(CH₂CH₃)⁺, —NH₂(CH₂CH₂CH₃)⁺, —NH(CH₃)₂⁺, —NH(CH₂CH₃)₂⁺, —NH(CH₂CH₂CH₃)₂⁺, —N(CH₃)₃⁺, —N(CH₃)(CH₂CH₃)₂⁺, —N(CH₃)₂(CH₂CH₃)⁺, —NH₂C(CH₃)₃⁺, —NH(cyclopentyl)₂⁺, and —NH₂(cyclohexyl)⁺. The term “substituted alkylammonium” refers —NH₂R⁺, —NHRR′⁺, or —NRR′R″⁺, in which at least one of R, R′ and R″ is a substituted alkyl or two of R, R′ and R″ can be taken together to represent a substituted alkanediyl. When more than one of R, R′ and R″ is a substituted alkyl, they can be the same of different. Any of R, R′ and R″ that are not either substituted alkyl or substituted alkanediyl, can be either alkyl, either the same or different, or can be taken together to represent a alkanediyl with two or more carbon atoms, at least two of which are attached to the nitrogen atom shown in the formula.

The term “alkylsulfonium” when used without the “substituted” modifier refers to the group —SRR′⁺, in which R and R′ can be the same or different alkyl groups, or R and R′ can be taken together to represent an alkanediyl. Non-limiting examples of alkylsulfonium groups include: —SH(CH₃)⁺, —SH(CH₂CH₃)⁺, —SH(CH₂CH₂CH₃)⁺, —S(CH₃)₂⁺, —S(CH₂CH₃)₂⁺, —S(CH₂CH₂CH₃)₂⁺, —SH(cyclopentyl)⁺, and —SH(cyclohexyl)⁺. The term “substituted alkylsulfonium” refers to the group —SRR′⁺, in which R and R′ can be the same or different substituted alkyl groups, one of R or R′ is an alkyl and the other is a substituted alkyl, or R and R′ can be taken together to represent a substituted alkanediyl. For example, —SH(CH₂CF₃)⁺ is a substituted alkylsulfonium group.

The term “alkylsilyl” when used without the “substituted” modifier refers to a monovalent group, defined as —SiH₂R, —SiHRR′, or —SiRR′R″, in which R, R′ and R″ can be the same or different alkyl groups, or any combination of two of R, R′ and R″ can be taken together to represent an alkanediyl. The groups, —SiH₂CH₃, —SiH(CH₃)₂, —Si(CH₃)₃ and —Si(CH₃)₂C(CH₃)₃, are non-limiting examples of unsubstituted alkylsilyl groups. The term “substituted alkylsilyl” refers to —SiH₂R, —SiHRR′, or —SiRR′R″, in which at least one of R, R′ and R″ is a substituted alkyl or two of R, R′ and R″ can be taken together to represent a substituted alkanediyl. When more than one of R, R′ and R″ is a substituted alkyl, they can be the same of different. Any of R, R′ and R″ that are not either substituted alkyl or substituted alkanediyl, can be either alkyl, either the same or different, or can be taken together to represent a alkanediyl with two or more saturated carbon atoms, at least two of which are attached to the silicon atom.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

A compound having a formula that is represented with a dashed bond is intended to include the formulae optionally having zero, one or more double bonds. Thus, for example, the structure

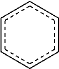

includes the structures

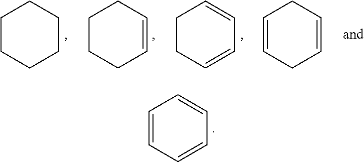

As will be understood by a person of skill in the art, no one such ring atom forms part of more than one double bond.

Any undefined valency on an atom of a structure shown in this application implicitly represents a hydrogen atom bonded to the atom.

As used herein, a “chiral auxiliary” refers to a removable chiral group that is capable of influencing the stereoselectivity of a reaction. Persons of skill in the art are familiar with such compounds, and many are commercially available.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use, 2002.

As used herein, “predominantly one enantiomer” means that a compound contains at least about 85% of one enantiomer, or more preferably at least about 90% of one enantiomer, or even more preferably at least about 95% of one enantiomer, or most preferably at least about 99% of one enantiomer. Similarly, the phrase “substantially free from other optical isomers” means that the composition contains at most about 15% of another enantiomer or diastereomer, more preferably at most about 10% of another enantiomer or diastereomer, even more preferably at most about 5% of another enantiomer or diastereomer, and most preferably at most about 1% of another enantiomer or diastereomer.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerisation, that is, the number of repeat units linked together. When the value for “n” is left undefined, it simply designates repetition of the formula within the brackets as well as the polymeric nature of the material. The concept of a repeat unit applies equally to where the connectivity between the repeat units extends three dimensionally, such as in metal organic frameworks, cross-linked polymers, thermosetting polymers, etc.

The term “saturated” when referring to an atom means that the atom is connected to other atoms only by means of single bonds.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers.

The invention contemplates that for any stereocenter or axis of chirality for which stereochemistry has not been defined, that stereocenter or axis of chirality can be present in its R form, S form, or as a mixture of the R and S forms, including racemic and non-racemic mixtures.

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydropyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃), benzyloxycarbonyl, p-methoxybenzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethyloxycarbonyl groups (such as benzyloxycarbonyl and p-nitrobenzyloxycarbonyl), tert-butoxycarbonyl groups (—C(O)OC(CH₃)₃), and the like. Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethyloxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β-iodoethoxycarbonyl).

“Effective amount,” “therapeutically effective amount” or “pharmaceutically effective amount” means that amount which, when administered to a subject or patient for treating a disease, is sufficient to effect such treatment for the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

As used herein, the term “water soluble” means that the compound dissolves in water at least to the extent of 0.010 mole/liter or is classified as soluble according to literature precedence.

Other abbreviations used herein are as follows: DMSO, dimethyl sulfoxide; NO, nitric oxide; iNOS, inducible nitric oxide synthase; COX-2, cyclooxygenase-2; NGF, nerve growth factor; IBMX, isobutylmethylxanthine; FBS, fetal bovine serum; GPDH, glycerol 3-phosphate dehydrogenase; RXR, retinoid X receptor; TGF-β, transforming growth factor-β; IFNγ or IFN-γ, interferon-γ; LPS, bacterial endotoxic lipopolysaccharide; TNFα or TNF-α, tumor necrosis factor-α; IL-1β, interleukin-1β; GAPDH, glyceraldehyde-3-phosphate dehydrogenase; MTT, 3-[4,5-dimethylthiazol-2-yl]-2,5-diphenyltetrazolium bromide; TCA, trichloroacetic acid; HO-1, inducible heme oxygenase.

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### II. SIX1

Six1 belongs to the Six family of homeobox genes (Six1-6) encoding transcription factors that play vital roles in the development of many organs (Kawakami et al., 2000). Six1-6 share a DNA binding homeodomain (HD) and a Six domain (SD) responsible for co-activator binding (Kawakami et al., 2000). In particular, Six1 plays a role in cell growth, cell survival and cell migration during normal cell development. Six1 plays a critical role in the onset and progression of a significant proportion of breast and other cancers, but has never before been clinically targeted. The Six1 homeobox gene encodes a transcription factor that is crucial for the development of many organs but is down-regulated after organ development is complete. Its expression is low or undetectable in normal adult breast tissue but the gene is over-expressed in 50% of primary breast tumors and 90% of metastatic lesions. Examination of public microarray databases containing more than 535 breast cancer samples demonstrates that Six1 levels correlate significantly with shortened time to relapse, shortened time to metastasis, and decreased overall survival. In addition, Six1 overexpression correlates with adverse outcomes in numerous other cancers, including ovarian, hepatocellular carcinoma, and rhabdomyosarcoma. Using mouse models of mammary cancer, it was recently demonstrated that over-expression of Six1 results in enhanced proliferation, transformation, increased tumor volume, and metastasis. Importantly, RNA interference against Six1 decreases cancer cell proliferation and metastases in several different cancer models.

Six1 was shown to bind tightly to the MEF3 motif (TCAGGTT) (Spitz et al., 1998). This sequence is different from the TAAT core sequence bound by the canonical HD, likely due to the fact that the HD in Six1 differs from the “classic” HD at two highly conserved residues contacting DNA. The Six type HD is believed to confer a unique DNA binding specificity to the Six family members that differs from the TAAT core in the classic HD. However, the consensus Six1 recognition sequence remains unknown. A limited number of potential Six1 targets are identified (Kawakami et al., 1996; Spitz et al., 1998; Ando et al., 2005) and, indeed, none of them contain the TAAT core. Interestingly, these targets do not share an obvious consensus sequence, possibly due to the limited number of sequences analyzed. Recently, an ideal Six1 DNA binding sequence (TGATAC) was identified using combined bioinformatic and biologic approaches (Noyes et al., 2008). The Six1 target most relevant to breast tumorigenesis is the cyclin A1 promoter (Coletta et al., 2004). The transcriptional up-regulation of cyclin A1 by Six1 leads to an increase in proliferation in mammary carcinoma cells and Six1 mediated cell cycle progression is dependent on cyclin A1 (Coletta et al., 2004). In addition to the HD, the Six family members contain a conserved and novel Six-domain (SD) (Oliver et al., 1995). The SD contributes to DNA binding as well as to protein interaction with cofactors (Kawakami et al., 2000; Oliver et al., 1995).

Six1 does not have an intrinsic activation or repression domain and requires the Eya coactivator proteins to activate transcription. The Eya proteins utilize their intrinsic phosphatase activity to switch the Six1 transcriptional complex from a repressor to an activator complex. The Six1-Eya interaction is essential for proliferation during embryonic development, and both Six1 and EYA2 have been independently implicated in the same types of cancer. Because the Eya co-activator contains a unique protein phosphatase domain whose activity is required to activate Six1, it may serve as a novel anti-cancer drug target. Eya knockout mice phenocopy Six1 knockout mice (Xu et al., 1999). Six1's activity on cellular proliferation was also found to be dependent on Eya (Li et al., 2003). As Six1 contributes to breast tumorigenesis by stimulating cellular proliferation, the interaction between Eya and Six1 may be critical for Six1-mediated tumorigenesis. Indeed, in mammals, SIX2, SIX4, and SIX5 are also able to synergize with EYAs to drive expression from a reporter construct. EYA2, SIX1 and DACH2 synergistically regulate myogenesis in chicken somite cultures.

### III. EYA-2

The transcriptional coactivator Eya belongs to a set of evolutionally conserved genes termed the retinal determination gene network in Drosophila. It collectively encodes a cohort of nuclear transcriptional factors and/or cofactors whose expression is regulated by a conserved hierarchy of transcriptional regulation. The retinal determination gene network comprises twin-of eyeless (toy), eyeless (ey), eye absent (eya), sine oculis (so), and dachshund (dac), which are essential for eye fate specification in metazoans and which control essential cellular functions such as proliferation, differentiation, and cell death during organogenesis.

Genetic studies in Drosophila have shown that mutations of members of the retinal determination gene network lead to failures in eye formation, whereas their ectopic expression leads to formation of additional eyes. Eya is thought to function as a transcriptional coactivator with no specific DNA-binding activity. The collective evidence gathered in Drosophila suggests that Eya and so together constitute a functional transcription factor, with Eya providing the activation domain and so contributing the DNA-binding moiety.

Eya family members are defined by a conserved ˜275-amino-acid carboxyl-terminal motif, referred to as the Eya domain (ED), which has been shown to bind two other retinal determination members, so and dac. In addition, the retinal determination gene network functions in a variety of other developmental contexts. Human homologues, EYA1-4, are strikingly similar in their eya domain as well as their NH2 termini, with the exception of a small tyrosine-rich region, named the Eya domain 2.

Eyes absent homolog 2 (EYA2) is a protein that in humans is encoded by the EYA2 gene. This gene encodes a member of the eyes absent (EYA) family of proteins. The encoded protein may be post-translationally modified and may play a role in eye development. A similar protein in mice can act as a transcriptional activator. Five transcript variants encoding three distinct isoforms have been identified for this gene. In addition to playing a role in normal development, EYA2 has been suggested to play an important role in human cancer. Previous data show that EYA2 is expressed at low levels in normal human ovary. The transcription and expression of EYA2 are, however, significantly up-regulated in human ovarian cancer. Moreover, EYA2 is significantly increased in late-stage ovarian cancer, where high expression is significantly associated with poor outcome. These results support a novel role of EYA2 in human ovarian cancer.

One report showed that the eya2 gene is amplified in some ovarian cancer specimens, and thus DNA amplification might partly provide an explanation for the observed EYA2 overexpression in ovarian cancer. However, studies in Drosophila have showed that eyeless (ey) induces expression of eya and so, suggesting that ey is able to directly or indirectly activate eya expression. Data also indicate that human PAXs, the mammalian homologues of ey, are overexpressed or translocated in cancer and may play a role in tumorigenesis. As such, upstream transcriptional regulatory factors involved in EYA2 activation may contribute to EYA2 overexpression in human ovarian cancer. The fact that EYA2 protein is strongly expressed in the nuclei of ovarian cancer cells also indicates that EYA2 might function as a nuclear protein (e.g., transcription factor) in ovarian cancer.

### IV. EYA2 PHOSPHATASE INHIBITORS

Compounds for use with the present invention may be obtained commercially, such as from SigmaAldrich (Milwaukee, Wis.); Chemical Diversity Laboratories (San Diego, Calif.); and ChemBridge Corp. (San Diego, Calif.). Compounds may also be ordered from companies that prepare customized organic compounds (e.g., AsisChem, Inc., SynChem, Inc.), or prepared using synthetic organic techniques. See U.S. Patent Publication 2009/0163545, which is hereby incorporated by reference.

Synthetic techniques to prepare compounds of the present invention as well as basic derivatives thereof are well-known to those of skill in the art. For example, Smith and March, 2001 discuss a wide variety of synthetic transformations, reaction conditions, and possible pitfalls relating thereto. Methods discussed therein may be adapted to prepare compounds of the present invention from commercially available starting materials.

Solvent choices for preparing compounds of the present invention will be known to one of ordinary skill in the art. Solvent choices may depend, for example, on which one(s) will facilitate the solubilizing of all the reagents or, for example, which one(s) will best facilitate the desired reaction (particularly when the mechanism of the reaction is known). Solvents may include, for example, polar solvents and non-polar solvents. Solvents choices include, but are not limited to, tetrahydrofuran, dimethylformamide, dimethylsulfoxide, dioxane, methanol, ethanol, hexane, methylene chloride and acetonitrile. More than one solvent may be chosen for any particular reaction or purification procedure. Water may also be admixed into any solvent choice. Further, water, such as distilled water, may constitute the reaction medium instead of a solvent.

Persons of ordinary skill in the art will be familiar with methods of purifying compounds of the present invention. One of ordinary skill in the art will understand that compounds of the present invention can generally be purified at any step, including the purification of intermediates as well as purification of the final products. In preferred embodiments, purification is performed via silica gel column chromatography or HPLC.

In view of the above definitions, other chemical terms used throughout this application can be easily understood by those of skill in the art. Terms may be used alone or in any combination thereof

Compounds of the present invention may contain one or more asymmetric centers and thus can occur as racemates and racemic mixtures, single enantiomers, diastereomeric mixtures and individual diastereomers. In certain embodiments, a single diastereomer is present. All possible stereoisomers of the compounds of the present invention are contemplated as being within the scope of the present invention. However, in certain aspects, particular diastereomers are contemplated. The chiral centers of the compounds of the present invention can have the S- or the R-configuration, as defined by the IUPAC 1974

Recommendations. In certain aspects, certain compounds of the present invention may comprise S- or R-configurations at particular carbon centers.

Certain biological activity data is available for some of the compounds of the present invention. For example, assay results for MLS000544460-01 are provided at the world-wide-web at:

ncbi.nlm.nih.gov/sites/entrez?db=pccompound&term=MLS000544460 last visited Jan. 6, 2011, the contents of which are incorporated herein by reference.

### V. DETECTION METHODS

In some embodiments, it may prove useful to assess the expression of Eya2 and/or Six1 in a cell from a subject having or suspected of having cancer. In others, miRNAs will be detected. Thus, it is within the general scope of the present invention to provide methods for the detection of proteins and nucleic acids (e.g., mRNAs and miRNAs). Any method of detection known to one of skill in the art falls within the general scope of the present invention.

In particular, nucleic acids can used be as probes or primers for embodiments involving nucleic acid hybridization. As such, they may be used to assess miRNA expression. Commerically available systems, such as Qiagen's miScript System™ are available for detection of miRNAs. Various aspects of nucleic acid detection as discussed below.

A. Protein Detection

1. Immunodetection Methods

As discussed, in some embodiments, the present invention concerns immunodetection methods for binding, purifying, removing, quantifying and/or otherwise detecting biological components such as antigenic regions on polypeptides and peptides. The immunodetection methods of the present invention can be used to identify antigenic regions of a peptide, polypeptide, or protein that has therapeutic implications, particularly in reducing the immunogenicity or antigenicity of the peptide, polypeptide, or protein in a target subject.

Immunodetection methods include enzyme linked immunosorbent assay (ELISA), radioimmunoassay (RIA), immunoradiometric assay, fluoroimmunoassay, chemiluminescent assay, bioluminescent assay, and Western blot, though several others are well known to those of ordinary skill. The steps of various useful immunodetection methods have been described in the scientific literature, such as, e.g., Doolittle and Ben-Zeev (1999); Gulbis et al. (1993); De Jager et al. (1993); and Nakamura et al. (1987), each incorporated herein by reference.

In general, the immunobinding methods include obtaining a sample suspected of containing a protein, polypeptide and/or peptide, and contacting the sample with a first antibody, monoclonal or polyclonal, in accordance with the present invention, as the case may be, under conditions effective to allow the formation of immunocomplexes.

These methods include methods for purifying a protein, polypeptide and/or peptide from organelle, cell, tissue or organism's samples. In these instances, the antibody removes the antigenic protein, polypeptide and/or peptide component from a sample. The antibody will preferably be linked to a solid support, such as in the form of a column matrix, and the sample suspected of containing the protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen or antigenic domain, and contact the sample with an antibody against the antigen or antigenic domain, and then detect and quantify the amount of immune complexes formed under the specific conditions.

In terms of antigen detection, the biological sample analyzed may be any sample that is suspected of containing an antigen or antigenic domain, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes).

The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

2. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, antibodies are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and/or then contacted with antibodies. After binding and/or washing to remove non-specifically bound immune complexes, the bound anti-antibodies are detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

3. Immunohistochemistry

The antibodies of the present invention may also be used in conjunction with both fresh-frozen and/or formalin-fixed, paraffin-embedded tissue blocks prepared for study by immunohistochemistry (IHC). For example, immunohistochemistry may be utilized to characterize Six1 or to evaluate the amount Six1 in a cell. The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Immunohistochemistry or IHC refers to the process of localizing proteins in cells of a tissue section exploiting the principle of antibodies binding specifically to antigens in biological tissues. It takes its name from the roots “immuno,” in reference to antibodies used in the procedure, and “histo,” meaning tissue. Immunohistochemical staining is widely used in the diagnosis and treatment of cancer. Specific molecular markers are characteristic of particular cancer types.

Visualizing an antibody-antigen interaction can be accomplished in a number of ways. In the most common instance, an antibody is conjugated to an enzyme, such as peroxidase, that can catalyse a color-producing reaction. Alternatively, the antibody can also be tagged to a fluorophore, such as FITC, rhodamine, Texas Red, Alexa Fluor, or DyLight Fluor. The latter method is of great use in confocal laser scanning microscopy, which is highly sensitive and can also be used to visualize interactions between multiple proteins.

Briefly, frozen-sections may be prepared by rehydrating 50 mg of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

There are two strategies used for the immmunohistochemical detection of antigens in tissue, the direct method and the indirect method. In both cases, the tissue is treated to rupture the membranes, usually by using a kind of detergent called Triton X-100.

The direct method is a one-step staining method, and involves a labeled antibody (e.g. FITC conjugated antiserum) reacting directly with the antigen in tissue sections. This technique utilizes only one antibody and the procedure is therefore simple and rapid. However, it can suffer problems with sensitivity due to little signal amplification and is in less common use than indirect methods.

The indirect method involves an unlabeled primary antibody (first layer) which reacts with tissue antigen, and a labeled secondary antibody (second layer) which reacts with the primary antibody. The secondary antibody must be against the IgG of the animal species in which the primary antibody has been raised. This method is more sensitive due to signal amplification through several secondary antibody reactions with different antigenic sites on the primary antibody. The second layer antibody can be labeled with a fluorescent dye or an enzyme.

In a common procedure, a biotinylated secondary antibody is coupled with streptavidin-horseradish peroxidase. This is reacted with 3,3′-Diaminobenzidine (DAB) to produce a brown staining wherever primary and secondary antibodies are attached in a process known as DAB staining. The reaction can be enhanced using nickel, producing a deep purple/gray staining

The indirect method, aside from its greater sensitivity, also has the advantage that only a relatively small number of standard conjugated (labeled) secondary antibodies needs to be generated. For example, a labeled secondary antibody raised against rabbit IgG, which can be purchased “off the shelf,” is useful with any primary antibody raised in rabbit. With the direct method, it would be necessary to make custom labeled antibodies against every antigen of interest.

4. Protein Arrays

Protein array technology is discussed in detail in Pandey and Mann (2000) and MacBeath and Schreiber (2000), each of which is herein specifically incorporated by reference. These arrays, typically contain thousands of different proteins or antibodies spotted onto glass slides or immobilized in tiny wells, allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds. In certain embodiments such technology can be used to quantitate a number of proteins in a sample, such as Six1.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery. The earliest and best-known protein chip is the ProteinChip by Ciphergen Biosystems

Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum). The system also utilizes biomarker pattern software that automates pattern recognition-based statistical analysis methods to correlate protein expression patterns from clinical samples with disease phenotypes.

B. Nucleic Acid Detection

Nucleic acids can used be as probes or primers for embodiments involving nucleic acid hybridization. As such, they may be used to assess mRNA expression for Eya2 and/or Six1. Various aspects of nucleic acid detection as discussed below.

1. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In particular embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772 and U.S. Patent Publication 2008/0009439. The relevant portions of these and other references identified in this section of the Specification are incorporated herein by reference.

2. In Situ Hybridization

In situ hybridization (ISH) is a type of hybridization that uses a labeled complementary DNA or RNA strand (i.e., probe) to localize a specific DNA or RNA sequence in a portion or section of tissue (in situ), or, if the tissue is small enough (e.g. plant seeds, Drosophila embryos), in the entire tissue (whole mount ISH). This is distinct from immunohistochemistry, which localizes proteins in tissue sections. Fluorescent DNA ISH (FISH) can, for example, be used in medical diagnostics to assess chromosomal integrity. RNA ISH (hybridization histochemistry) is used to measure and localize mRNAs and other transcripts within tissue sections or whole mounts.

For hybridization histochemistry, sample cells and tissues are usually treated to fix the target transcripts in place and to increase access of the probe. As noted above, the probe is either a labeled complementary DNA or, now most commonly, a complementary RNA (riboprobe). The probe hybridizes to the target sequence at elevated temperature, and then the excess probe is washed away (after prior hydrolysis using RNase in the case of unhybridized, excess RNA probe). Solution parameters such as temperature, salt and/or detergent concentration can be manipulated to remove any non-identical interactions (i.e., only exact sequence matches will remain bound). Then, the probe that was labeled with either radio-, fluorescent- or antigen-labeled bases (e.g., digoxigenin) is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

3. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to any sequence corresponding to a nucleic acid sequence are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al. (1988), each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Reverse transcription (RT) of RNA to cDNA followed by quantitative PCR (RT-PCR) can be used to determine the relative concentrations of specific mRNA species isolated from a cell, such as a Six1 or Eya-encoding transcript. By determining that the concentration of a specific mRNA species varies, it is shown that the gene encoding the specific mRNA species is differentially expressed. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

A second condition for an RT-PCR experiment is to determine the relative abundances of a particular mRNA species. Typically, relative concentrations of the amplifiable cDNAs are normalized to some independent standard. The goal of an RT-PCR experiment is to determine the abundance of a particular mRNA species relative to the average abundance of all mRNA species in the sample.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

RT-PCR can be performed as a relative quantitative RT-PCR with an internal standard in which the internal standard is an amplifiable cDNA fragment that is larger than the target cDNA fragment and in which the abundance of the mRNA encoding the internal standard is roughly 5-100-fold higher than the mRNA encoding the target. This assay measures relative abundance, not absolute abundance of the respective mRNA species.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Various nucleic acid detection methods known in the art are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Chip Technologies and Nucleic Acid Arrays

The present invention may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon. Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

### VI. METHODS OF THERAPY

In some embodiments, the invention provides compositions and methods for the treatment of cancer. In one embodiment, the invention provides a method of treating cancer comprising administering to a patient an effective amount of an EYA2 inhibitor. This treatment may be further combined with additional cancer treatments. One of skill in the art will be aware of many treatments that may be combined with the methods of the present invention, some but not all of which are described below.

In general, the cancers will be characterized by overexpression of Six1 and/or EYA2. Thus, it is contemplated that a wide variety of tumors may be treated using Six1 miRNAs, including cancers of the brain, lung, liver, spleen, kidney, lymph node, pancreas, small intestine, blood cells, colon, stomach, breast, endometrium, prostate, testicle, ovary, skin, head and neck, esophagus, bone marrow, blood or other tissue.

In many contexts, it is not necessary that the tumor cell be killed or induced to undergo normal cell death or “apoptosis.” Rather, to accomplish a meaningful treatment, all that is required is that the tumor growth be slowed to some degree. It may be that the tumor growth is completely blocked, however, or that some tumor regression is achieved. Clinical terminology such as “remission” and “reduction of tumor” burden also are contemplated given their normal usage.

A. Formulations and Routes for Administration to Patients

In some embodiments, the invention provides a method of treating cancer comprising providing to a patient an effective amount of a compound as provided herein. Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions. Of particular interest is direct intratumoral administration, perfusion of a tumor, or administration local or regional to a tumor, for example, in the local or regional vasculature or lymphatic system, or in a resected tumor bed (e.g., post-operative catheter). For practically any tumor, systemic delivery also is contemplated. This will prove especially important for attacking microscopic or metastatic cancer.

The active compounds may also be administered as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

“Treatment” and “treating” refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

A “disease” can be any pathological condition of a body part, an organ, or a system resulting from any cause, such as infection, genetic defect, and/or environmental stress.

“Prevention” and “preventing” are used according to their ordinary and plain meaning to mean “acting before” or such an act. In the context of a particular disease, those terms refer to administration or application of an agent, drug, or remedy to a subject or performance of a procedure or modality on a subject for the purpose of blocking the onset of a disease or health-related condition.

The subject can be a subject who is known or suspected of being free of a particular disease or health-related condition at the time the relevant preventive agent is administered. The subject, for example, can be a subject with no known disease or health-related condition (i.e., a healthy subject).

In additional embodiments of the invention, methods include identifying a patient in need of treatment. A patient may be identified, for example, based on taking a patient history or based on findings on clinical examination.

B. Cancer Combination Treatments

In some embodiments, the method further comprises treating a patient with cancer with a conventional cancer treatment. One goal of current cancer research is to find ways to improve the efficacy of chemo- and radiotherapy, such as by combining traditional therapies with other anti-cancer treatments. In the context of the present invention, it is contemplated that this treatment could be, but is not limited to, chemotherapeutic, radiation, a polypeptide inducer of apoptosis or other therapeutic intervention. It also is conceivable that more than one administration of the treatment will be desired.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with gen silencing therapy discussed above. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p9′7), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). 4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Gene Therapy

In yet another embodiment, the secondary treatment is a gene therapy in which a therapeutic polynucleotide is administered before, after, or at the same time as a Six1 or Eya inhibitor is administered. Delivery of a Six1 or Eya inhibitor in conjunction with a vector encoding one of the following gene products may have a combined anti-hyperproliferative effect on target tissues. A variety of proteins are encompassed within the invention, some of which are described below.

a. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA or siRNA directed to a particular inducer of cellular proliferation is used to prevent expression of the inducer of cellular proliferation.

The proteins FMS and ErbA are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

b. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, mda-7, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/Six1 or Eya2, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

c. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

d. miRNAs

The inventors have previously disclosed compositions and methods relating to Six1 miRNAs for the treatment of cancer. These compositions, which can be used advantageously in combination with the compositions of the present invention, are described in PCT US2010/043354, the entire contents of which are hereby incorporated by reference.

6. Other Agents

It is contemplated that other agents may be used with the present invention. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon α, β, and γ; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

E. Dosage

An miRNA can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of antagomir (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of antagomir per kg of bodyweight. The unit dose, for example, can be administered by injection (e.g., intravenous or intramuscular, intrathecally, intratumorally or directly into an organ), inhalation, or a topical application.

Delivery of an miRNA directly to an organ can be at a dosage on the order of about 0.00001 mg to about 3 mg per organ, or particularly about 0.0001-0.001 mg per organ, about 0.03-3.0 mg per organ, about 0.1-3.0 mg per organ or about 0.3-3.0 mg per organ.

Significant modulation of target gene expression may be achieved using nanomolar/submicromolar or picomolar/subnamomolar concentrations of the oligonucleotide, and it is typical to use the lowest concentration possible to achieve the desired resultant increased synthesis, e.g., oligonucleotide concentrations in the 1-100 nM range are contemplated; more particularly, the concentration is in the 1-50 nM, 1-25 nM, 1-10 nM, or picomolar range. In particular embodiments, the contacting step is implemented by contacting the cell with a composition consisting essentially of the oligonucleotide.

In one embodiment, the unit dose is administered once a day, e.g., or less frequently less than or at about every 2, 4, 8 or 30 days. In another embodiment, the unit dose is not administered with a frequency (e.g., not a regular frequency). For example, the unit dose may be administered a single time. Because oligonucleotide agent can persist for several days after administering, in many instances, it is possible to administer the composition with a frequency of less than once per day, or, for some instances, only once for the entire therapeutic regimen.

An miRNA featured in the invention can be administered in a single dose or in multiple doses. Where the administration of the miRNA is by infusion, the infusion can be a single sustained dose or can be delivered by multiple infusions. Injection of the miRNA can be directly into the tissue at or near the site of interest. Multiple injections of can be made into the tissue at or near the site.

In a particular dosage regimen, the miRNA is injected at or near a disease site once a day for seven days, for example, into a tumor, a tumor bed, or tumor vasculature. Where a dosage regimen comprises multiple administrations, it is understood that the effective amount of the miRNA administered to the subject can include the total amount of miRNA administered over the entire dosage regimen. One skilled in the art will appreciate that the exact individual dosages may be adjusted somewhat depending on a variety of factors, including the specific antagomir being administered, the time of administration, the route of administration, the nature of the formulation, the rate of excretion, the particular disorder being treated, the severity of the disorder, the pharmacodynamics of the oligonucleotide agent, and the age, sex, weight, and general health of the patient. Wide variations in the necessary dosage level are to be expected in view of the differing efficiencies of the various routes of administration. Variations in these dosage levels can be adjusted using standard empirical routines of optimization, which are well-known in the art. The precise therapeutically effective dosage levels and patterns can be determined by the attending physician in consideration of the above-identified factors.

In one embodiment, a subject is administered an initial dose, and one or more maintenance doses of an miRNA. The maintenance dose or doses are generally lower than the initial dose, e.g., one-half less of the initial dose. The maintenance doses are generally administered no more than once every 5, 10, or 30 days. Further, the treatment regimen may last for a period of time which will vary depending upon the nature of the particular disease, its severity and the overall condition of the patient. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

Certain factors may influence the dosage required to effectively treat a subject, including but not limited to the severity of the disease or disorder, previous treatments, the general health and/or age of the subject, and other diseases present. It will also be appreciated that the effective dosage of the antagomir used for treatment may increase or decrease over the course of a particular treatment. Changes in dosage may result and become apparent from the results of diagnostic assays. For example, the subject can be monitored after administering an antagomir composition. Based on information from the monitoring, an additional amount of the antagomir composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on IC₅₀'s found to be effective in in vitro and in vivo animal models.

### VII. EXAMPLES

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

### Small Molecule Screen

Materials and Method

Protein Expression and Purification.

Human Eya2 ED (residue 253-538) that contains the Eya phosphatase activity was sub-cloned into the pGEX-6P1 (GE Healthcare) vector using the BamHI and XhoI site and confirmed by DNA sequencing. Plasmids containing these constructs were transformed into E. coli strain XA90. Cells were grown until OD₆₀₀ reached 0.8-1.0 and protein expression was induced at 20° C. with 0.2 mM IPTG for 20 hours. Cell pellets were lysed by sonication in lysis buffer (50 mM Tris, pH 7.5, 250 mM NaCl, 5% glycerol, 1 mM DTT) containing protease inhibitors pepstatin A, leupeptin, and PMSF. Lysates were cleared via centrifugation (2×45 minutes at 18,000×g). GST-Eya2 ED protein in the supernatant were purified on glutathione-Sepharose 4B resin (GE Healthcare). Eya2 protein were cleaved off the glutathione resin with PreScission protease, concentrated, and further purified on a Superdex 200 size exclusion column (GE Healthcare). Purified protein was aliquoted and stored at −80° C.

OMFP-Based Eya Phosphatase Assay.

The activity of ED was measured in 50 μL reactions in black, 96-well, half-volume microtiter plates (Greiner Bio-one) with OMFP (3-O-methylfluorescein phosphate, Sigma-Aldrich) as the substrate. Upon dephosphorylation, OMFP is converted to a fluorescent product OMF. The assay was performed in 50 mM MES, pH 6.5, 50 mM NaCl, 5 μM MgCl₂, 0.05% BSA, 1 mM DTT and contained 50 nM Eya2 ED and 100 μM OMFP. Reactions were started by the addition of OMFP and were continued for 1 hour at room temperature and terminated by the addition of 75 mM EDTA. Fluorescence intensity was measured at 485/515 nm excitation/emission on a Fluoromax-3® plate reader (Horiba Jobin Yvon). Kinetic experiments were performed using 50 nM Eya2 ED and increasing concentrations of the OMFP substrate (up to 650 μM). Fluorescence intensities were recorded at ten-minute increments for one hour. Initial velocities were plotted against OMFP concentration and were fitted with the Michaelis-Menton equation using KaleidaGraph to generate kinetic parameters.

Miniaturized Eya Phosphatase Assay for HTS.

The OMFP-based Eya2 ED phosphatase assay were miniaturized and optimized in 1536-well black assay plates (Greiner Bio-One). 1.5 μL/well of 200 nM Eya2 ED was incubated with or without 23 nL compound or DMSO control for 10 minutes followed by an addition of 1.5 μL/well of 50 μM OMFP and incubated for 30 minutes. The resulting fluorescence intensity was measured on a Viewlux® plate reader (PerkinElmer) with an excitation wavelength of 485 nm and emission of 525 nm. Since there were no Eya2 inhibitors available during the assay development, the inventors used EDTA as a positive control to access the assay quality.

Compound Library and Instruments for Liquid Handling.

The LOPAC library (Library of Pharmacologically Active Compounds, Sigma-Aldrich) consisting of 1,280 compounds was used for the assay validation. The collection of 331,609 compounds for the primary screen was provided by the NIH's Molecular Library Initiative (pubchem.ncbi.nlm.nih.gov/). All compounds were dissolved in DMSO as 10 mM stock solutions. All compounds were serially diluted at 1:5 ratio in DMSO in 384-well plates for 4 concentrations using a CyBi®-Well dispensing station with a 384-well head (Cybio) and then reformatted into 1536-well plates at 7 μL/well. An automated dispensing station (BioRAPTR® FRD, Beckman Coulter) was used to dispense reagents into 1536-well plates at volumes of 1-3 μL/well. Compounds were transferred to 1536-well assay plates in 23 nl/well using an automated pin-tool station (Kalypsys®).

HTS Data Analysis.

The primary screening data was analyzed as previously described (Southhall et al., 2009). IC₅₀ values were calculated from the fluorescence signal intensity using the Prism® software (Graphpad Software, Inc.). The Z′ factor index of assay quality control (Zhang et al., 1999) was defined as 1-(3*SSR/R), where SSR is the summation of the standard deviation of positive inhibition controls and negative inhibition controls and R is the mean of the positive controls minus the mean of negative controls. All values were expressed as mean+/−SD.

pH2AX-Based Eya Phosphatase Assay.

pH2AX phosphatase assays (50 μL) were carried out in transparent, 96-well, half-area microplates (Greiner Bio-One). The assay was performed at pH 6.0 in 50 mM MES, 50 mM NaCl, 5 μM MgCl₂, 0.05% BSA, 1 mM DTT and contained 3.9 μM ED and 50 μM pH2AX peptide (KATQASQEpY, Abgent). Reactions were allowed to proceed for 40 minutes at 37° C. and terminated by the addition of 100 μL malachite green solution (Millipore). The free phosphate released from dephosphorylation forms a green complex with malachite green and molybdate that can be monitored using absorbance at 620 nm. After a 20-minute incubation at room temperature, the absorbance was measured using a Spectramax PLUS® 384 plate reader (Molecular Devices).

To evaluate the effect of a compound on ED's phosphatase activity, various concentrations of the compound were incubated with ED for 10 minutes prior to the addition of pH2AX that starts the reaction. Dose response curves were generated and IC₅₀ values calculated using Origin Pro 8.0.

Phosphatase assays of PTP1B, PPM1A, and Scp1.

The phosphatase assay of PTP1B was conducted in 30 mM Tris, pH 7.5, 75 mM NaCl, 1 mM EDTA, 0.033% BSA, 1 mM DTT using 20 nM PTP1B (R&D Systems) and 50 μM OMFP as the substrate. To evaluate the effect of a compound on PTP 1 B's phosphatase activity, various concentrations of the compound were incubated with PTP1B for 10 minutes prior to the addition of OMFP to start the reaction. Reactions were in 50 μL volumes and were set up in black, 96-well, half area microplates and proceeded for 30 minutes followed by the addition of Na₃VO₄ to terminate the reaction. Fluorescence intensity was measured at 485/515 nm excitation/emission using a Fluoromax-3 plate reader (Horiba Jobin Yvon).

The phosphatase assay of PPM1A (ProSpec) was conducted in 50 mM Tris, pH 7.5, 0.1 mM EDTA, 0.5 mM DTT, 10 mM MgCl₂ using 0.5 ng/μL PPM1A and 100 μM OMFP. The effect of compounds on PPM1A's phosphatase activity was evaluated similarly as for PTP1B, except that NaF was used to terminate the reaction.

The phosphatase assay of Scp1 (a gift from Dr. Jessie Zhang, UT-Austin) was conducted in 50 mM MES, pH 5.5, 25 mM NaCl, 20 mM MgCl₂ using 2.5 ng/μL Scp1 and 100 μM OMFP. The effect of compounds on Scp1's phosphatase activity was evaluated similarly as for PTP1B, except that NaF was used to terminate the reaction.

Luciferase Assay.

MCF7 cells were plated in 24-well plates at 25,000 cells/well. 24 hours later, cells were transfected with 300 ng/well pGL3-6xMEF3 (a promoter-luciferase construct in which the promoter contains 6 well characterized Six1 MEF3 binding sites (Spitz et al., 1998)), 70 ng/well renilla luciferase, 100 ng/well pcDNA3.1 Six1, and 200 ng/well of pcDNA3.1-FLAG-Eya2 using FuGENE 6 (Roche Applied Science) transfection reagent. The following day the media was changed and 24 hours later lysates were prepared and analyzed with the Dual Luciferase Kit (Promega) on a Modulus Microplate (Turner Biosystems). An Eya2 phosphatase-dead mutant (D274N) was also used in place of wild type Eya2 to evaluate the effect of Eya2's phosphatase activity on transcription. To assay the effect of Eya2 phosphatase inhibitors on transcriptional activity, compound was added 24 hours after transfection and incubated for 48 hours before lysates were prepared and analyzed for luciferase activity as described above. All results were normalized to renilla activity to account for transfection efficiency and transcriptional activity was normalized to Eya2 protein expression in the Eya2 D274N rescue experiment. The inhibitory effect of the compounds was normalized to vehicle-treated control and statistical significance was determined by performing a one-way ANOVA with Dunnett's post-test on the natural log transformed normalized transcriptional activity.

### Results

Known Phosphatase Inhibitors do not Significantly Inhibit Eya2's Phosphatase Activity.

The inventors chose the human Eya2 ED, which contains the Tyr phosphatase activity, for these studies. The ED has comparable phosphatase activity as full length Eya (Li et al., 2003; Rayapureddi et al., 2003; Tootle et al., 2003) and is more stable and thus easier to purify in the quantities necessary for HTS. They expressed the human Eya2 ED in E. coli as a GST-fusion protein. The protein was purified on a glutathione resin, cleaved off the GST tag, and further purified using a Superdex-200 column.

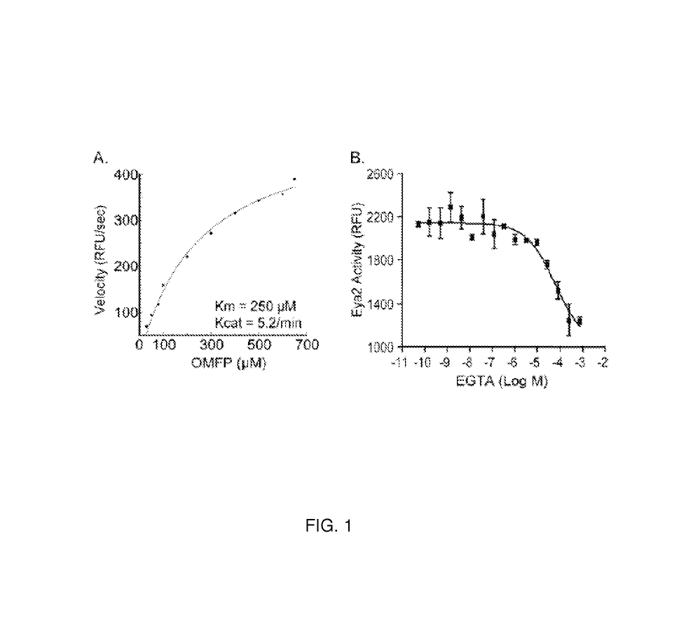

A phosphatase assay was then developed using purified human Eya2 ED and the small molecule OMFP (3-O-methylfluorescein phosphate) as a substrate. OMFP is dephosphorylated to yield a fluorescent product (3-O-methylfluorescein) that can be detected at 485/515 nm excitation/emission wavelengths. The inventors demonstrated that purified ED is active with a kcat=5.2/min and Km=250 μM (FIG. 1A). Like other HAD family members, EDs are Mg²⁺-dependent phosphatases (Li et al., 2003; Rayapureddi et al., 2003; Tootle et al., 2003). The inventors have shown that EGTA inhibits the phosphatase activity with an IC₅₀ of 65 μM (FIG. 1B).

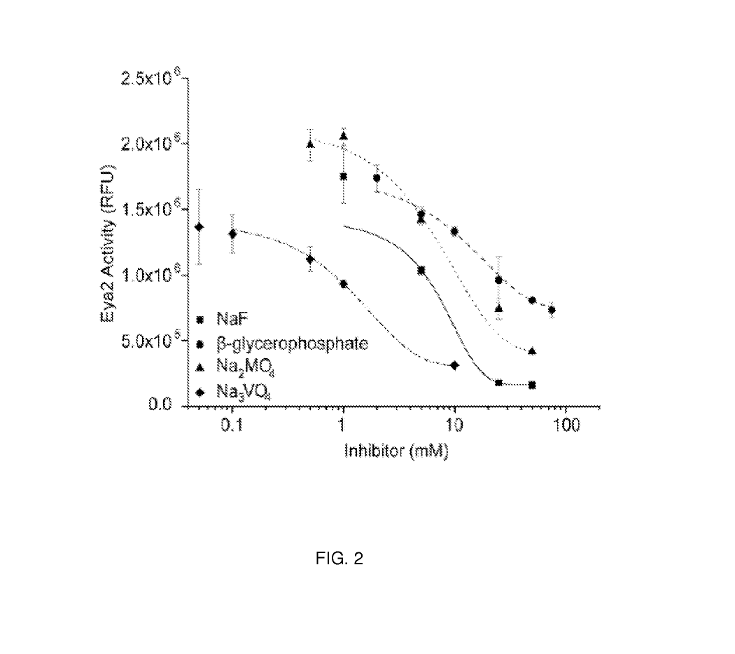

The inventors reasoned that existing phosphatase inhibitors would likely not inhibit the phosphatase activity of ED, because the ED of Eya is an unique HAD phosphatase (Jung et al., 2010) and there are no known specific HAD phosphatase inhibitors. Thus, they tested nine common phosphatase inhibitors against the Eya2 ED including okadaic acid (inhibitor of Ser/Thr phosphatase PP2A), L-phenylalanine (intestinal alkaline phosphatase inhibitor), cyclosporine A (calcineurin inhibitor through binding to cyclophilin), (1,10)-phenanthroline, phenylarsine oxide (protein tyrosine phosphatase inhibitor), NaF (phospho-serine or threonine inhibitor), Na₃VO₄ (protein phosphotyrosyl phosphatase inhibitor), Na₂MnO₄ (protein phosphotyrosyl phosphatase inhibitor), and β-glycerophosphate. Five inhibitors (okadaic acid, L-phenylalanine, cyclosporine A, 1,10-phenanthroline, and phenylarsine oxide) were inactive when tested against ED's phosphatase activity at concentrations that completely inhibit their cognitive phosphatases (data not shown). Four other inhibitors only inhibited ED's phosphatase activity at very high concentrations, with IC₅₀s of 11.3 mM for Na₂MnO₄, 8.2 mM for beta-glycerophosphate, 6.6 mM for NaF, and 1.8 mM for Na3VO4 (FIG. 2). These data suggest that known phosphatase inhibitors do not significantly inhibit Eya's phosphatase activity. This, in combination with the functional significance of Eya's phosphatase activity in tumorigenesis, metastasis, and DNA repair, prompted us to carry out a large scale HTS to identify potent and specific inhibitors of the Eya phosphatase activity.

The OMFP-based phosphatase assay is suitable for HTS.

The OMFP-based phosphatase assay was optimized and miniaturized in a 1536-well format for adaptation to HTS. The Eya2 enzyme concentration was first titrated in the assay using OMFP as a substrate. The enzyme activity linearly increased with the increase of enzyme concentration until at least 1.25 μM of Eya2 (data not shown). To minimize the consumption of enzyme in later large scale primary screening, 100 nM Eya2 concentration was selected that produced sufficient assay signal. Additionally, the lower protein concentrations used in the compound screen could increase the assay sensitivity to potential inhibitors. The signal-to-basal ratio at this enzyme concentration was ˜8 fold, which reproduced in multiple experiments. After substrate concentration titration experiments, 25 μM OMFP concentration was selected for further experiments that yielded sufficient assay signal window.

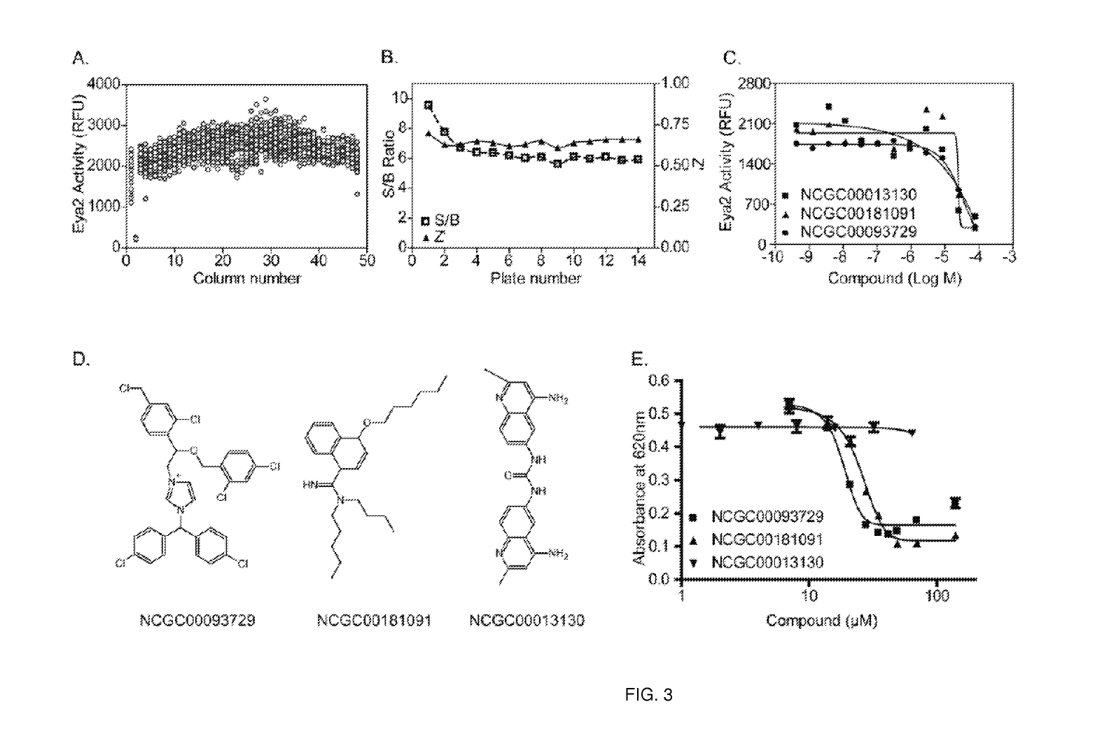

The OMFP-based Eya2 phosphatase assay was miniaturized into a 3 μl/well assay in the 1536-well plate format for HTS. A DMSO plate was first used to assess the assay performance. The signal-to-basal ratio was 7.75 and the Z′ factor (Zhang et al., 1999) was 0.7 (FIG. 3A), indicating a robust assay that is suitable for HTS. The inventors then carried out a pilot screen using the LOPAC library of 1,280 compounds with pharmacologically known activities (Sigma-Aldrich) as well as a 2800 compound approved drug set (NIH Chemical Genomics Center (NCGC) collection). Each compound was screened in a seven-concentration titration ranging from 0.122 to 76.6 μM as previously described (Inglese et al., 2006). The average signal-to-basal ratio was 7 fold and the Z factor was 0.7 (FIG. 3B), similar to what was observed in the DMSO plate test. A total of three compounds were identified as primary hits with a hit selection criteria of IC50<35 μM and maximal inhibition>80%, after eliminating obvious metal chelators (FIGS. 3C-D). The hit rate from this test screen was 0.55%, which is an acceptable rate for HTS.

A Large Scale Primary Screen Identified a Class of Structurally Related Initial Hits.

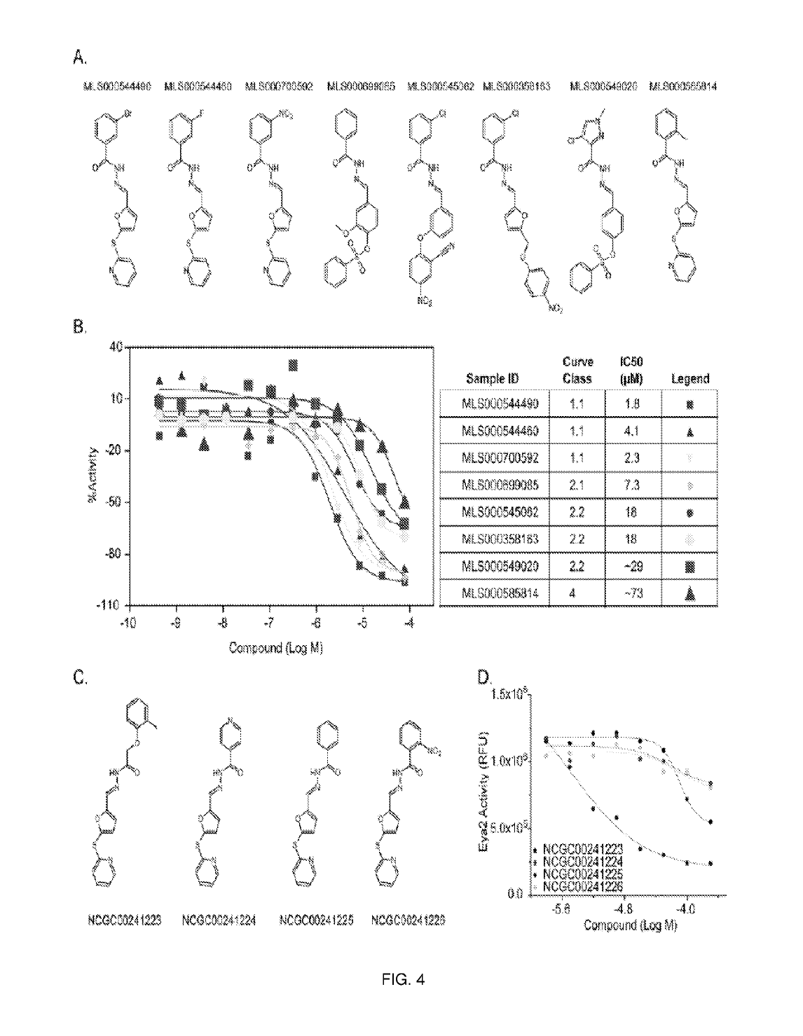

The inventors next carried out a large-scale screen of 329,717 compounds from the MLPCN (Molecular Libraries Probe Centers Network) at four different concentrations ranging from 0.61 to 76.7 μM in a quantitative HTS format (Inglese et al., 2006). Dose responses were assigned to different curve classes to evaluate the results of qHTS (Inglese et al., 2006). Curve classes 1.1 and 2.1 both demonstrate high compound efficacy with either complete (class 1.1) or partial (class 2.1) dose response curves. Compounds in these two curve classes have the highest chances of reproducing and are typically considered the most promising compounds from a primary screen. A few compounds increased the phosphatase activity in the primary screen and may warrant further investigation as possible activators of the Eya2 phosphatase. In this example, the inventors decided to focus on compounds that inhibit the Eya2 phosphatase activity, with the hope that these may be promising leads for anti-cancer drugs. The overall hit rate of the primary screen was low and only 3 inhibitory compounds belonged to class 1.1 and 7 belonged to class 2.1. However, all three class 1.1 compounds, one class 2.1 compound, and four compounds from other curve classes, clearly belong to the same structural class with a N-arylidenebenzohydrazide core (FIG. 4A). The inventors decided to focus on this class of compounds for further characterization.

A 12-point dose response curve was then carried out and validated all eight compounds as inhibitors of ED's phosphatase activity in the OMFP-based phosphatase assay (FIG. 4B). The inventors also carried out the inhibition assay under high Mg²⁺ (30 μM-1.25 mM) to exclude metal chelators, in the absence of DTT to remove nonspecific covalent modifiers, and by adding compounds immediately before plate reading to rule out quenchers that interfere with readout of the assay (data not shown). All eight compounds passed these false positive tests and were thus considered highly promising primary screen hits.

Following this initial analysis, the inventors identified four other commercially available compounds that have the same core structure as this class of initial hits (FIG. 4C). The inventors tested these compounds using the OMFP-based phosphatase assay (FIG. 4D). Compound NCGC00241225 is the most active with an IC₅₀ of 3.45 μM. Compound NCGC00241223 has an IC₅₀ of 79.11 04 while compounds NCGC00241224 and NCGC00241226 are essentially inactive.

The Best Compounds from this Series are Active in a pH2AX-Based Secondary Phosphatase Assay.

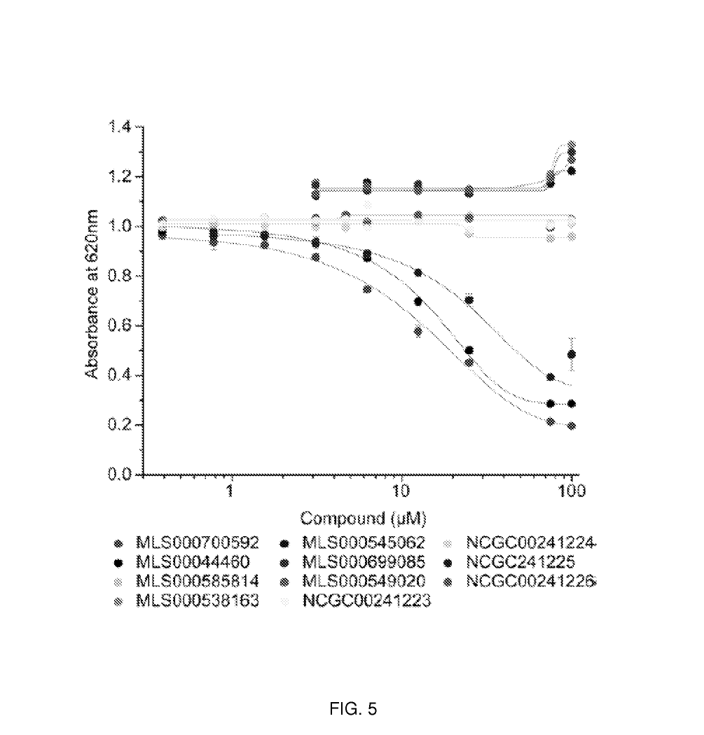

To further confirm that this class of compounds are authentic inhibitors of the Eya2 phosphatase, the inventors developed a malachite green-based secondary phosphatase assay using phosphorylated H2AX peptide (pH2AX), a known physiological substrate of the Eya phosphatase (Krishnan et al., 2009; Cook et al., 2009). In this assay, inorganic phosphate released from the H2AX peptide upon dephosphorylation by ED forms a colored complex with malachite green and molybdate, of which the absorbance can be measured at 620 nm. The inventors demonstrated that two of the class 1.1 compounds (another class 1.1 compound was not tested due to limited availability of the compound) and another structurally highly related compound (NCGC00241225) are active in the pH2AX assay with IC₅₀ in the range of 16.8 to 45.2 μM (FIG. 5). Several other compounds with higher IC50 in the OMFP assay (FIGS. 4B-C) do not significantly inhibit ED's phosphatase activity in the pH2AX assay (FIG. 5), maintaining the rank order of IC₅₀s observed in the primary OMFP assay (FIGS. 4A-D).

In addition, the inventors tested the three hits from the pilot screen and two of the compounds (NCGC00181091 and NCGC00093729) are active in the H2AX-based phosphatase assay with IC₅₀s of 31.98 and 18.25 μM, respectively (FIG. 3F). In the event that the inventors have difficulty optimizing the N-arylidenebenzohydrazide series of compounds, these additional compounds can be further optimized.

The N-Arylidenebenzohydrazide Compounds do not Inhibit Other Cellular Phosphatases.

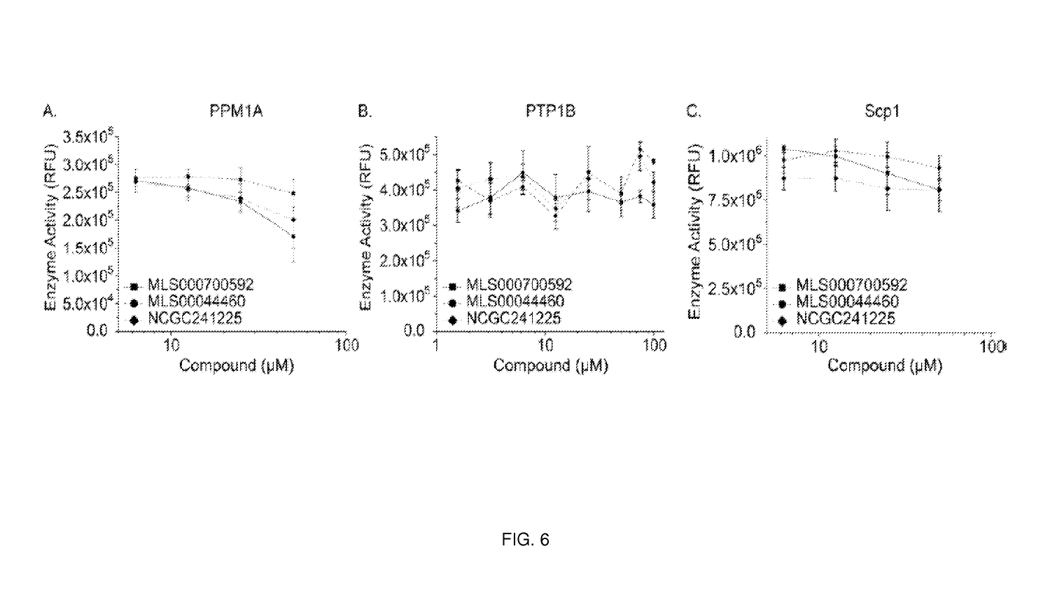

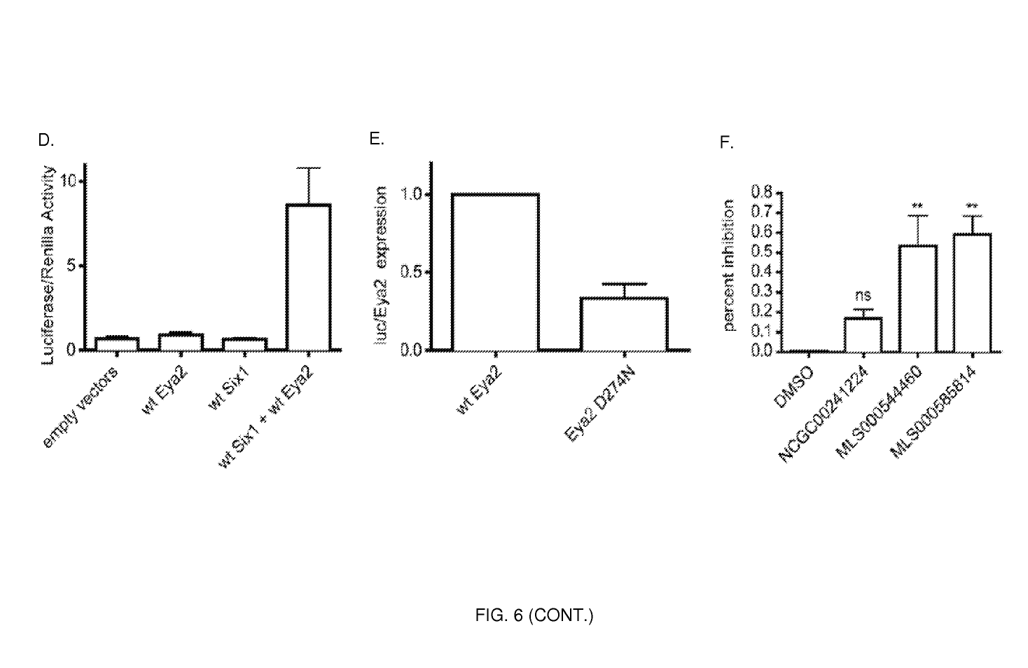

A counter screen was then carried out on the most potent compounds to evaluate their specificity against a number of other cellular phosphatases. The other phosphatases examined include PTP1B, a prototypic protein tyrosine phosphatase that does not require Mg²⁺ for catalytic activity; PPM1A, a Ser/Thr phosphatase that, like Eya2, requires Mg²⁺ for catalytic function (Marley et al., 1996); and Scp1, another HAD family protein phosphatase (Zhang et al., 2010; Ghosh et al., 2008). Scp1 is an Mg²⁺-dependent phosphatase that, like Eya2, uses a catalytic aspartic acid as the nucleophile in the dephosphorylation reaction. However, Scp1 dephosphorylates phospho-Serine, unlike Eya2, the only known HAD member that dephosphorylates phosphor-Tyrosine. The inventors demonstrated that this inhibitor series do not ignificantly inhibit the above phosphatases tested (FIGS. 6A-C), demonstrating specificity against the Eya2 phosphatase.

The N-Arylidenebenzohyrdrazide Compounds Inhibit Six1-Mediated Transcription.

The inventors then tested the effect of several of the identified inhibitors in cell culture using a Six1-mediated transcription assay. Six1, in collaboration with Eya2, activates a reporter containing MEF3 DNA binding sites (a well-known Six1 target sequence (Spitz et al., 1998)) upstream of a luciferase reporter gene (FIG. 6D). The Eya2 phosphatase-dead mutant (D274N) is compromised in its ability to serve as a coactivator for Six1 (FIG. 6E). Eya2 phosphatase inhibitors (MLS00044460 and MLS000585814) diminish Six1/Eya2 mediated transcription from the MEF-luciferase reporter. However, NCGC00241224, which belongs to the same structural class of the lead series but does not significantly inhibit the Eya2 phosphatase in the OMFP assay (FIG. 4D), does not significantly affect Six1/Eya2-mediated transcription from the MEF3-luciferase reporter (FIG. 6F). These data demonstrate that this class of inhibitors has the ability to penetrate the cell to elicit an effect on Six1/Eya2-mediated transcription.

### Discussion

The Eya family of proteins are clearly essential for Six1-mediated breast tumorigenesis and metastasis (Farabaugh et al., 2011). Knockdown of Eya2 in MCF7 cells inhibits the ability of Six1 to induce TGF-β signaling, epithelial-mesenchymal transition (EMT), and tumor initiating cell (TIC) characteristics, properties that are associated with Six1-induced tumorigenesis and metastasis (Farabaugh et al., 2011). Since the phosphatase activity of Eya is important for a subset of transcription mediated by Six1, it is possible that Eya's phosphatase activity also influence Six1-mediated breast tumorigenesis and metastasis. Indeed, Hegde and colleagues have shown that Eya's phosphatase activity is important for the transformation, migration, invasion, and metastasis of breast cancer cells (Pandey et al., 2010), although the role of the Eya phosphatase activity, per se, in mediating Six1-induced metastasis remains unknown. The phosphatase activity of Eya also plays an important role in directing cells to the DNA repair instead of apoptosis pathway upon DNA damage (Krishnan et al., 2009; Cook et al., 2009). Therefore, inhibitors of the Eya phosphatase activity can potentially inhibit breast tumorigenesis/metastasis and/or serve as sensitizers for therapeutics that acts by inducing DNA damage. Although many phosphatases have been described as attractive targets for drug discovery (Jailkhani et al., 2011; Pereira et al., 2011), the difficulty in obtaining small molecules that inhibit phosphatases in a selective manner is well known and is mostly due to the high degree of similarity between the catalytic domains of the enzymes. The Eya proteins, as novel HAD family phosphatases that target phosphor-Tyr, offer us a unique opportunity to potentially identify specific Eya phosphatase inhibitors.

To that end, the inventors developed a fluorescent HTS phosphatase assay using the Eya2 ED and small molecule OMFP as a substrate. It is standard practice to use artificial, small molecule substrates for HTS phosphatase assays since these assays are sensitive, robust, and inexpensive compared to assays using phosphorylated peptide or protein substrates (Tierno et al., 2007). The inventors carried out a qHTS of over 300,000 compounds that allowed for the identification of a series of small molecule inhibitors of Eya2 phosphatases. Analogs within this chemotype ranged from no activity to low micromolar IC₅₀s, indicating inherent SAR within the series. For example, the benzohydrazide substitution was tolerated in the meta position but not the ortho position. The furanyl-2-thio-2-pyridine substituent of the N-arylidene functional group provided best activity although other benzylidene substituents seemed to be tolerated.

Furthermore, the inventors carried out an orthogonal malachite green-based phosphatase assay monitoring phosphate release from the H2AX peptide substrate, which confirmed the qHTS results. There is a reduction of IC₅₀ values in the H2AX assay compared to the OMFP assay, which is not surprising given the different assay format and somewhat lower sensitivity of the malachite green assay. The inventors' specificity assays showed that these inhibitors do not significantly inhibit several other cellular phosphatases, including a prototypic protein tyrosine phosphatase PTP1B, an Mg²⁺-dependent Ser/Thr phosphatase PPM1A, and a Ser/Thr HAD family protein phosphatase Scp1. In addition, the inventors have shown that the phosphatase activity of Eya2 is important for Six1-mediated transcriptional activation of the MEF3 promoter using an Eya2 phosphatase-dead mutant. The inventors went on to demonstrate that the series of identified compounds can reduce Six1-mediated transcription in a promoter-luciferase assay, indicating that these compounds can enter the cell and inhibit the phosphatase activity of Eya2. The mechanism of how the inhibition of Eya2's phosphatase activity affects Six1/Eya2-mediated transcription is not yet clear. One possibility is that these compounds bind the active site of the Eya2 phosphatase, causing a conformational change at the Six1/Eya2 binding interface or the Six1/Eya2/DNA interface and consequently affecting Six1/Eya2-mediated transcription. It is also possible that Eya2 dephosphorylates a protein that is involved in the Six1/Eya2 transcriptional complex that consequently affects Six1/Eya2-mediated transcription. It is foreseeable that inhibitors of Eya2's phosphatase activity can potentially inhibit the pro-tumorigenic functions of Six1. The inventors plan to further evaluate whether these compounds inhibit the transformation, migration, and invasion properties of breast cancer cells and whether these compounds inhibit DNA repair mediated by H2AX under DNA damaging conditions. The inventors also plan to carry out structure activity relationship studies of this series and further optimize these compounds to improve potency and specificity.

It is worth noting that Park et al. recently carried out a virtual screening using the crystal structure of the Eya2 ED and identified 7 compounds with IC₅₀ ranging from 4.4 to 66.2 μM in a fluorescent phosphatase assay using small molecule 6,8-difluoro-4-methylumberlliferyl phosphate (DIFMUP) as a substrate (Park et al., 2011). These compounds were proposed to bind to the active site of Eya and chelate the active site Mg²⁺, although the specificity of these compounds against Eya has not been assessed. In general, efforts from the inventors' laboratories and others devoted to identifying potent and specific inhibitors of the Eya phosphatase may one day lead to a new approach in the treatment of cancer.

### Example 2

### Inhibition Mechanism

### Materials and Methods

Protein Expression and Purification.

Eya2 was sub-cloned on a pGEX-6p1 vector (GE Healthcare) and the protein was expressed as a GST-fusion protein from E. coli. The protein was first purified on a glutathione column. GST was then cleaved off using PreScission protease and eluted Eya2 ED was further purified on a Superdex 200 (S200) column (GE Healthcare).

Eya2 Phosphatase Assays.

A fluorogenic Eya2 phosphatase assay was developed using 3-O-methylfluorescein phosphate (OMFP, Sigma-Aldrich) as a substrate. OMFP produces O-methylfluorescein (OMF) upon hydrolysis by Eya2 phosphatase. Assay development and optimization were carried out in black 1536-well plates (Greiner Bio-one). For all experiments, assay plates were shaded from lights and room temperature (˜21° C.) incubation was used. Assay buffer was comprised of 50 mM HEPES (pH 7.5), 50 mM NaCl, 0.05% BSA, 1 mM DTT, and indicated amounts of MgCl₂. Briefly, 2.5 μn/well of 200 nM Eya2 was incubated with or without 23 nl compound, MgCl₂, EDTA, or DMSO control for 10 minutes followed by an addition of 2.5 μL/well of 50 μM OMFP and incubation for 30 minutes. The resulting fluorescence intensity in the assay plates was detected on a Viewlux plate reader (PerkinElmer) with excitation of 485 nm and emission of 525 nm.

Thermal Shift Experiments.

In order to provide evidence for the binding between hydrazides with Eya2, thermal shift experiments were performed to evaluate if Eya2 melting temperature (Tₘ) could be positively shifted in the presence of different concentrations of hydrazides. Compounds were serially diluted in DMSO in 96-well polypropylene plates (Thermo Fisher Scientific). A mixture of 49 μl of Eya2 and SYPRO Orange (5000× stock solution, Invitrogen) were transferred to a 96-well skirted thin-wall PCR plate (Bio-Rad) with a final concentration of 1 μM and 5×, respectively. Compounds prepared in the aforementioned mother plate were subsequently added to the protein-dye mixture, with final concentrations ranging from 0.34 nM to 200 μM (final volume 50 μl). DMSO alone was also transferred within each compound dilution series as a control sample. DMSO concentration was consistent across each dilution series and was maintained at 2%. The plate was sealed with Optical-Quality Sealing Tape (Bio-Rad) and was centrifuged at 1000 rpm for 20 s before heating commenced. Plate heating was performed on an iQ5 real-time PCR detection system (Bio-Rad) from 20 to 95° C. with an increment of 1° C. and a ramping rate of 0.1° C./s. A CCD camera was used to measure SYPRO Orange fluorescence through a filter set corresponding to excitation and emission wavelengths of 490 and 575 nm, respectively. Protein melting temperature was obtained from an EXCEL-based worksheet (provided by Structure Genomics Consortium, ftp.sgc.ox.au.uk/pub/biophysics and GraphPad Prism 4.

UV-Vis Spectra Analysis of Selected Hydrazides.

Compounds were dissolved in acetonitrile at a final concentration of 25 μM with final Mg²⁺ concentration at 0, 0.5, 1, 5, 10, 25, 50, 100 and 200 mM. UV-visible spectra were obtained using an Agilent 8453 UV-Visible Spectrophotometer (Agilent Technologies) in 0.1 cm quartz cuvette at room temperature.

¹NMR Analysis of Selected Hydrazides—Eya2 Kinetic Experiments.

Due to the solubility limitations and relatively high Kₘ values of the OMFP substrate, the inventors developed a second fluorogenic Eya2 phosphatase assay using a different substrate, fluorescein diphosphate (FDP, Anaspec), to measure compounds' effect on enzyme kinetics. All kinetic experiments were performed in 384 black medium binding plates (Greiner Bio-one). Using the FDP substrate, the inventors were able to achieve an FDP stock solution of 100 mM in Eya2 ED buffer, therefore allowing us to test Eya2 kinetic experiments at the appropriate substrate versus compound concentrations to calculate an accurate kₘ value and to generate a Lineweaver-Burke plot for Eya2 ED. Briefly, 2.5 μl of compound (MLS000544460) at 100, 40, 20, 10, 5 or 0 μM was incubated with 2.5 μl of 2 μM Eya2 ED for 10 minutes. Subsequently, 5 μl of FDP substrate at 20, 12, 7.2, 4.3, 2.6, 1.6, 1, or 0 mM was added and plates were incubated for an additional 30 minutes before measured on the Viewlux plate reader with excitation of 485 nm and emission of 525 nm. Kinetic analysis was performed using GraphPad Prism 4 (GraphPad Software, Inc.).

Molecular Docking.

The AutoDock 4.0 program was used for docking the compounds to the crystal structure of ED of Eya2 (pdb code 3HB1). The active site of the protein was defined by a grid of 70×70×70 points with a grid spacing of 0.375 Å centered at Mg²⁺ ion. The Lamarckian Genetic Algorithm (LGA) was applied with 100 runs and the maximum number of energy evaluations was set to 2×10⁶. Results differing by less than 1.5 Å in positional root-mean-square deviation (RMSD) of substrate were clustered and the final binding conformations were represented by the one with the most favorable free energy of binding. The optimal binding complexes were subjected a stepwise energy minimization and MD simulations.

Molecular Dynamics Simulations.

MD simulations were conducted for modeled inhibitors with Eya2 in explicit solvent using the AMBER 9.0 package and the Parm99 force field. The solvated protein systems were subjected to a thorough energy minimization prior to MD simulations by first minimizing the water molecules while holding the solute frozen (1000 steps using the steepest descent algorithm), followed by 5000 steps of conjugate gradient minimization of the whole system to remove close contacts and to relax the system. Bond lengths involving hydrogen were constrained with SHAKE and the time step for all MD simulations was set to 2 fs. A non-bonded cutoff of 10 Å was used, and the non-bonded pair list was updated every 25 time steps. Periodic boundary conditions were applied to simulate a continuous system. The particle mesh Ewald (PME) method was employed to calculate the long-range electrostatic interactions (Darden and Pedersen, 1993). The simulated system was first subjected to a gradual temperature increase from 0 K to 300 K over 100 ps, and then equilibrated for 500 ps at 300 K, followed by production runs of 2 ns length in total. The resulting trajectories were analyzed using the PTRAJ module from the AMBER package.

### Results

HTS Identified a New Chemical Series that Specifically Inhibit Eya's Phosphatase Activity.

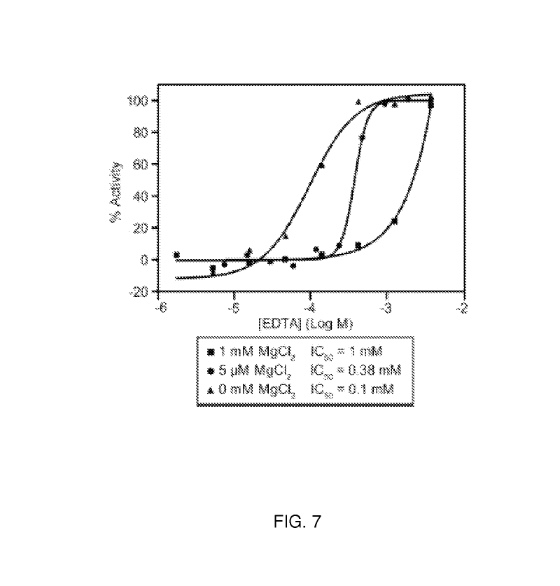

The inventors first developed an OMFP-based phosphatase assay using the Eya2 Eya Domain (ED), which contains similar phosphatase activity as the full-length Eya2. OMFP is dephosphorylated to produce O-methylfluorescein (OMF) whose fluorescence can be detected at 485 nm excitation/525 nm emission wavelengths (Morris et al., 1998). The phosphatase activity of Eya, like other HAD family phosphatases, is Mg²⁺ dependent (Tootle et al., 2003). As expected, the enzymatic activity of the Eya2 ED is inhibited by EDTA and IC₅₀ of EDTA inhibition increases with increasing concentrations of Mg²⁺ (FIG. 7).

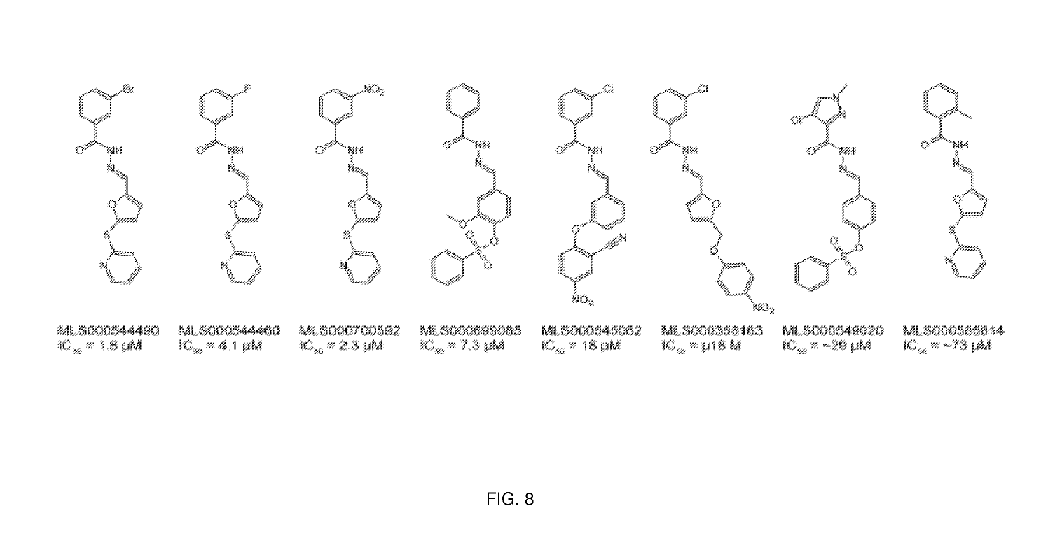

The inventors carried out a quantitative HTS (Southall et al., 2009) of 329,717 compounds from the MLPCN (Molecular Libraries Probe Production Centers Network) library using this assay and identified a class of inhibitors containing an N-arylidenebenzohydrazide core that inhibit ED's phosphatase activity with 1.8-73 μM IC₅₀s (FIG. 8). The inventors confirmed the activity of these compounds through an orthogonal malachite green based phosphatase assay using the H2AX peptide as substrate and demonstrated that this series of compounds do not significantly inhibit other cellular phosphatases including PTP1B, PPM1A, and Scp1. In summary, the inventors' HTS has identified a new chemical series that specifically inhibit the Eya2 phosphatase activity.

Eya2 Inhibitors Stabilize ED in the Absence of Mg²⁺ and Destabilize ED in the Presence of Mg²⁺.

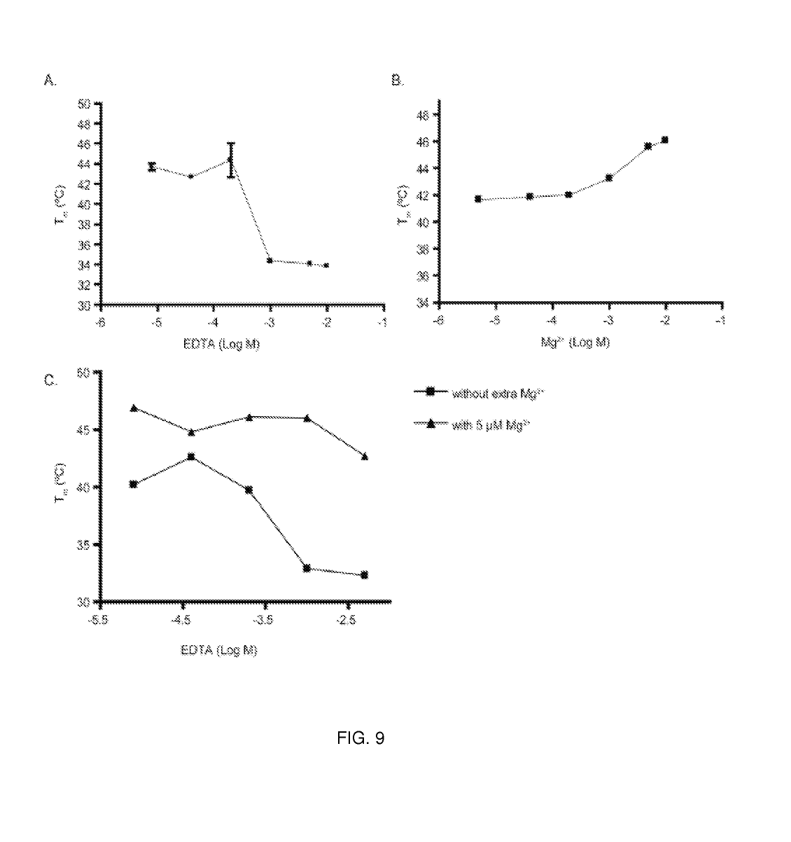

The inventors next carried out protein thermal shift experiments to examine the binding interaction between these compounds and the ED. They first evaluated the effect of Mg²⁺ on the melting temperatures (Tₘ) and protein stability of ED. They found that the addition of EDTA, an Mg²⁺-chelating agent, destabilizes the protein and reduces Tₘ in a dose-dependent manner (FIG. 9A). In contrast, the addition of exogenous Mg²⁺ stabilizes the protein and increases Tₘ (FIG. 9B), indicating that the levels of Mg²⁺ within the Eya2 preparation are below saturation. Moreover, the inventors demonstrate that even 5 mM EDTA is not able to overcome the stabilization effect of 5 μM Mg²⁺, indicating that the enzyme has greater affinity for Mg²⁺ than the chelating agent (FIG. 9C).

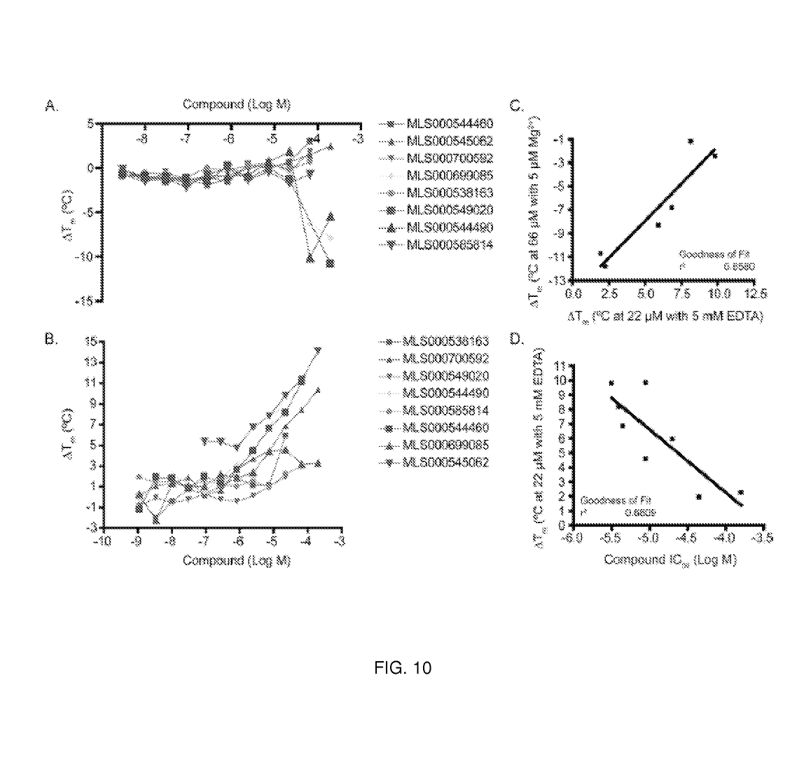

The inventors next examined the ability of Eya2 inhibitors to modulate Tₘ in the presence and absence of 5 μM Mg²⁺. In general, hit compounds tend to destabilize the protein and lead to a reduction of Tₘ (compared to the absence of any compounds) in the presence of Mg²⁺ (FIG. 10A). These compounds tend to stabilize the protein after the endogenous Mg²⁺ is chelated by EDTA (FIG. 10B). Importantly, there is a good correlation between the ability of these compounds to stabilize the protein in the presence of EDTA and their ability to destabilize the protein in the presence of 5 μM Mg²⁺ (FIG. 10C).

It is well known that, within a chemical series, small molecule induced increase in protein melting temperature directly correlates with its affinity towards the protein. Thus, it can be suggested that, those compounds within this chemical series that are able to induce a higher melting temperature in the presence of 5 mM EDTA have higher affinity towards the ED. There is indeed a correlation between the increase in melting temperatures and the IC₅₀ observed in the OMFP-based phosphatase assay (FIG. 10D). Compounds that induce higher Tₘ (therefore likely have greater affinity toward the ED) also show better inhibitory capacity (lower IC₅₀) in the functional assay (FIG. 10D).

Eya2 Inhibitors have a Low Intrinsic Affinity Toward Mg²⁺.

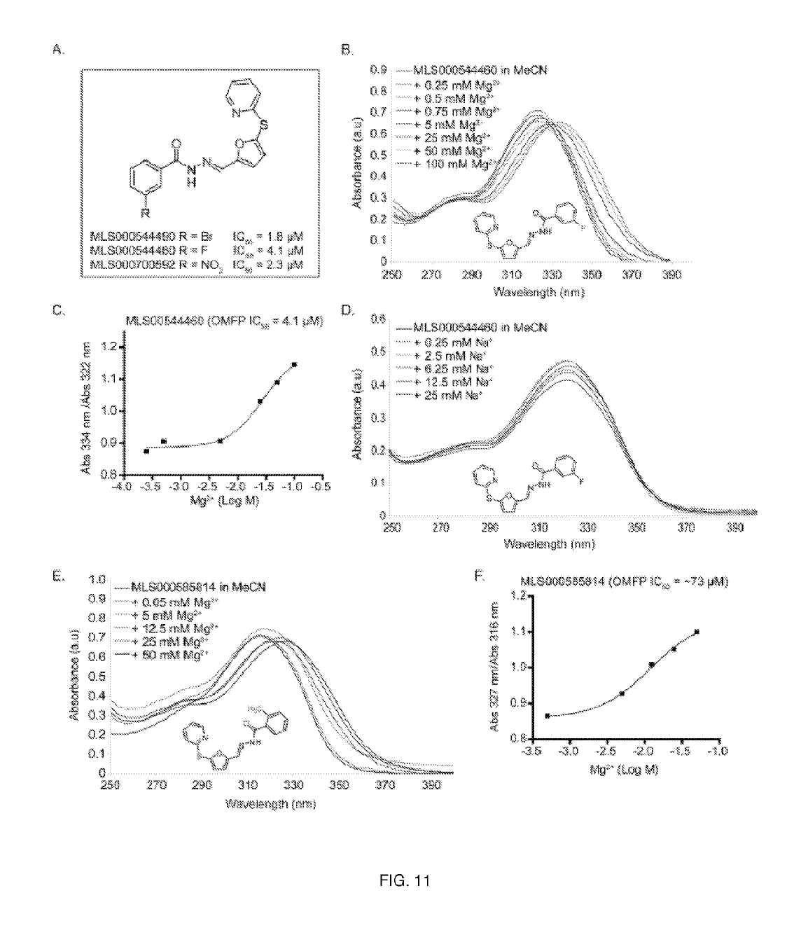

A preliminary structural analysis of the hit compounds, especially the top three active compounds, suggested the presence of a number of heteroatoms (FIG. 11A) that can coordinate the Mg²⁺ ion. A simple way to analyze this possible coordination is to examine the change in the UV spectra of the compound with increasing concentrations of Mg²⁺. Initial Mg²⁺ titrations with a constant level of the hit compound MLS000544460 in aqueous buffers (including the phosphatase assay buffer) displayed no change in the UV spectra of the compound, implying a lack of coordination with the metal in aqueous solution (data not shown).

In order to elicit any possible weak association to Mg²⁺, the inventors moved on to the organic solvent acetonitrile (Hojo et al., 2002). They recorded the UV absorption spectra of a 25 μM solution of MLS000544460 in the presence of various amounts of magnesium perchlorate in acetonitrile. The solutions were prepared such that the absorbance was within the 0-1 range. They observed a dose-dependent shift in the maximum absorbance wavelength (λₘₐₓ), from 322 nm (0 mM Mg²⁺) to 334 nm (100 mM Mg²⁺) (FIGS. 11B, 11C). The inventors conducted a similar study with Na where the compound concentration was kept at 12.5 μM and the sodium perchlorate concentration ranged from 0 to 25 mM. They did not observe any shift in the λₘₐₓ with Na⁺ (FIG. 10D). Altogether, these results suggest that the inhibitor has a very weak intrinsic affinity for Mg²⁺. In addition, this affinity also seems to be specific for Mg²⁺ as little shift in the λₘₐₓ was observed in similar studies with Na⁺.

Subsequently, the inventors evaluated the low activity inhibitor MLS000585814 to test if the weak affinity towards Mg²⁺ had any relationship with the potency of these compounds. A 25 μM solution of MLS000585814 in acetonitrile was mixed with increasing concentrations of Mg²⁺ (0-50 mM). A similar degree of λₘₐₓ shift was observed, from the initial 316 nm when no Mg²⁺ was present to 327 nm when 50 mM Mg²⁺ was present (FIG. 11E). Although this compound has very low inhibitory activity in the enzymatic assay, it did exhibit binding towards Mg²⁺ with a similar weak affinity as the more potent compound MLS000544460 (FIG. 11F). This suggests that the binding affinity of these compounds to the ED, but not their intrinsic affinity towards Mg²⁺, is the dominant factor in dictating their inhibitory activity.

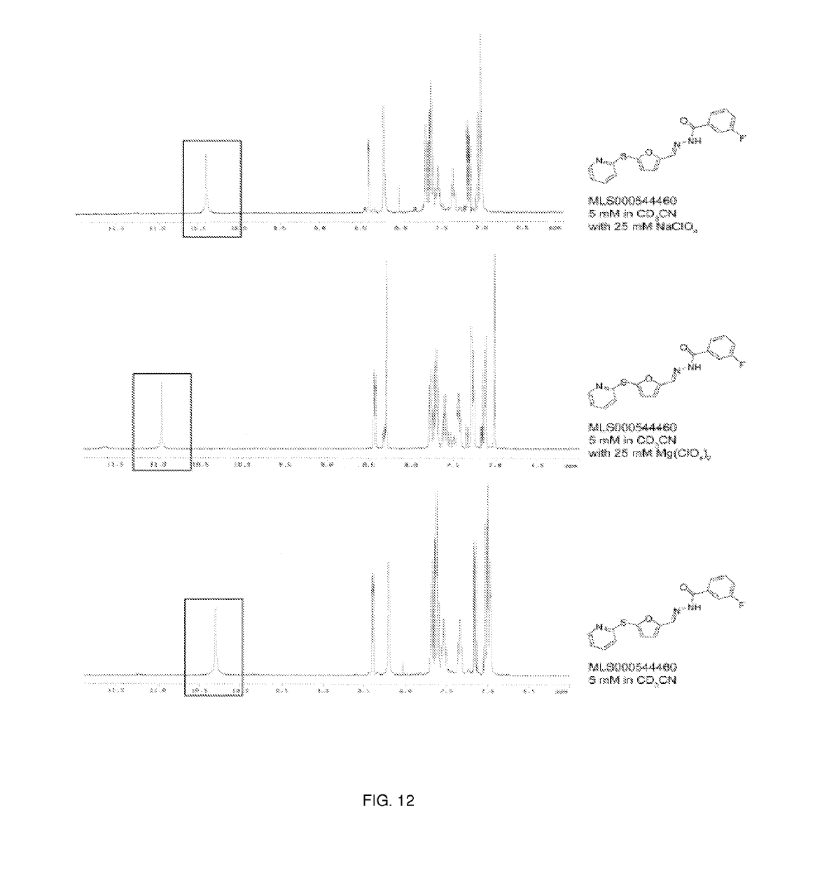

The inventors further carried out ¹H NMR spectroscopy studies to evaluate the interaction between these compounds and Mg²⁺. The ¹H NMR spectrum of a 5 mM solution of MLS000544460 in CD₃CN in the presence of 25 mM magnesium or sodium perchlorate show that most of the chemical shifts remained unchanged with Mg²⁺ although the peak shapes were altered (FIG. 12). The hydrazide proton exhibited the most significant chemical shift change of ˜0.6 ppm implying that this functional group may be the one weakly associating with Mg²⁺. This association could result in a deshielding of the hydrazide NH upon coordination between the carbonyl oxygen and Mg²⁺. The same experiment with Na⁺ hardly showed any change in the ¹H NMR spectrum. The splitting patterns were virtually identical to the spectrum with no metal ion. The hydrazide NH only shifted about 0.1 ppm. This reiterated the inventors' findings from the UV studies that these compounds do not have a general propensity to form a complex with metals and that their ability to complex with Mg²⁺, albeit weak, is likely selective.

Eya2 Inhibitors are Reversible and Mixed-Mode Inhibitors.

In order to further characterize the mechanism of action of these compounds, the inventors carried out classical enzymatic analyses. One possible mechanism of action for these compounds is that the hydrazide functional group provides a reactive chemical moiety for irreversible binding to the enzyme. To evaluate this possibility, the inventors conducted a traditional irreversibility experiment where the compound and enzyme were pre-incubated at a much higher concentration (100×) followed by dilution to the concentration typically used for the enzymatic assay. Compounds binding in an irreversible manner will inactivate the enzyme and exhibit increased potency on dilution and the enzyme will not be able to regain the activity it demonstrated at the lower concentration. In these experiments it is also important to measure the enzymatic activity at different time points after dilution to allow for equilibration.

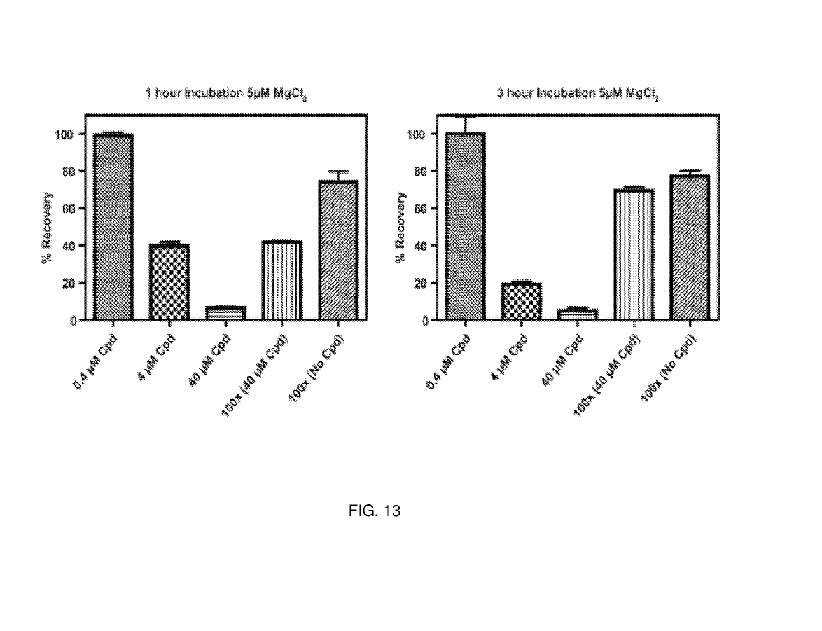

The inventors first examined the Eya2 phosphatase inhibition capacity of MLS000544460 at 0.4, 4.0 and 40 μM concentrations using 1 nM ED. A clear dose dependent inhibition with an IC₅₀ in the range of 4 μM was observed. The inventors then incubated 40 μM compound and 100 nM enzyme for 10 min, diluted the sample by 100-fold, incubated the sample for additional 1 or 3 hr, then added the substrate and measured enzyme activity in 20 minutes (100×(40 μM) column in FIG. 13). The enzyme slowly regains its activity after the dilution although it requires about 3 hours for almost complete functional recovery (compared to the 100×(no compound) control column which is the enzyme activity after bringing the enzyme concentration to 100 nM then dilute 100-fold in the absence of any compound). This indicates that these inhibitors have a slow dissociation rate from the enzyme but are not irreversible binders. The reduced activity (˜80%) within the 100× control column (no compound) is probably caused by the partial precipitation and potential denaturation of enzyme observed at high compound concentrations.

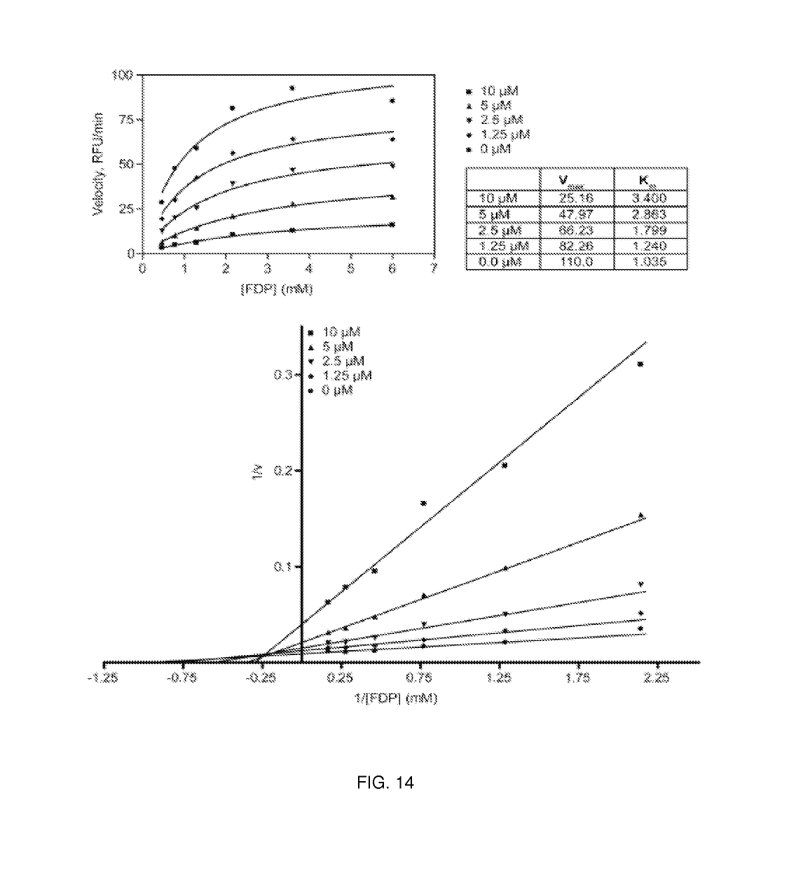

The inventors further carried out kinetic competition experiments using the more soluble FDP as a substrate (FIG. 14). The classical Lineweaver-Burke analysis of the inhibitor suggests a mixed mode behavior, between competitive and non-competitive inhibition, indicating that the enzyme is able to partially accommodate the inhibitor and the substrate at the same time.

A Structural Model for the Binding Mode of Eya2 Inhibitors.

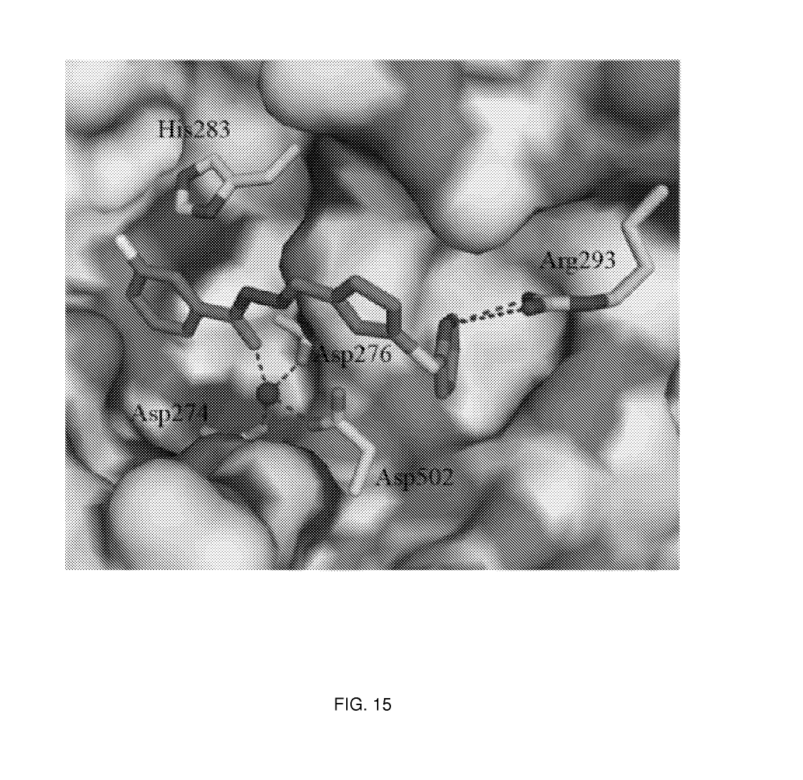

The crystal structure of ED of human Eya2 has been determined (Jung et al., 2010). The inventors docked this series of compounds to the active site of ED using AutoDock 4 (Morris et al., 1998) to evaluate their potential binding interactions and specificities with Eya2. Taking MLS000544460 as an example (FIG. 15), the model revealed that the small molecule inhibitor was accommodated well in the active site, with the N-arylidenebenzohydrazide oxygen atom preferably bound to Mg²⁺ in the catalytic site (FIG. 15). While the metal-chelating geometry was well maintained, MLS000544460 was orientated within the active site in such a manner that the pyridine ring inserted into a hydrophobic pocket forming hydrogen-bonds with Arg 293, whereas the fluorobenzyl group on the other side pointed to the region near residue His283 forming pi-stacking interaction.

### Discussion

Numerous phosphatases have been described as appealing therapeutic targets for drug discovery toward a number of disorders Inhibition of Eya2 phosphatase, for example, might open a new paradigm in the treatment of cancer. However, the difficulty in obtaining small molecules that inhibit phosphatases in a selective manner is well known. This is mostly due to the high degree of similarity of the catalytic domains of this family of enzymes. The Eya phosphatase proteins belong to a unique HAD family of protein Tyr phosphatases, which use an active site Asp instead of a Cys used in most cellular phosphatases. This may provide a unique opportunity to identify small molecules that specifically inhibit the Eya phosphatase.

To this end, the inventors identified a series of first-in-class small molecule inhibitors of Eya2 phosphatase through HTS. The inhibitory potential of available analogs within the chemotype ranged from no activity to low micromolar IC₅₀s, indicating the presence of SAR within the series. These compounds were also confirmed in a malachite green based orthogonal phosphatase assay and do not significantly inhibit other phosphatases including PPM1A, Scp1, and PTP1B. In addition, the inventors investigated the mechanism of action of this series by thermal shift studies. In general the melting temperature (Tₘ) of a protein can be generally used as an indicator of its stability. Compounds that are able to bind a protein specifically can stabilize its structure and lead to an increase in the protein Tₘ. Conversely, compounds that destabilize the structure of the protein or bind to the protein in a non-specific manner (such as binding to unfolded regions of the protein) produce a negative thermal shift (i.e., reduced Tₘ). the inventor demonstrated that, in the absence of additional Mg²⁺, the melting temperature of Eya2 has a strong dependency on EDTA. Higher EDTA concentrations lower the Tₘ of the protein, suggesting that EDTA is able to chelate and extrude residual Mg²⁺ present in the protein active site. This interpretation is in alignment with previous examples where elimination of crucial metals from the active site of enzymes reduces protein stability (Kanaya et al., 1996). The presence of 5 μM Mg²⁺ minimizes the effect of EDTA, even at a concentration of 5 mM, indicating that the protein has a much greater affinity for Mg²⁺ than EDTA does. The inventors also examined the ability of their compounds to shift the melting temperature of Eya2 phosphatase in three different conditions: in the presence of 5 mM EDTA, in the presence of 5 μM Mg²⁺ and in the absence of any additive. They also show that the best compounds induce a positive Tₘ shift in Eya2 in a dose dependent manner in the presence of EDTA. On the contrary, in the presence of Mg²⁺, this series produce a negative Tₘ shift of Eya2 in a dose dependent manner. Interestingly, there appears to be a significant linear correlation between the thermal stabilizing effect of these compounds (ΔTₘ) in the presence of 5 mM EDTA and their inhibitory activities (IC₅₀) in the primary enzymatic assay (FIG. 10D). An increase in the melting temperature correlates with increased binding, and therefore these data indicate that in the presence of 5 mM EDTA, the most active compounds are able to bind Eya 2 phosphatase in a dose-dependent manner. In addition, the correlation of these thermal shift results with the inhibitory capacity (FIG. 10D) suggests that compounds that are able to bind more efficiently exhibit better enzymatic inhibition.

The inventors then evaluated the ability of these compounds to bind metal ions. UV Absorption spectra analysis indicated that there is no association between the compounds and Mg²⁺ in aqueous buffer. A weak but selective association with Mg²⁺ was observed only when an organic solvent such as acetonitrile was utilized. This association was also observed with ¹H NMR spectroscopy. Moreover this weak capacity to coordinate with the metal did not correlate with the inhibitory activity. Therefore, the inventors conclude that the determining factor in this series' mode of action is their binding interaction with Eya2 phosphatase (FIGS. 10A-D), complexing the magnesium (FIGS. 11A-12) from its active site and potentially shifting the metal from its normal position within the protein (FIGS. 9A-C), affecting protein stability (FIGS. 10A-D), and enzymatic activity (FIG. 13). The ability to complex Mg²⁺ is necessary but not sufficient, as strong inhibition can be seen in compound MLS000544460 (which likely binds the protein better, FIGS. 10A-D) but not MLS000585814 even though both compounds have a weak but similar affinity to Mg²⁺ (FIGS. 11A-F). This suggests that these compounds do not mechanistically inhibit the enzymatic activity via simply chelating the metal ion out of the active site (such as EDTA) or causing protein destabilization. In fact, binding to the Eya2 protein most likely plays a paramount role in inhibition and this explains the selectivity of the series versus other Mg²⁺ dependent phosphatases. It is also important to note that these inhibitors show reversible inhibition although they appear to have a slow rate of dissociation from the enzyme. Kinetic studies reveal that the nature of this inhibition may be mixed mode, although additional studies with physiological substrate might reveal a more competitive binding.

To further understand the structural basis of the inhibition mechanism of this series against its protein target Eya2, the inventors performed molecular docking to investigate the plausible binding interactions of these inhibitors in the active site of ED domain. The model indicated that this series of compounds fit well within the catalytic binding site, characterized by a number of favorable binding interactions. The hydrazide functional group acts as a strong metal-chelating moiety, and together with active-site residues Asp274, Asp276 and Asp502, can potentially bind to Mg²⁺ metal ion with a high binding affinity. The inventors' preliminary Molecular Dynamics simulations of these binding complexes show that the distance and metal binding geometries are maintained stably over a time course of 5-ns simulations (data not shown). The Mg²⁺ binding interaction in the active site pocket likely plays a predominant role in the inhibitory activity. Interestingly, the binding interactions of the pyridine group of inhibitor with Arg293 are more dynamic with dramatic conformational changes during MD simulations (data not shown). This is not surprising because Arg293 together with the connecting loop on protein surface are quite flexible. While it is challenging to model inhibitor-induced conformational changes, especially those associated with protein destabilization, the inventors postulate that the strong Mg²⁺-chelating interaction of the N-arylidenebenzohydrazide core and dynamic behavior of the Arg-containing loop causes a significant conformational change of the protein structure in the active site, especially at the C-terminal region which harbors Mg²⁺-coordinating residue Asp502. As a result, the metal-binding coordination is disrupted and the Mg²⁺ ion migrates out of the active site upon inhibitor binding, leading to protein destabilization and inhibition of enzyme activity. The binding model also provides a plausible structural basis for the preliminary structure-activity-relationship already observed. For example, in the low activity analogue MLS000585814, the introduction of a methyl group at ortho position distorted the metal-chelating geometry, as well as affected the pi-stacking interaction with His283 significantly. This might be a plausible explanation for its low activity.

Recently Park et al. (2011) reported the results of a virtual screening approach identifying inhibitors of Eya2 phosphatase. Several compounds were identified with IC₅₀'s between 4.4 to 66.2 μM. Interestingly, the authors also proposed binding models for this inhibitors involving the simultaneous binding of the compound to the protein and the chelation of the active-site Mg²⁺ ion, indicating that this might be a general strategy for modulating this enzyme.

In summary, the inventors investigated the mechanism of action of the first described specific inhibitory series of the ED of Eya2 phosphatase. These compounds bind and stabilize the ED in the absence of Mg²⁺ but destabilize ED in the presence of Mg²⁺. These compounds have a weak but specific binding affinity to Mg²⁺ but not Na⁺. These compounds bind reversibly to the enzyme with a mixed mode inhibition and likely slow dissociation rates. Molecular modeling analysis suggests that this series of compounds form specific binding interactions with Mg²⁺ in the active site of Eya2, providing a plausible model to explain the inhibitory mechanism and destabilization effects, as well as a guide for further lead optimization.

All of the methods and apparatus disclosed and claimed herein can be made and executed without undue experimentation in light of the present disclosure. While the compositions and methods of this invention have been described in terms of particular embodiments, it will be apparent to those of skill in the art that variations may be applied to the methods and apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,683,195
U.S. Pat. No. 4,683,202
U.S. Pat. No. 4,800,159
U.S. Pat. No. 4,883,750
U.S. Pat. No. 5,143,854
U.S. Pat. No. 5,202,231
U.S. Pat. No. 5,242,974
U.S. Pat. No. 5,279,721
U.S. Pat. No. 5,288,644
U.S. Pat. No. 5,324,633
U.S. Pat. No. 5,384,261
U.S. Pat. No. 5,405,783
U.S. Pat. No. 5,412,087
U.S. Pat. No. 5,424,186
U.S. Pat. No. 5,429,807
U.S. Pat. No. 5,432,049
U.S. Pat. No. 5,436,327
U.S. Pat. No. 5,445,934
U.S. Pat. No. 5,468,613
U.S. Pat. No. 5,470,710
U.S. Pat. No. 5,472,672
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,554,501
U.S. Pat. No. 5,556,752
U.S. Pat. No. 5,561,071
U.S. Pat. No. 5,571,639
U.S. Pat. No. 5,580,726
U.S. Pat. No. 5,580,732
U.S. Pat. No. 5,593,839
U.S. Pat. No. 5,599,672
U.S. Pat. No. 5,599,695
U.S. Pat. No. 5,610,287
U.S. Pat. No. 5,624,711
U.S. Pat. No. 5,631,134
U.S. Pat. No. 5,639,603
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,695,940
U.S. Pat. No. 5,700,637
U.S. Pat. No. 5,739,169
U.S. Pat. No. 5,744,305
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,846,945
U.S. Pat. No. 5,847,219
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,871,928
U.S. Pat. No. 5,876,932
U.S. Pat. No. 5,882,864
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 6,004,755
U.S. Pat. No. 6,087,102
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Patent Publn. 2008/0009439
U.S. Patent Pubn. 2009/0163545
Abbondanzo et al., Breast Cancer Res. Treat., 16:182(151), 1990.
Abdelhak et al., Hum. Mol. Genet., 6:2247-2255, 1997.
Abdelhak et al., Nat. Genet., 15:157-164, 1997.
Allred et al., Arch. Surg., 125(1):107-113, 1990.
Ando et al., Febs J., 272(12):3026-3041, 2005.
Arap et al., Cancer Res., 55(6):1351-1354, 1995.
Austin-Ward and Villaseca, Revista Medica de Chile, 126(7):838-845, 1998.
Bakhshi et al., Cell, 41(3):899-906, 1985.
Behbakht et al., Cancer Res., 67:3036-3042, 2007.
Bellus, J. Macromol. Sci. Pure Appl. Chem., A31(1): 1355-1376, 1994.
Bernier et al., Nat. Rev. Cancer, 4:737-747, 2004.
Borsani et al., Hum. Mol. Genet., 8:11-23, 1999.
Brown et al. Immunol. Ser., 53:69-82, 1990.
Bukowski et al., Clinical Cancer Res., 4(10):2337-2347, 1998.
Caldas et al., Cancer Res., 54:3568-3573, 1994.
Cheng et al., Cancer Res., 54(21):5547-5551, 1994.
Christodoulides et al., Microbiology, 144(Pt 11):3027-3037, 1998.
Cleary and Sklar, Proc. Natl. Acad. Sci. USA, 82(21):7439-7443, 1985.
Cleary et al., J. Exp. Med., 164(1):315-320, 1986.
Coletta et al., Cancer Res., 68:2204-2213, 2008.
Coletta et al., Proc. Natl. Acad. Sci. USA, 6478-6483, 2004.
Cook et al., Nature, 458:591-596, 2009.
Davidson et al., J. Immunother., 21(5):389-398, 1998.
De Jager et al., Semin. Nucl. Med., 23(2):165-179, 1993.
Doolittle and Ben-Zeev, Methods Mol, Biol., 109:215-237, 1999.
European Appln. 320 308
European Appln. 329 822
European Appln. EP 373 203
European Appln. EP 785 280
European Appln. EP 799 897
Farabaugh et al., Oncogene, doi: 10.1038/onc.2011.259, 2011 [Epub ahead of print].
Ford et al., Proc. Natl. Acad. Sci. USA, 95:12608-12613, 1998.
Frohman, In: PCR Protocols: A Guide To Methods And Applications, Academic Press, N.Y., 1990.
GB Appln. 2 202 328
Ghosh et al., Mol. Cell., 32:478-490, 2008.
Grifone et al., Development, 132:2235-2249, 2005.
Gulbis and Galand, Hum. Pathol., 24(12):1271-1285, 1993.
Handbook of Pharmaceutical Salts: Properties, and Use, Stahl & Wermuth (Eds.), Verlag Helvetica Chimica Acta, 2002.
Hanibuchi et al., Int. J. Cancer, 78(4):480-485, 1998.
Hellstrand et al., Acta Oncologica, 37(4):347-353, 1998.
Hojo et al., Bull. Chem. Soc. Japan, 75:2629-2636, 2002.
Hui and Hashimoto, Infection Immun., 66(11):5329-5336, 1998.
Hussussian et al., Nat. Genet., 8(1):15-21, 1994.
Ikeda et al., Dev. Biol., 311:53-68, 2007.
Inglese et al., Proc. Natl. Acad. Sci. USA, 103:11473-11478, 2006.
Innis et al., Proc. Natl. Acad. Sci. USA, 85(24):9436-9440, 1988.
Jailkhani et al., Anti-Cancer Agents Med. Chem., 11:64-77, 2011.
Jemc and Rebay, Dev. Biol., 310:416-429, 2007.
Ju et al., Gene Ther., 7(19):1672-1679, 2000.
Jung et al., FASEB J., 24:560-569, 2010.
Kamb et al., Nat. Genet., 8(1):23-26, 1994.
Kamb et al., Science, 2674:436-440, 1994.
Kanaya et al., J. Biol. Chem., 271:32729-32736, 1996.
Kawakami et al., Cancer, 89(2):334-341, 2000.
Kawakami et al., Nucleic Acids Res., 24(2):303-310, 1996.
Kerr et al., Br. J. Cancer, 26(4):239-257, 1972.
Khan et al., Proc. Natl. Acad. Sci. USA, 96(23):13264-13269, 1999.
Krishnan et al., J. Biol. Chem., 284:16066-16070, 2009.
Kwoh et al., Proc. Natl. Acad. Sci. USA, 86:1173, 1989.
Laclef et al., Development, 130:2239-2252, 2003b.
Laclef et al., Mech. Dev., 120:669-679, 2003a.
Li et al., Am. J. Pathol., 160:2181-2190, 2002.
Li et al., Nature, 426:247-254, 2003.
MacBeath and Schreiber, Science, 289(5485):1760-1763, 2000.
Marley et al., Biochem. J., 320(Pt 3):801-806, 1996.
McCoy et al., J. Clin. Invest., 119:2663-2677, 2009.
Micalizzi et al., J. Clin. Invest., 119:2678-2690, 2009.
Mitchell et al., Ann. NY Acad. Sci., 690:153-166, 1993.
Mitchell et al., J. Clin. Oncol., 8(5):856-869, 1990.
Mori et al., Cancer Res., 54(13):3396-3397, 1994.
Morris et al., J. Computat. Chem., 19:1639-1662, 1998.
Morton et al., Arch. Surg., 127:392-399, 1992.
Nakamura et al., In: Handbook of Experimental Immunology (4^{th} Ed.), Weir et al., (eds). 1:27, Blackwell Scientific Publ., Oxford, 1987.
Ng et al., Br. J. Cancer, 95:1050-1055, 2006.
Ng et al., Int. J. Cancer, 127:859-872, 2010.
Nobori et al., Nature (London), 368:753-756, 1995.
Noyes et al., Cell, 133(7):1277-1289, 2008.
Ohara et al., Proc. Natl. Acad. Sci. USA, 86:5673-5677, 1989.
Okamoto et al., Proc. Natl. Acad. Sci. USA, 91(23):11045-11049, 1994.
Oliver et al., Development, 121(3):693-705, 1995.
Orlow et al., Cancer Res, 54(11):2848-2851, 1994.
Orlow et al., Int. J. Oncol., 15(1):17-24, 1994.
Ozaki et al., Development, 131:551-562, 2004.
Pandey and Mann, Nature, 405(6788):837-846, 2000.
Pandey et al., Oncogene, 29:3715-3722, 2010.
Park et al., Chem. Biol. Drug Des., 78(4):642-650, 2011.
PCT Appln. PCT/US2010/043354
PCT Appln. PCT/US87/00880
PCT Appln. PCT/US89/01025
PCT Appln. WO 01/38580
PCT Appln. WO 01/68255
PCT Appln. WO 03/020898
PCT Appln. WO 03/022421
PCT Appln. WO 03/023058
PCT Appln. WO 03/029485
PCT Appln. WO 03/040410
PCT Appln. WO 03/053586
PCT Appln. WO 03/066906
PCT Appln. WO 03/067217
PCT Appln. WO 03/076928
PCT Appln. WO 03/087297
PCT Appln. WO 03/091426
PCT Appln. WO 03/093810
PCT Appln. WO 03/100012
PCT Appln. WO 03/100448A1
PCT Appln. WO 04/020085
PCT Appln. WO 04/027093
PCT Appln. WO 09/923256
PCT Appln. WO 09/936760
PCT Appln. WO 88/10315
PCT Appln. WO 89/06700
PCT Appln. WO 90/07641
PCT Appln. WO 93/17126
PCT Appln. WO 95/11995
PCT Appln. WO 95/21265
PCT Appln. WO 95/21944
PCT Appln. WO 95/35505
PCT Appln. WO 96/31622
PCT Appln. WO 97/10365
PCT Appln. WO 97/27317
PCT Appln. WO 97/43450
PCT Appln. WO 99/35505
Pereira et al., Critical Rev. Toxicol., 41:83-110, 2011.
Pietras et al., Oncogene, 17(17):2235-2249, 1998.
Qin et al., Proc. Natl. Acad. Sci. USA, 95(24):14411-14416, 1998.
Ravindranath and Morton, Intern. Rev. Immunol., 7: 303-329, 1991.
Rayapureddi et al., Nature, 426:295-298, 2003.
Reichenberger et al., Cancer Res., 65(7):2668-2675, 2005.
Rosenberg et al., Ann. Surg. 210(4):474-548, 1989.
Rosenberg et al., N. Engl. J. Med., 319:1676, 1988.
Ruf et al., Proc. Natl. Acad. Sci. USA, 101:8090-8095, 2004.
Sambrook et al., In: Molecular cloning, Cold Spring Harbor Laboratory Press, Cold Spring Harbor, N.Y., 2001.
Serrano et al., Nature, 366:704-707, 1993.
Serrano et al., Science, 267(5195):249-252, 1995.
Southall et al., In: Handbook of Drug Screening, Seethala et al. (Eds.), 2^{nd} Ed.., Informa Healthcare, NY, 442-462, 2009.
Spitz et al., Proc. Natl. Acad. Sci. USA, 95:14220-14225, 1998.
Tierno et al., Nat. Protoc., 2:1134-1144, 2007.
Tonks, Nat. Rev. Mol. Cell. Biol., 7:833-846, 2006.
Tootle et al., Nature, 426:299-302, 2003.
Tsujimoto and Croce, Proc. Natl. Acad. Sci. USA, 83(14):5214-5218, 1986.
Tsujimoto et al., Nature, 315:340-343, 1985.
Tsujimoto et al., Science, 228(4706):1440-1443, 1985. United Kingdom Appln. 8 803 000
van de Vijver et al., N. Engl. J. Med., 347:1999-2009, 2002.
Walker et al., Proc. Natl. Acad. Sci. USA, 89:392-396, 1992.
Wan et al., Int. J. Cancer, 123:32-40, 2008.
Wang et al., Blood, 117:6895-6905, 2011.
Wang et al., Lancet., 365:671-679, 2005.
Xu et al., Development, 130:3085-3094, 2003.
Xu-Dong et al., Clinical Invest. Med., 32:E180-188, 2009.
Yu et al., Cancer Res., 66:1982-1989, 2006.
Yu et al., Nat. Med., 10:175-181, 2004.
Zhang et al., Cancer Res., 65:925-932, 2005.
Zhang et al., J. Assoc. Res. Otolaryngol., 5:295-304, 2004.
Zhang et al., J. Biomol. Screen, 4:67-73, 1999.
Zhang et al., Protein Sci.: Publn. Protein Soc., 19:974-986, 2010.
Zheng et al., Development, 130:3989-4000, 2003.
Zimmerman et al., Genome Res., 7:128-141, 1997.

1. A method of treating cancer in a subject comprising administering to said subject an effective amount of compound that inhibits EYA2 phosphatase activity, said compound having formula: 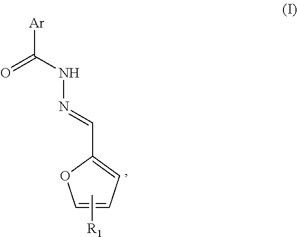
wherein:
Ar is aryl_{(C≦12)}, heteroaryl_{(C≦12)}, or a substituted version of any of these groups;
R₁ is:
hydrogen, hydroxy, amino or mercapto; or
alkyl_{(C≦12)}, aralkyl_{(C≦12)}, heteroaralkyl_{(C≦12)}, acyl_{(C≦12)}, alkoxy_{(C≦12)}, aryloxy_{(C≦12)}, aralkoxy_{(C≦12)}, —CH₂-aralkoxy_{(C≦12)}, hetero-aryloxy_{(C≦12)}, heteroaralkoxy_{(C≦12)}, acyloxy_{(C≦12)}, alkylamino_{(C≦12)}, dialkylamino_{(C≦12)}, arylamino_{(C≦12)}, aralkylamino_{(C≦12)}, heteroarylamino_{(C≦12)}, heteroaralkylamino_{(C≦12)}, amido_{(C≦12)}, alkylthio_{(C≦12)}, arylthio_{(C≦12)}, aralkylthio_{(C≦12)}, heteroarylthio_{(C≦12)}, heteroaralkylthio_{(C≦12)}, acylthio_{(C≦12)}, or a substituted version of any of these groups;
or a pharmaceutically acceptable salt or tautomer thereof.

2. The method of claim 1, wherein Ar is aryl_{(C≦12)} or substituted aryl_{(C≦12)}.

3. The method of claim 2, wherein Ar is aryl_{(c≦8)}.

4. The method of claim 3, wherein Ar is phenyl or methylphenyl.

5. The method of claim 2, wherein Ar is substituted aryl_{(C≦8)}.

6. The method of claim 5, wherein Ar is flurophenyl, chlorophenyl, bromophenyl or nitrophenyl.

7. The method of claim 1, wherein Ar is heteroaryl_{(C≦12)} or substituted heteroaryl_{(C≦12)}.

8. The method of claim 1, wherein said compound is: 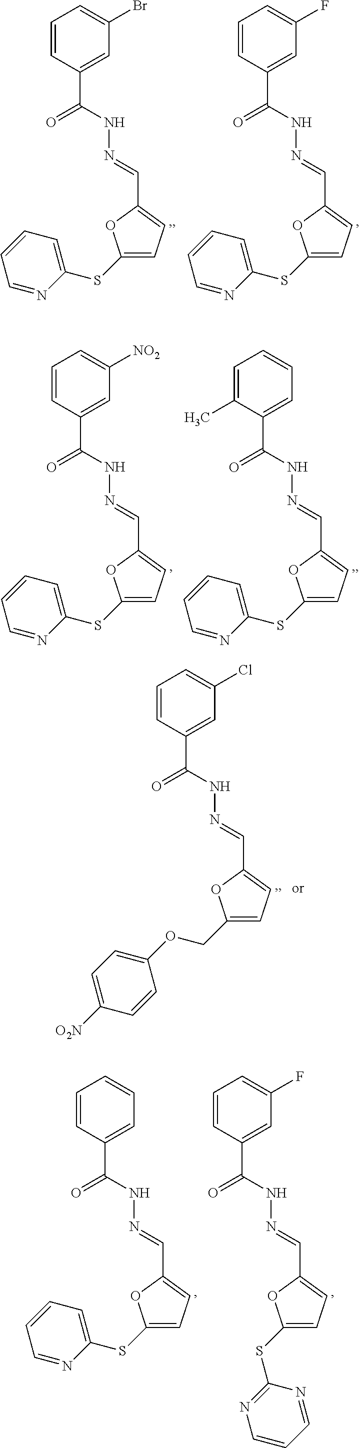 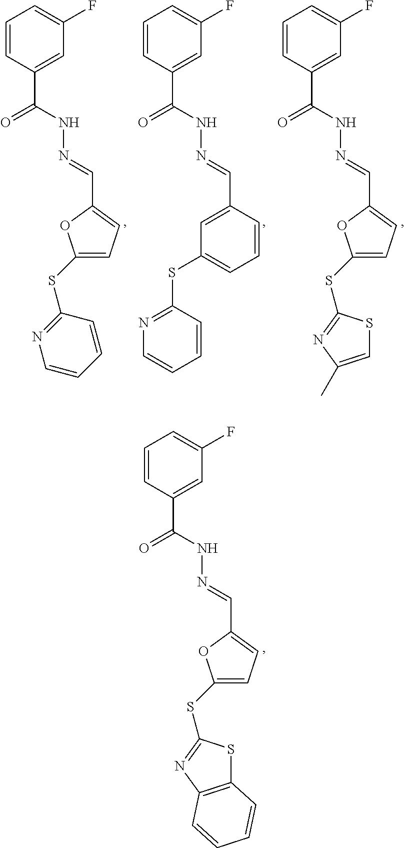
or a pharmaceutically acceptable salt or tautomer thereof.

9. A method of treating cancer in a subject comprising administering to said subject an effective amount of compound that inhibits EYA2 phosphatase activity, said compound having formula: 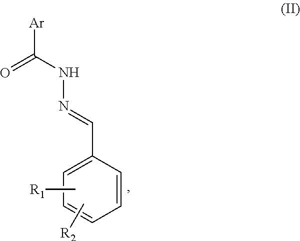
wherein:
Ar is aryl_{(C≦12)}, heteroaryl_{(C≦12)}, or a substituted version of any of these groups;
R₁ and R₂ are each independently:
hydrogen, hydroxy, amino, mercapto, cyano or nitro; or
alkyl_{(C≦12)}, aralkyl_{(C≦12)}, heteroaralkyl_{(C≦12)}, acyl_{(C≦12)}, alkoxy_{(C≦12)}, aryloxy_{(C≦12)}, aralkoxy_{(C≦12)}, —CH₂-aralkoxy_{(C≦12)}, hetero-aryloxy_{(C≦12)}, heteroaralkoxy_{(C≦12)}, acyloxy_{(C≦12)}, alkylamino_{(C≦12)}, dialkylamino_{(C≦12)}, arylamino_{(C≦12)}, aralkylamino_{(C≦12)}, heteroarylamino_{(C≦12)}, heteroaralkylamino_{(C≦12)}, amido_{(C≦12)}, alkylthio_{(C≦12)}, arylthio_{(C≦12)}, aralkylthio_{(C≦12)}, heteroarylthio_{(C≦12)}, heteroaralkylthio_{(C≦12)}, acylthio_{(C≦12)}, or a substituted version of any of these groups;
or a pharmaceutically acceptable salt or tautomer thereof.

10. The method of claim 9, wherein Ar is aryl_{(C≦12)} or substituted aryl_{(C≦12)}.

11. The method of claim 10, wherein Ar is aryl_{(C≦8)}.

12. The method of claim 11, wherein Ar is phenyl.

13. The method of claim 10, wherein Ar is substituted aryl_{(C≦8)}.

14. The method of claim 13, wherein Ar is fluorophenyl.

15. The method of claim 9, wherein Ar is heteroaryl_{(C≦12)} or substituted heteroaryl_{(C≦12)}.

16. The method of claim 15, wherein Ar is substituted heteroaryl_{(C≦8)}.

17. The method of claim 16, wherein Ar is 1-N-methyl,4-chloropyrazol-3-yl.

18. The method of claim 9, wherein said compound is: 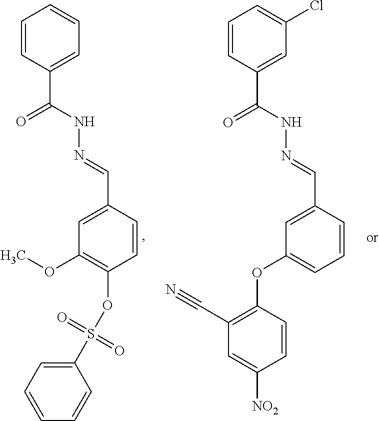 [SMILES: COc1cc(/C=N/NC(=O)c2ccccc2)ccc1OS(=O)(=O)c1ccccc1 ; N#Cc1cc([N+](=O)[O-])ccc1Oc1cccc(/C=N/NC(=O)c2cccc(Cl)c2)c1]
or a pharmaceutically acceptable salt or tautomer thereof.

19. A method of treating cancer in a subject comprising administering to said subject an effective amount of compound that inhibits EYA2 phosphatase activity, said compound selected from the following: 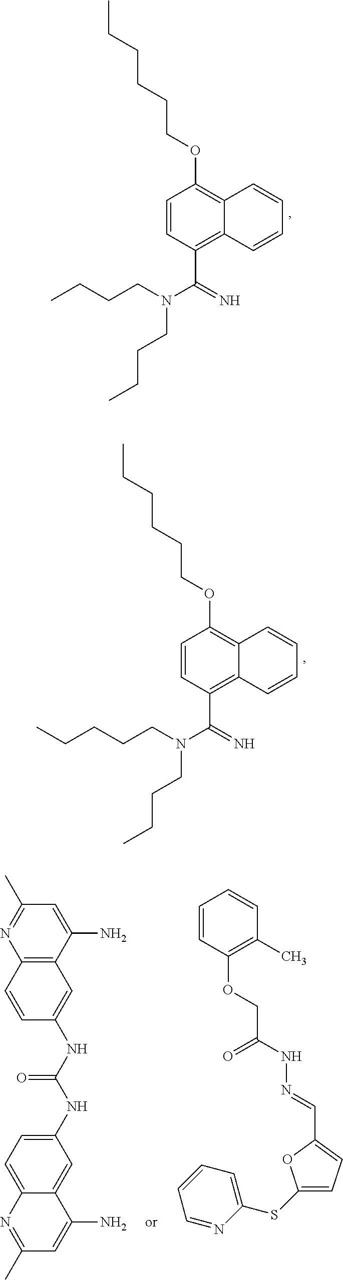
or a pharmaceutically acceptable salt or tautomer of any of the above formulas.

20. A method of treating cancer in a subject comprising administering to said subject an effective amount of compound that inhibits EYA2 phosphatase activity, said compound selected from the following: 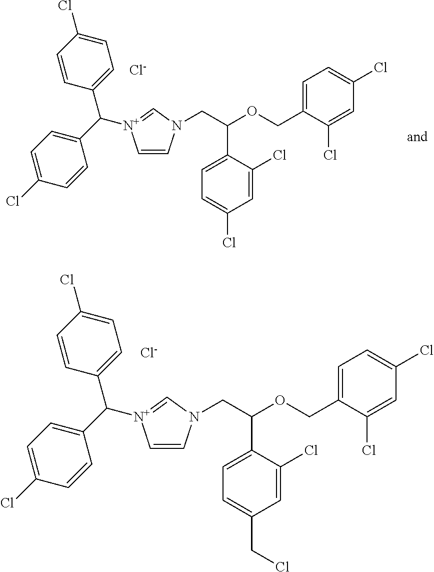 [SMILES: Clc1ccc(C(c2ccc(Cl)cc2)[n+]2ccn(CC(OCc3ccc(Cl)cc3Cl)c3ccc(Cl)cc3Cl)c2)cc1 ; [Cl-] ; ClCc1ccc(C(Cn2cc[n+](C(c3ccc(Cl)cc3)c3ccc(Cl)cc3)c2)OCc2ccc(Cl)cc2Cl)c(Cl)c1 ; [Cl-]]
or a salt thereof having either of the above cations combined with a different pharmaceutically acceptable anion.

21-24. (canceled)

25. The method according to claim 1, wherein the cancer is breast cancer, lung cancer, prostate cancer, ovarian cancer, brain cancer, liver cancer, cervical cancer, colorectal cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia.

26. The method of claim 25, wherein cell of said cancer overexpress EYA2.

27-47. (canceled)
